SEQUENCE LISTING (3 sequences)

SEQ ID NO: 1 - protein, 351 aa, synthetic construct
LQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 2 - protein, 342 aa, synthetic construct
QPKTPVPAQRERAPVSGTQEKNKIRPRGQRDSSDDWEIEASEVMLSTRIGSGSFGTVYKGKWHGDVAVKILKVVDPTPEQFQAFRNEVAVLRKTRHVNILLFMGYMTKDNLAIVTQWCEGSSLYKHLHVQETKFQMFQLIDIARQTAQGMDYLHAKNIIHRDMKSNNIFLHEGLTVKIGDFGLATVKSRWSGSQQVEQPTGSVLWMAPEVIRMQDNNPFSFQSDVYSYGIVLYELMTGELPYSHINNRDQIIFMVGRGYASPDLSKLYKNCPKAMKRLVADCVKKVKEERPLFPQILSSIELLQHSLPKINRSASEPSLHRAAHTEDINACTLTTSPRLPVF

SEQ ID NO: 3 - protein, 10 aa, synthetic construct
  MOD_RES 1: note = pyro-Glutamic acid
  MOD_RES 6: note = O-t-butyl-D-Serine
  MOD_RES 10: note = Amidated azaglycine
EHWSYSLRPG

(19) United States
(12) Patent Application Publication
(10) Pub. No.: US 2026/0176260 A1
(43) Pub. Date: Jun. 25, 2026

(54) DUAL RAF AND TUBULIN INHIBITORS AND METHODS OF USE THEREOF

(71) Applicant: Deciphera Pharmaceuticals, LLC, Waltham, MA (US)

(72) Inventors: Yu Mi Ahn, Waltham, MA (US); Daniel L. Flynn, Waltham, MA (US); Bertrand Le Bourdonnec, Waltham, MA (US); Bryan D. Smith, Waltham, MA (US); Stacie Bulfer, Waltham, MA (US); Jeffery Zwicker, Waltham, MA (US); Salim Javed, Waltham, MA (US); Chase Crawley, Waltham, MA (US)

(21) Appl. No.: 19/416,978

(22) Filed: Dec. 11, 2025

Related U.S. Application Data
(63) Continuation of application No. PCT/US2024/033528, filed on Jun. 12, 2024.
(60) Provisional application No. 63/507,787, filed on Jun. 13, 2023.
(60) Provisional application No. 63/587,821, filed on Oct. 4, 2023.

(51) Int. Cl. C07D 409/04; C07D 417/12; C07D 471/04; C07D 487/04

(52) U.S. Cl. CPC C07D 409/04; C07D 417/12; C07D 471/04; C07D 487/04

(57) ABSTRACT

Described herein are compounds that are dual RAF and tubulin inhibitors and their use in the treatment of disorders such as cancers.

### CROSS REFERENCE

This application is a continuation of International Application No. PCT/US2024/033528 filed Jun. 12, 2024, which claims priority to U.S. Provisional Application No. 63/507,787 filed Jun. 13, 2023 and U.S. Provisional Application No. 63/587,821 filed Oct. 4, 2023, each of which is incorporated herein by reference in their entireties.

### REFERENCE TO A SEQUENCE LISTING XML

This application contains a Sequence Listing which has been submitted electronically in XML format. The Sequence Listing XML is incorporated herein by reference. Said XML file, created on Jun. 5, 2024, is named DCP-118WO_SL.xml and is 5,459 bytes in size.

### BACKGROUND

The BRAF V600X (i.e., V600E) mutant form of BRAF is known to be oncogenic, and there are multiple BRAF inhibitors now marketed to inhibit the signaling of oncogenic BRAF V600E in melanoma and other cancers. BRAF V600E signals as a monomer and is constitutively active independent of upstream control by RAS. The marketed BRAF V600E inhibitors include vemurafenib, dabrafenib, and encorafenib.

Other than BRAF V600X mutants, almost all other oncogenic forms of BRAF signal through the formation of homodimers (BRAF-BRAF dimer) or heterodimers (e.g., BRAF-CRAF dimer) that are refractory to BRAF V600X inhibitors such as vemurafenib, dabrafenib, and encorafenib. Such dimers are formed in cancers driven by BRAF fusions, atypical BRAF mutations, or RAS mutant cancers.

Oncogenic BRAF fusions originate from genomic rearrangements placing the 3-prime portion of the BRAF gene encoding the kinase domain behind another gene at the 5-prime position. The rearrangements result in the expression of oncoproteins that express constitutive kinase activity due to loss of the N-terminal auto-inhibitory domain of BRAF resulting from the genomic rearrangements. These BRAF fusions exhibit constitutive kinase activity due to spontaneous dimerization and as such are capable of aberrant signaling in cancer cells independent of upstream effectors or regulatory mechanisms. Additionally, some 5-prime translocated rearrangement genes contribute the N-terminal domains to be capable of further inducing dimerization, thereby enhancing activating dimerization of the BRAF fusion protein kinase domain. Since the expression of these genomic rearrangements are controlled by the promoter of the 5-prime partner, often there is overexpression of the BRAF fusion transcript due to efficient or excessive promoter activity. BRAF fusions are among the most common kinase translocations in solid tumors. Since their first description in 2005 as oncogenes in papillary thyroid carcinoma, hundreds of tumors in which the BRAF kinase domain is fused to one of more than 110 different 5-prime partner genes have been identified across at least 15 different tumor types. BRAF fusions are found in papillary thyroid carcinoma, astrocytomas, melanomas, and have also been identified in drug resistant EGFR mutant lung cancers. BRAF fusion proteins signal by dimerization in a RAS-independent manner and are resistant to many BRAF inhibitors such as vemurafenib and dabrafenib, that are not capable of inhibiting both protomers of the signaling homodimer BRAF fusions. Rare CRAF fusion proteins have also been demonstrated to be tumor drivers. Such CRAF fusion proteins signal as CRAF-CRAF homodimers.

Other so-called atypical BRAF mutations also lead to spontaneous dimerization and signaling independent of RAS control. Like BRAF fusions, these atypical BRAF mutants signal as aberrant homodimers.

RAS mutant cancers comprise approximately 26-30% of all human cancers. RAS mutant cancers signal through the RAS->RAF->MEK->ERK MAPK signaling pathway. In this signaling cascade, kinase-inactive RAF monomers (comprising ARAF, BRAF, CRAF isoforms) are recruited to oncogenic RAS where RAS induces the formation of kinase-active signaling RAF dimers. A predominant RAF heterodimer that is recruited to mutant RAS is the wildtype BRAF/CRAF heterodimer.

A combinatorial siRNA screening approach identified RAF as a dominant node in RAS mutant cancers, and that codepletion of both BRAF and CRAF, together with depletion of the autophagy gene ATG7, gave the best synthetic lethal inhibition of RAS mutant signaling, and additionally afforded the best therapeutic window for inhibiting signaling in RAS mutant cells versus normal, RAS wildtype cells. Additionally, it has been reported that inhibition of the RAF->MEK->ERK pathway in combination with autophagy-inhibiting agents effectively blocked RAS mutant cancer growth in vitro and in vivo.

Vertical inhibition of the RAF->MEK->ERK pathway through pan inhibition of RAF (specifically BRAF+CRAF) and ERK kinase activities were shown to illicit high synergy in blocking MAPK pathway signaling in KRAS-mutant pancreatic cancer cells, organoid studies, as well as in murine models of KRAS mutant pancreatic cancer. Vertical inhibition of RAF (BRAF+CRAF) and MEK kinase activities was also shown to be synergistic in KRAS mutant tumors.

The importance of inhibiting both BRAF and CRAF isoforms, as well as the requirement of inhibitor successfully binding to and inhibiting both protomers of signaling RAF dimers, has been well demonstrated. Failure of a RAF inhibitor, especially BRAF V600X inhibitors, to successfully bind to and inhibit both protomers of signaling RAF dimers in RAS mutant cancers leads to paradoxical pathway stimulation rather than the desired pathway inhibition. Such BRAF V600X inhibitors are contraindicated for the treatment of RAS mutant cancers.

There is a need to identify RAF inhibitors that can inhibit multiple RAF isoforms. Particularly, there is a need to identify RAF inhibitors that can inhibit both BRAF and CRAF isoforms. Especially, there is a need to identify RAF inhibitors that can inhibit both RAF protomers present in signaling BRAF/BRAF homodimers and both protomers in BRAF/CRAF heterodimers. Such pan RAF inhibitors find utility in the treatment of BRAF V600X driven cancers, atypical BRAF mutated cancers, BRAF fusion cancers, CRAF fusion cancers, and RAS mutant cancers.

Microtubules (MTs), major components of cytoskeletons in eukaryotic cells, play essential roles in multiple cellular functions including maintenance of cell morphology, signal transmission, organelle trafficking, cell motility, cell division and mitosis. These cytoskeletal filaments consist of a- and p-tubulin heterodimers. Microtubule dynamics (assembly and disassembly) are essential for proper mitotic spindle function and completion of mitosis. This highly regulated process is driven by the hydrolysis of GTP on β-tubulin subunits. Thus, disruption of MT dynamics is useful in anti-cancer therapy. Disruption of MT dynamics has been demonstrated to exhibit anti-cancer activity in mutant RAS and mutant RAF driven tumors.

Microtubule targeting agents (MTAs) have antiangiogenic and vascular-disrupting effects in addition to their other effects on cellular function. By affecting the microtubule network, MTAs inhibit endothelial cell proliferation, migration, and tube formation, and cause significant changes in endothelial cell morphology. MTAs have also been evaluated as potential vascular disrupting agents (VDAs). VDAs are known to primarily block the blood flow in solid tumors, leaving the blood vessels in normal tissues intact.

MTAs are classified in three main classes based on their α- or β-tubulin binding site. MTAs binding to the taxane site include the taxanes and the epothilones. These microtubule stabilizing agents bind to fully formed microtubules and prevent the depolymerization of tubulin subunits. In contrast, the vinca alkaloids interact with the tubulin vinca domain found in tubulin dimers and inhibit their polymerization into microtubules (microtubule-destabilizing agents). Colchicine and colchicine binding site inhibitors (CBSIs) interact at a distinct site on tubulin (at the interface of the α- and β-subunit of the tubulin heterodimers) and define the third class of antimitotic agents. Like the vinca alkaloids, these agents also act as microtubule-destabilizing agents.

Compounds altering microtubule function have proven to be highly active in patients with cancer. The taxanes and vinca alkaloids are currently administered in a large variety of indications including solid tumors and hematological malignancies. There are currently no oral CBSI(s) approved as anti-cancer agents.

The major challenges of MTAs currently used clinically (the taxanes in particular) include systemic toxicity, acquired drug resistance, mode of administration limited to the intravenous route, poor water solubility requiring the use of surfactants for intravenous administration with an associated risk of hypersensitivity reactions, and the recurrence of disease when patients are treated in the advanced setting. The MTAs approved for clinical use suffer from dose limiting neurotoxicity and hematopoietic toxicity.

A common mechanism of multidrug resistance (MDR), namely ATP binding cassette (ABC) transporter protein-mediated drug efflux, limits taxanes' efficacy. P-glycoproteins (P-gp, encoded by the MDR1 gene) are important members of the ABC superfamily. P-gp prevents the intracellular accumulation of many cancer drugs by increasing their efflux out of cancer cells. Overexpression of MDR protein, common to most solid tumors, leads to treatment failure and uncontrolled disease progression. Other resistance mechanisms include the upregulation of drug export pump, multidrug resistance-associated protein 1 (MRP1), breast cancer resistance protein (BCRP), or altered expression of tubulin isotypes, and mutations in the β-tubulin gene.

Drug combinations involving agents with distinct anti-cancer mechanisms is commonly used to enhance tumor response and patient survival, especially in the treatment of advanced cancer patients. There is a close interplay between RAS/MAPK pathway and microtubule-dependent MYC regulation pathways. For example, a large genetic loss-of-function siRNA screen identified TUBB3 (encoding bIII-tubulin isoform) as a top pancreatic cancer vulnerability. Genetic silencing of TUBB3 sensitized KRAS mutant pancreatic ductal adenocarcinoma (PDAC) cells to ERK inhibition, indicating that pharmacologic inhibition of proteins that support MYC expression (e.g., tubulin inhibitors) could be an effective therapeutic strategy to target mutant KRAS-dependent PDAC. TUBB3 is also of clinical relevance as overexpression of this β-tubulin isoform has been linked to poor response to microtubule-targeting drugs such as taxanes. Strong TUBB3 expression was most frequently found in various brain tumors, lung cancer, renal cell carcinoma, malignant melanoma, and PDAC. In addition, expression level of TUBB3 was altered in many cancer cells, where aberrant expression of TUBB3 was associated with enhanced chemoresistance and poor prognosis in NSCLC, ovarian cancer, gastric cancer, breast cancer, and uterine serous carcinoma.

Tubulin disrupting agents have also been demonstrated to upregulate MAPK pathway signaling which limits their effectiveness. A dual targeting agent that inhibits MAPK pathway signaling in addition to targeting tubulin would overcome this MAPK pathway reactivation mechanism of resistance.

Dual targeted agents (single molecule interacting with two distinct biological targets) can also offer advantage over combination therapies, and in particular lower risks of drug-drug interactions, more easily predictable pharmacokinetic (PK) profiles, simplification of dosage regimens and increase in patient compliance.

With the high resistance to current MAPK targeted therapeutic agents, identifying agents that exhibit BRAF inhibition and tubulin disruption has the potential to overcome BRAF inhibitors resistance and significantly benefit patients harboring various solid tumors including ovarian, colorectal, and papillary thyroid cancers. Agents which inhibit BRAF and CRAF (so-called pan RAF inhibitors) which also cause tubulin disruption will also potentially benefit patients harboring mutant RAS cancers, providing deeper and more durable anti-cancer efficacy. The compounds, composition, and methods of use described herein are directed toward this need.

### SUMMARY

Described herein, are compounds that are dual RAF and tubulin inhibitors, and methods of use thereof.

In an embodiment, the present disclosure provides a compound represented by Formula I-A:

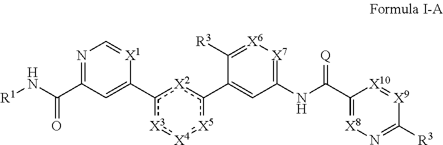

or a pharmaceutically acceptable salt, enantiomer, stereoisomer, or tautomer thereof, wherein:
Q is selected from the group consisting of O and NH;
X¹, X², and X⁵ are each independently selected from the group consisting of CH and N;
X³ and X⁴ are each independently selected from the group consisting of N, CH, C═O, C—O-L-E, C-L-E, C—N(R⁴)-L-E, and N-L-E;
X⁶ is selected from the group consisting of CH and N;
X⁷ is selected from the group consisting of CH, CF, and N;
X⁸ and X¹⁰ are each independently selected from the group consisting of CH, CF, and N;
X⁹ is selected from the group consisting of CR⁵ and N;
provided that not more than two of X², X³, X⁴, and X⁵ is N;
provided that not more than one of X⁶ and X⁷ is N;
provided that not more than one of X⁸, X⁹, and X¹⁰ is N;
provided that when X³ is N, X⁴ is C—O-L-E, C-L-E, C—N(R⁴)-L-E, N, or CH;
provided that when X⁴ is N, X³ is N, CH, C—O-L-E, C-L-E, or C—N(R⁴)-L-E;
provided that when X³ is C═O, X⁴ is N-L-E;
provided that when X⁴ is C═O, X³ is N-L-E;
R¹ is selected from the group consisting of H, alkyl, cycloalkyl, cycloalkylalkyl, alkoxyalkyl, hydroxyalkyl, heterocyclyl, heteroaryl, and haloalkyl,
   wherein the alkyl substituent is independently optionally substituted, at each occurrence, with a substituent selected from the group consisting of amine, halogen, cyano, cycloalkyl, and heterocyclyl;
   wherein the cycloalkyl of cycloalkyl or cycloalkylalkyl substituent is independently optionally substituted, at each occurrence, with a substituent selected from the group consisting of halogen and alkyl;
   wherein the heterocyclyl substituent is independently optionally substituted, at each occurrence, with a substituent selected from the group consisting of halogen, and alkyl;
   wherein the heteroaryl substituent is independently optionally substituted, at each occurrence, with a substituent selected from the group consisting of halogen, and alkyl;
R² is selected from the group consisting of alkyl, H, halogen, and alkoxy;
R³ is selected from the group consisting of H, haloalkyl, alkyl, cycloalkyl, and amine;
R⁴ is selected from the group consisting of H and alkyl;
R⁵ is selected from the group consisting of haloalkyl, cycloalkyl, cyano, H, alkyl, alkoxy, amine, amide, halogen, phosphine oxide, haloalkoxy, and cyanoalkyl, or wherein R⁵ taken together with R³ and the carbon atoms to which they are each attached form a cycloalkyl or a heterocyclyl ring having from 4 to 6 atoms in the ring structure;
L is selected from the group consisting of a direct bond and optionally substituted C₁-C₆alkyl; and
E is selected from the group consisting of H, alkyl, hydroxy, cycloalkyl, haloalkyl, alkoxy, haloalkoxy, alkoxylalkyl, amine, and optionally substituted heterocyclyl wherein the optionally substituted substituent, at each occurrence, is independently selected from the group consisting of alkyl, halogen, amine, hydroxy, oxo, and cyano.

In another embodiment, described herein is a pharmaceutical composition comprising a compound described herein (e.g., a compound of the disclosure as described herein), or a pharmaceutically acceptable salt, enantiomer, stereoisomer, or tautomer thereof, and a pharmaceutically acceptable carrier or excipient.

In another embodiment, described herein is a method of treating a cancer in a patient in need thereof, comprising administering to the patient a therapeutically effective amount of a compound described herein (e.g., a compound of the disclosure as described herein), or a pharmaceutically acceptable salt, enantiomer, stereoisomer, or tautomer thereof, or a composition described herein.

In another embodiment, described herein is a method of treating a disorder selected from the group consisting of histiocytosis, melanoma, multiple myeloma, thyroid cancer, ovarian cancer, colorectal cancer, colon cancer, pancreatic cancer, lung cancer, bladder cancer, gastrointestinal stromal tumors, solid tumors, brain cancers, gliomas, glioblastomas, astrocytomas, blood-borne cancers, hairy cell leukemia, acute myelogenous leukemia (AML), and other cancers caused by activation of the RAS->RAF->MEK->ERK signaling pathway in a patient in need thereof, comprising administering to the patient a therapeutically effective amount of a compound described herein (e.g., a compound of the disclosure as described herein), or a pharmaceutically acceptable salt, enantiomer, stereoisomer, or tautomer thereof, or a composition described herein.

In another embodiments, described herein is a compound described herein (e.g., a compound of the disclosure as described herein), or a pharmaceutically acceptable salt, enantiomer, stereoisomer, or tautomer thereof, or a composition described herein for use in therapy.

In another embodiment, described herein is a compound described herein (e.g., a compound of the disclosure as described herein), or a pharmaceutically acceptable salt, enantiomer, stereoisomer, or tautomer thereof, or a composition described herein for use in a method of treating a cancer in a patient in need thereof.

In another embodiment, described herein is a compound described herein (e.g., a compound of the disclosure as described herein), or a pharmaceutically acceptable salt, enantiomer, stereoisomer, or tautomer thereof, or a composition described herein for use in a method of treating a disorder selected from the group consisting of histiocytosis, melanoma, multiple myeloma, thyroid cancer, ovarian cancer, colorectal cancer, colon cancer, pancreatic cancer, lung cancer, bladder cancer, gastrointestinal stromal tumors, solid tumors, brain cancers, gliomas, glioblastomas, astrocytomas, blood-borne cancers, hairy cell leukemia, acute myelogenous leukemia (AML), and other cancers caused by activation of the RAS->RAF->MEK->ERK signaling pathway in a patient in need thereof.

### BRIEF DESCRIPTION OF THE FIGURES

FIG. 1 is a graph showing the maximum velocity of tubulin polymerization in the presence of increasing concentrations of the known tubulin depolymerizer plinabulin.

FIG. 2 is a graph showing the ratio of pellet (polymerized tubulin) to supernatant (tubulin dimers) compared to the DMSO control for increasing concentrations of the known tubulin depolymerizer plinabulin.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The definitions set forth in this application are intended to clarify terms used throughout this application.

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present disclosure.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents, positions of substituents and/or variables are permissible only if such combinations result in stable compounds.

As used herein, the singular forms “a”, “an”, and “the” encompass plural references unless the context clearly indicates otherwise.

As used herein, the term “herein” means the entire application.

As used herein, “deuterated” mean that at least one hydrogen atom is replaced by deuterium. In any sample of a deuterated compound, some discrete molecules of the compound will likely have hydrogen, rather than deuterium, at the specified position. However, the percent of molecules of the deuterated compound which have deuterium at the specified position will be much greater than would naturally occur. The deuterium at the deuterated position is enriched.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the disclosed compounds can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure result.

As used herein, the term “optionally substituted” refers to the replacement of one to six hydrogen atoms in a given structure with the radical of a specified substituent including, but not limited to: hydroxyl, hydroxyalkyl, alkoxy, halogen, alkyl, aryl, cycloalkyl, heterocyclyl, amino, aminoalkyl, cyano, haloalkyl, haloalkoxy, —OC(═O)—CH₂—Oalkyl. Preferably, “optionally substituted” refers to the replacement of one to four hydrogen atoms in a given structure with the substituents mentioned above. More preferably, one to three hydrogen atoms are replaced by the substituents as mentioned above. It is understood that the substituent can be further substituted.

As used herein, the term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched, and unbranched, carbocyclic and heterocyclic, aromatic and non-aromatic substituents of organic compounds. The permissible substituents can be one or more and the same or different for appropriate organic compounds. For purposes of this application, the heteroatoms such as nitrogen may have hydrogen substituents and/or any permissible substituents of organic compounds described herein which satisfy the valences of the heteroatoms.

Substituents can include any substituents described herein, for example, such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a heteroaralkyl, or an aromatic or heteroaromatic moiety. It will be understood by those skilled in the art that substituents can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN, and the like. Unless specifically stated as “unsubstituted,” references to chemical moieties herein are understood to include substituted variants. For example, reference to an “aryl” group or moiety implicitly includes both substituted and unsubstituted variants.

As used herein, the term “alkyl” refers to a straight chained or branched non-aromatic hydrocarbon which is completely saturated. Typically, a straight chained or branched alkyl group has from 1 to about 20 carbon atoms, preferably from 1 to about 10, e.g., may be C₁-C₁₀alkyl or e.g., C₁-C₆alkyl unless otherwise defined. Examples of straight chained and branched alkyl groups include, but are not limited to, methyl, ethyl, 1-propyl (n-propyl), 2-propyl, n-butyl, sec-butyl, tertbutyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. Moreover, the term “alkyl” used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. The “alkyl” group may be optionally substituted.

The term “Cₓ-C_{y}” when used in conjunction with a chemical moiety, such as, acyl, acyloxy, alkyl, alkenyl, alkynyl, or alkoxy is meant to include groups that contain from x to y carbons in the chain. For example, the term “Cₓ-C_{y}” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. Co alkyl indicates a hydrogen where the group is in a terminal position, a bond if internal.

As used herein, the term “hydrocarbyl” refers to a group that is bonded through a carbon atom that does not have a ═O or ═S substituent, and typically has at least one carbon-hydrogen bond and a primarily carbon backbone but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof. The “hydrocarbyl” group may be optionally substituted.

As used herein, the term “alkoxy” refers to a straight or branched, saturated aliphatic (alkyl) hydrocarbon radical bonded to an oxygen atom that is attached to a core structure. Preferably, alkoxy groups have one to six carbon atoms, i.e., may be C₁-C₆ alkoxy. Examples of alkoxy groups include but are not limited to methoxy, ethoxy, propoxy, isopropoxy, butoxy, isobutoxy, tert-butoxy, pentoxy, 3-methyl butoxy and the like. The “alkoxy” group may be optionally substituted.

As used herein, the term “alkoxyalkyl” refers to an alkyl group (as defined above) substituted with an alkoxy group and may be represented by the general formula alkyl-O-alkyl. Examples of alkoxyalkyl groups include but are not limited to methyl-O-ethylene-, ethyl-O-ethylene-. The “alkoxyalkyl” group may be optionally substituted.

As used herein, the term “haloalkyl” refers to alkyl group (as defined above) is substituted with one or more halogens. A monohaloalkyl radical, for example, may have a chlorine, bromine, iodine, or fluorine atom. Dihalo and polyhaloalkyl radicals may have two or more of the same or different halogen atoms. Examples of haloalkyl include, but are not limited to, chloromethyl, dichloromethyl, trichloromethyl, dichloroethyl, dichloropropyl, fluoromethyl, difluoromethyl, trifluoromethyl, pentafluoroethyl, heptafluoropropyl, difluorochloromethyl, dichlorofluoromethyl, difluoroethyl, difluoropropyl, and the like. The “haloalkyl” group may be optionally substituted.

As used herein, the term “haloalkoxy” refers to radicals wherein one or more of the hydrogen atoms of the alkoxy group are substituted with one or more halogens. Representative examples of “haloalkoxy” groups include, but not limited to, difluoromethoxy (—OCHF₂), trifluoromethoxy (—OCF₃), or trifluoroethoxy (—OCH₂CF₃). The “haloalkoxy” group may be optionally substituted.

As used herein, the term “aryl” includes substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings (fused rings) wherein at least one of the rings is aromatic. e.g., the other cyclic rings can be cycloalkyls, cycloalkenyls, cycloalkynyls, aryls, heteroaryls, and/or heterocyclyls. The term “fused” means that the second ring is attached or formed by having two adjacent atoms in common with the first ring. The term “fused” is equivalent to the term “condensed”. Examples of aryl groups include but are not limited to phenyl, naphthyl, phenanthryl, phenol, aniline, or indanyl and the like. Unless otherwise specified, all aryl groups described herein may be optionally substituted.

As used herein, the terms “polycyclyl”, “polycycle”, and “polycyclic” refer to two or more rings (e.g., cycloalkyls, cycloalkenyls, cycloalkynyls, aryls, heteroaryls, and/or heterocyclyls) in which one or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

As used herein, the term “acyl” refers to a group —C(═O)—R^{w} wherein R^{w} is optionally substituted alkyl. Examples of “acyl” include, but are not limited to, instances where R^{w} is C₁-C₁₀alkyl (C₁-C₁₀acyl) or C₁-C₆alkyl (C₁-C₆acyl). In some embodiments, each occurrence of the optionally substituted substituent is independently selected from the group consisting of H, OH, alkoxy, cyano, F, and amino. Additional examples of “acyl” include —C(═O)—CH₃, —C(═O)—CH₂—CH₃, —C(═O)—CH₂—CH₂—CH₃, or —C(═O)—CH(CH₃)₂.

As used herein, the term “formyl” refers to a group —C(═O)H.

As used herein, the term “sulfonamide” and “sulfonamido” is represented by:

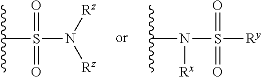

wherein R^{x}, R^{y} and R^{z}, at each occurrence, independently represents a hydrogen, optionally substituted hydrocarbyl group, or R^{z} groups taken together with the N atom to which they are attached complete a heterocycle having from 4 to 8 atoms in the ring structure which may be optionally substituted.

As used herein, the terms “amine” and “amino” refer to both unsubstituted and substituted amines and salts thereof, e.g., a moiety that can be represented by:

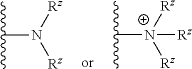

wherein R^{z} independently represent a hydrogen or optionally substituted hydrocarbyl group, or R^{z} groups are taken together with the N atom to which they are attached complete a heterocycle having from 4 to 8 atoms in the ring structure which may be optionally substituted.

As used herein, the terms “amide” and “amido” each refer to a group represented by

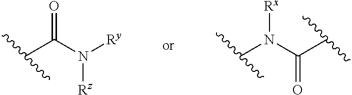

wherein R^{x}, R^{y}, and R^{z} each independently represents a hydrogen or optionally substituted hydrocarbyl group, or R^{y} and R^{z} are taken together with the N atom to which they are attached complete a heterocycle having from 4 to 8 atoms in the ring structure which may be optionally substituted.

As used herein, the term “amidine” refers to a group represented by

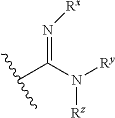

wherein R^{x}, R^{y}, and R^{z} each independently represents a hydrogen or optionally substituted hydrocarbyl group, or R^{y}, and R^{z} groups are taken together with the N atom to which they are attached complete a heterocycle having from 4 to 8 atoms in the ring structure which may be optionally substituted.

As used herein, the term “phosphine oxide” refers to a group represented by

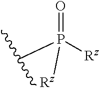

wherein R^{z} each independently represented a represents a hydrogen or optionally substituted hydrocarbyl group.

As used herein, the term “aminoalkyl” refers to an alkyl group substituted with an amino group.

As used herein, the term “amidoalkyl” refers to an alkyl group substituted with an amido group.

As used herein, the term “cyanoalkyl” refers to an alkyl group substituted with a cyano group.

As used herein, the term “alkylthio” refers to a thiol group substituted with an alkyl group and may be represented by the general formula alkyl-S—.

As used herein, the term “thioalkyl” refers to an alkyl group substituted with a thiol group.

As used herein, the term “hydroxyalkyl” refers to an alkyl group substituted with a hydroxy group.

As used herein, the term “cycloalkyl” alone or in combination with other term(s) refers to a cyclic hydrocarbon which is completely saturated. “Cycloalkyl” includes monocyclic, bicyclic, and tricyclic rings. Typically, a monocyclic cycloalkyl group has from 3 to about 10 carbon atoms, more typically 3 to 8 carbon atoms (e.g., C₃-C₁₀cycloalkyl or e.g., C₃-C₆cycloalkyl unless otherwise defined. Examples of monocyclic cycloalkyls include cyclopropyl, cyclobutyl, cyclopentyl, cyclohexyl, cycloheptyl and the like. The second ring of a bicyclic cycloalkyl or, the second or third rings of a tricyclic cycloalkyl, may be selected from saturated, unsaturated and aromatic rings. Cycloalkyl includes bicyclic and tricyclic molecules in which one, two or three or more atoms are shared between the two rings. The term “fused cycloalkyl” refers to a bicyclic or tricyclic cycloalkyl in which each of the rings shares two adjacent atoms with the other ring. The second ring of a fused bicyclic cycloalkyl or, the second or third rings of a fused tricyclic cycloalkyl, may be selected from saturated, unsaturated, and aromatic rings. A “cycloalkenyl” group is a cyclic hydrocarbon containing one or more double bonds. Cycloalkyls can be further substituted with alkyls, alkenyls, alkoxys, alkylthios, aminoalkyls, carbonyl-substituted alkyls, —CF₃, —CN, and the like. A cycloalkyl may alternatively be polycyclic with more than two rings. Examples of polycyclic cycloalkyls include bridged, fused, and spirocyclic carbocyclyls.

As used herein, the term “cycloalkylalkyl” refers to an alkyl group substituted with a cycloalkyl group.

As used herein, the terms “carbocycle,” or “carbocyclic” include bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, 4,5-naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

As used herein, the term “cyano” refers to —CN group.

As used herein, the term “hydroxy” or “hydroxyl” refers to —OH group.

As used herein, the term “halo” or “halogen” alone or in combination with other term(s) means chloro, fluoro, bromo, and iodo.

As used herein, the term “heteroatom” refers an atom of any element other than carbon or hydrogen. Exemplary heteroatoms are nitrogen (N), oxygen (O), sulfur (S), and silicon (Si).

As used herein, the terms “heterocyclyl”, “heterocycloalkyl”, “heterocycle”, and “heterocyclic” refer to a non-aromatic, saturated or partially saturated, including monocyclic, polycyclic (e.g., bicyclic, tricyclic) bridged, or fused, ring system of 3 to 15 member having at least one heteroatom or heterogroup selected from O, N, S, S(O), S(O)₂, NH or C(O) with the remaining ring atoms being independently selected from the group consisting of carbon, oxygen, nitrogen, and sulfur. Examples of “heterocyclyl” include, but are not limited to azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl, indolinyl, indolinylmethyl, 2-azabicyclo[2.2.2]octanyl, azocinyl, chromanyl, xanthenyl and N-oxides thereof. Attachment of a heterocycloalkyl substituent can occur via either a carbon atom or a heteroatom. A heterocycloalkyl group can be optionally substituted with one or more suitable groups by one or more aforesaid groups. Preferably “heterocyclyl” refers to 5- to 6-membered ring selected from the group consisting of azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl and N-oxides thereof. More preferably, “heterocyclyl” includes azetidinyl, pyrrolidinyl, morpholinyl and piperidinyl. All heterocyclyl are optionally substituted by one or more aforesaid groups.

As used herein, the term “heteroaryl” refers to substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also refers to substituted or unsubstituted aromatic or partly aromatic ring systems containing at least one heteroatom and having two or more cyclic rings (bicyclic, tricyclic, or polycyclic), containing 8 to 20 ring atoms, suitably 5 to 10 ring atoms, which may be linked covalently, or fused in which two or more atoms are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. The rings may contain an N or S atom, wherein the N or S atom is optionally oxidized, or the N atom is optionally quaternized. All heteroaryls are optionally substituted. Any suitable ring position of the heteroaryl moiety may be covalently linked to a defined chemical structure. Examples of heteroaryl include, but are not limited to: furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, thiadiazolyl, triazolyl, pyridyl, pyrimidinyl, pyrazinyl, pyridazinyl, triazinyl, benzoxazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, alpha-carboline, indolizinyl, benzoisothiazolyl, benzoxazolyl, pyrrolopyridyl, furopyridinyl, purinyl, benzothiadiazolyl, benzoxadiazolyl, benzotriazolyl, benzotriadiazolyl, 7-azaindolyl, 7-azaindazolyl, pyrrolopyridinyl, pyrrolopyrimidinyl, oxazolonepyridinyl, oxazolonepyrimidinyl, imidazolonepyridinyl, imidazolonepyrimidinyl, pyrazolopyridinyl, pyrazolopyrimidinyl, tetrahydronaphthyridinyl, tetrahydropyridolpyriminyl, dihydronaphthyridinonyl, naphthyridinonyl, oxazinanonepyridinyl, oxazinanonepyrimidinyl, carbazolyl, dibenzothienyl, acridinyl and the like.

As used herein, the terms “sulfone” or “sulfonyl” refer to the group —S(O)₂—R^{6d}wherein R^{6d} represents an optionally substituted hydrocarbyl.

As used herein, [symbol] within a ring, refers to a single or double bond, as valency permits and which results in the formation of a stable ring moiety. In certain embodiments, the ring comprising variables X², X³, X⁴, and X⁵ in compounds of Formula I-A or I-B, comprises single or double bonds, as valency permits, to form a stable aromatic ring moiety.

A “combination therapy” is a treatment that includes the administration of two or more therapeutic agents, e.g., a compound of the disclosure and a MAPK pathway inhibitor, to a patient in need thereof.

“Disease,” “disorder,” and “condition” are used interchangeably herein.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

The MAPK pathway as used herein is the signal transduction pathway comprising RAS->RAF->MEK->ERK.

A “MAPK pathway inhibitor” is an inhibitor of the MAP kinase signaling pathway. Inhibitors of this pathway include RAS inhibitors (e.g., AMG-510, MRTX 849), RAF inhibitors (e.g., dabrafenib, vemurafenib, LY3009120, encorafenib), MEK inhibitors (e.g., trametinib, binimetinib, selumetinib, cobimetinib), and ERK inhibitors (e.g., ulixertinib, SCH772984, LY3214996, ERAS-007). The terms “MAPK pathway inhibitor” and “MAPK kinase inhibitor are used interchangeably herein.

“Pharmaceutically or pharmacologically acceptable” include molecular entities and compositions that do not produce an adverse, allergic, or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds of the disclosure may contain one or more chiral centers and, therefore, exist as stereoisomers. The term “stereoisomers” when used herein consist of all enantiomers or diastereomers. These compounds may be designated by the symbol “R” or “S,” depending on the configuration of substituents around the stereogenic carbon atom, but the skilled artisan will recognize that a structure may denote a chiral center implicitly. These compounds may also be designated by “(+)” and “(−)” based on their optical rotation properties. The presently described compounds encompasses various stereoisomers of these compounds and mixtures thereof. Mixtures of enantiomers or diastereomers may be designated by the symbol “(±)” in nomenclature, but the skilled artisan will recognize that a structure may denote a chiral center implicitly.

In the present specification, the term “therapeutically effective amount” means the amount of the subject compound that will elicit the biological or medical response of a tissue, system or animal, (e.g., mammal or human) that is being sought by the researcher, veterinarian, medical doctor or other clinician. The compounds described herein are administered in therapeutically effective amounts to treat a disorder.

“Treating” includes any effect, e.g., lessening, reducing, modulating, or eliminating, that results in the improvement of the condition, disease, disorder, and the like.

The disclosure also embraces isotopically labeled compounds which are identical to those recited herein, except that one or more atoms are replaced by an atom having an atomic mass or mass number different from the atomic mass or mass number usually found in nature. Examples of isotopes that can be incorporated into the disclosed compounds include isotopes of hydrogen, carbon, nitrogen, oxygen, phosphorus, sulfur, fluorine, and chlorine, such as ²H, ³H, ¹³C, ¹⁴C, ¹⁵N, ¹⁸O, ¹⁷O, ³¹P, ³²p, ³⁵S, ¹⁸F, and ³⁶Cl, respectively. For example, a compound of the disclosure may have one or more H atom replaced with deuterium.

Individual enantiomers and diastereomers of the disclosed compounds can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations and may involve the use of chiral auxiliaries. For examples, see Carreira and Kvaerno, Classics in Stereoselective Synthesis, Wiley-VCH: Weinheim, 2009.

As used herein, “compounds of the disclosure”, comprise compounds of Formula I-A, Formula I-B, Formula I-C, Formula I-D, Formula I-E, Formula I-F, Formula I-G, Formula I-H, or Formula I-J, or a pharmaceutically acceptable salt, enantiomer, stereoisomer, or tautomer thereof.

### Compounds

In an embodiment, provided herein is a compound of Formula I-A:

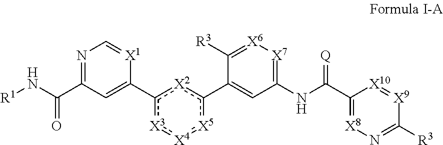

or a pharmaceutically acceptable salt, enantiomer, stereoisomer, or tautomer thereof, wherein:
Q is selected from the group consisting of O and NH;
X¹, X², and X⁵ are each independently selected from the group consisting of CH and N;
X³ and X⁴ are each independently selected from the group consisting of N, CH, C═O, C—O-L-E, C-L-E, C—N(R⁴)-L-E, and N-L-E;
X⁶ is selected from the group consisting of CH and N;
X⁷ is selected from the group consisting of CH, CF, and N;
X⁸ and X¹⁰ are each independently selected from the group consisting of CH, CF, and N;
X⁹ is selected from the group consisting of CR⁵ and N;
provided that not more than two of X², X³, X⁴, and X⁵ is N;
provided that not more than one of X⁶ and X⁷ is N;
provided that not more than one of X⁸, X⁹, and X¹⁰ is N;
provided that when X³ is N, X⁴ is C—O-L-E, C-L-E, C—N(R⁴)-L-E, N, or CH;
provided that when X⁴ is N, X³ is N, CH, C—O-L-E, C-L-E, or C—N(R⁴)-L-E;
provided that when X³ is C═O, X⁴ is N-L-E;
provided that when X⁴ is C═O, X³ is N-L-E;
R¹ is selected from the group consisting of H, alkyl, cycloalkyl, cycloalkylalkyl, alkoxyalkyl, hydroxyalkyl, heterocyclyl, heteroaryl, and haloalkyl, and
   wherein the alkyl substituent is independently optionally substituted, at each occurrence, with a substituent selected from the group consisting of amine, halogen, cyano, cycloalkyl, and heterocyclyl;
   wherein the cycloalkyl of cycloalkyl or cycloalkylalkyl substituent is independently optionally substituted, at each occurrence, with a substituent selected from the group consisting of halogen and alkyl;
   wherein the heterocyclyl substituent is independently optionally substituted, at each occurrence, with a substituent selected from the group consisting of halogen, and alkyl;
   wherein the heteroaryl substituent is independently optionally substituted, at each occurrence, with a substituent selected from the group consisting of halogen, and alkyl;
R² is selected from the group consisting of alkyl, H, halogen, and alkoxy;
R³ is selected from the group consisting of H, haloalkyl, alkyl, cycloalkyl, and amine;
R⁴ is selected from the group consisting of H and alkyl;
R⁵ is selected from the group consisting of haloalkyl, cycloalkyl, cyano, H, alkyl, alkoxy, amine, amide, halogen, phosphine oxide, haloalkoxy, and cyanoalkyl, or wherein Rs taken together with R³ and the carbon atoms to which they are each attached form a cycloalkyl or a heterocyclyl ring having from 4 to 6 atoms in the ring structure;
L is selected from the group consisting of a direct bond and optionally substituted C₁-C₆alkyl; and
E is selected from the group consisting of H, alkyl, hydroxy, cycloalkyl, haloalkyl, alkoxy, haloalkoxy, alkoxylalkyl, amine, and optionally substituted heterocyclyl wherein the optionally substituted substituent, at each occurrence, is independently selected from the group consisting of alkyl, halogen, amine, hydroxy, oxo, and cyano.

In an embodiment, provided herein is a compound of Formula I-B:

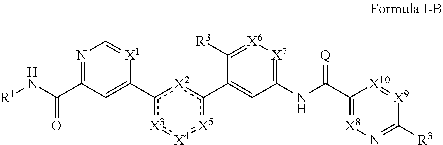

or a pharmaceutically acceptable salt, enantiomer, stereoisomer, or tautomer thereof, wherein:
X¹, X², and X⁵ are each independently selected from the group consisting of CH and N;
X³ and X⁴ are each independently selected from the group consisting of N, CH, C═O, C—O-L-E, C-L-E, C—N(R⁴)-L-E, and N-L-E;
X⁶ is selected from the group consisting of CH and N;
X⁷ is selected from the group consisting of CH, CF and N;
X⁸ and X¹⁰ are each independently selected from the group consisting of CH, CF, and N;
X⁹ is selected from the group consisting of CR⁵ and N;
provided that not more than two of X², X³, X⁴, and X⁵ is N;
provided that not more than one of X⁶ and X⁷ is N;
provided that not more than one of X⁸, X⁹, and X¹⁰ is N;
provided that when X³ is N, X⁴ is C—O-L-E, C-L-E, C—N(R⁴)-L-E, N, or CH;
provided that when X⁴ is N, X³ is N, CH, C—O-L-E, C-L-E, or C—N(R⁴)-L-E;
provided that when X³ is C═O, X⁴ is N-L-E;
provided that when X⁴ is C═O, X³ is N-L-E;
R¹ is selected from the group consisting of H, alkyl, cycloalkyl, alkoxyalkyl, hydroxyalkyl, heterocyclyl, heteroaryl, and haloalkyl,
   wherein the alkyl substituent is independently optionally substituted, at each occurrence, with a substituent selected from the group consisting of amine, halogen, cyano, cycloalkyl, and heterocyclyl;
   wherein the heterocyclyl substituent is independently optionally substituted, at each occurrence, with a substituent selected from the group consisting of halogen, and alkyl;
   wherein the heteroaryl substituent is independently optionally substituted, at each occurrence, with a substituent selected from the group consisting of halogen, and alkyl;
R² is selected from the group consisting of alkyl, H, halogen, and alkoxy;
R³ is selected from the group consisting of H, haloalkyl, alkyl, cycloalkyl, amine;
R⁴ is selected from the group consisting of H and alkyl;
R⁵ is selected from the group consisting of haloalkyl, cycloalkyl, cyano, H, alkyl, alkoxy, amine, amide, halogen, phosphine oxide, haloalkoxy, and cyanoalkyl, or wherein R⁵ taken together with R³ and the carbon atoms to which they are each attached form a cycloalkyl or a heterocyclyl ring having from 4 to 6 atoms in the ring structure;
L is selected from the group consisting of a direct bond and optionally substituted C₁-C₆alkyl; and
E is selected from the group consisting of H, alkyl, hydroxy, cycloalkyl, haloalkyl, alkoxy, haloalkoxy, alkoxylalkyl, amine, and optionally substituted heterocyclyl wherein the optionally substituted substituent, at each occurrence, is independently selected from the group consisting of alkyl, halogen, amine, hydroxy, oxo, and cyano.

In an embodiment, provided herein is a compound of Formula I-C:

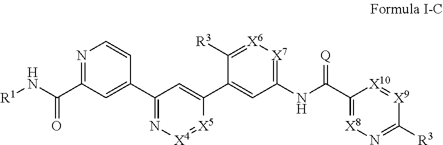

or a pharmaceutically acceptable salt, enantiomer, stereoisomer, or tautomer thereof, wherein:
X⁴is selected from the group consisting of N, CH, C—O-L-E, C-L-E, and C—N(R⁴)-L-E;
X⁵ is selected from the group consisting of CH and N;
X⁶ is selected from the group consisting of CH and N;
X⁷ is selected from the group consisting of CH, CF and N;
X⁸ and X¹⁰ are each independently selected from the group consisting of CH, CF, and N;
X⁹ is selected from the group consisting of CR⁵ and N;
provided that not more than one of X⁴ and X⁵ is N;
provided that not more than one of X⁶ and X⁷ is N;
provided that not more than one of X⁸, X⁹, and X¹⁰ is N;
R¹ is selected from the group consisting of H, alkyl, cycloalkyl, alkoxyalkyl, hydroxyalkyl, heterocyclyl, heteroaryl, and haloalkyl,
   wherein the alkyl substituent is independently optionally substituted, at each occurrence, with a substituent selected from the group consisting of amine, halogen, cyano, cycloalkyl, and heterocyclyl;
   wherein the heterocyclyl substituent is independently optionally substituted, at each occurrence, with a substituent selected from the group consisting of halogen, and alkyl;
   wherein the heteroaryl substituent is independently optionally substituted, at each occurrence, with a substituent selected from the group consisting of halogen, and alkyl;
R² is selected from the group consisting of alkyl, H, halogen, and alkoxy;
R³ is selected from the group consisting of H, haloalkyl, alkyl, cycloalkyl, amine;
R⁴ is selected from the group consisting of H and alkyl;
R⁵ is selected from the group consisting of haloalkyl, cycloalkyl, cyano, H, alkyl, alkoxy, amine, amide, halogen, phosphine oxide, haloalkoxy, and cyanoalkyl, or wherein R⁵ taken together with R³ and the carbon atoms to which they are each attached form a cycloalkyl or a heterocyclyl ring having from 4 to 6 atoms in the ring structure;
L is selected from the group consisting of a direct bond and optionally substituted C₁-C₆alkyl; and
E is selected from the group consisting of H, alkyl, hydroxy, cycloalkyl, haloalkyl, alkoxy, haloalkoxy, alkoxylalkyl, amine, and optionally substituted heterocyclyl wherein the optionally substituted substituent, at each occurrence, is independently selected from the group consisting of alkyl, halogen, amine, hydroxy, oxo, and cyano.

In an embodiment, provided herein is a compound of Formula I-D:

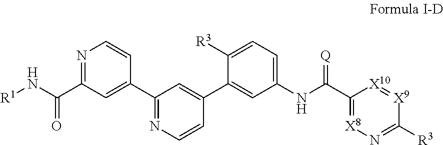

or a pharmaceutically acceptable salt, enantiomer, stereoisomer, or tautomer thereof, wherein:
X⁸ and X¹⁰ are each independently selected from the group consisting of CH, CF, and N;
X⁹ is selected from the group consisting of CR⁵ and N;
provided that not more than one of X⁸, X⁹, and X¹⁰ is N;
R¹ is selected from the group consisting of H, alkyl, cycloalkyl, alkoxyalkyl, hydroxyalkyl, heterocyclyl, heteroaryl, and haloalkyl,
   wherein the alkyl substituent is independently optionally substituted, at each occurrence, with a substituent selected from the group consisting of amine, halogen, cyano, cycloalkyl, and heterocyclyl;
   wherein the heterocyclyl substituent is independently optionally substituted, at each occurrence, with a substituent selected from the group consisting of halogen, and alkyl;
   wherein the heteroaryl substituent is independently optionally substituted, at each occurrence, with a substituent selected from the group consisting of halogen, and alkyl;
R² is selected from the group consisting of alkyl, H, halogen, and alkoxy;
R³ is selected from the group consisting of H, haloalkyl, alkyl, cycloalkyl, amine; and
R⁵ is selected from the group consisting of haloalkyl, cycloalkyl, cyano, H, alkyl, alkoxy, amine, amide, halogen, phosphine oxide, haloalkoxy, and cyanoalkyl, or wherein R⁵ taken together with R³ and the carbon atoms to which they are each attached form a cycloalkyl or a heterocyclyl ring having from 4 to 6 atoms in the ring structure.

In some embodiments, R³ is H.

In some embodiments, R⁵ is haloalkyl, cycloalkyl, or cyano.

In an embodiment, provided herein is a compound of Formula I-E:

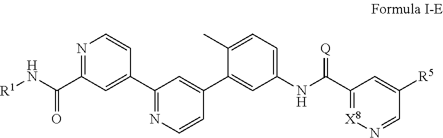

or a pharmaceutically acceptable salt, enantiomer, stereoisomer, or tautomer thereof, wherein:
X⁸ is selected from the group consisting of CH, CF, and N;
R¹ is selected from the group consisting of H, alkyl, cycloalkyl, alkoxyalkyl, hydroxyalkyl, heterocyclyl, heteroaryl, and haloalkyl,
   wherein the alkyl substituent is independently optionally substituted, at each occurrence, with a substituent selected from the group consisting of amine, halogen, cyano, cycloalkyl, and heterocyclyl;
   wherein the heterocyclyl substituent is independently optionally substituted, at each occurrence, with a substituent selected from the group consisting of halogen, and alkyl;
   wherein the heteroaryl substituent is independently optionally substituted, at each occurrence, with a substituent selected from the group consisting of halogen, and alkyl;
   and
R⁵ is selected from the group consisting of haloalkyl, cycloalkyl, cyano, H, alkyl, alkoxy, amine, amide, halogen, phosphine oxide, haloalkoxy, and cyanoalkyl.

In an embodiment, provided herein is a compound of Formula I-F:

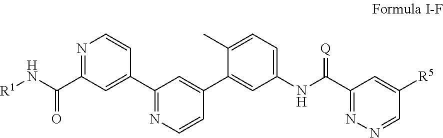

or a pharmaceutically acceptable salt, enantiomer, stereoisomer, or tautomer thereof, wherein:
R¹ is selected from the group consisting of H, alkyl, cycloalkyl, alkoxyalkyl, hydroxyalkyl, heterocyclyl, heteroaryl, and haloalkyl,
   wherein the alkyl substituent is independently optionally substituted, at each occurrence, with a substituent selected from the group consisting of amine, halogen, cyano, cycloalkyl, and heterocyclyl;
   wherein the heterocyclyl substituent is independently optionally substituted, at each occurrence, with a substituent selected from the group consisting of halogen, and alkyl;
   wherein the heteroaryl substituent is independently optionally substituted, at each occurrence, with a substituent selected from the group consisting of halogen, and alkyl;
   and
R⁵ is selected from the group consisting of haloalkyl, cycloalkyl, cyano, H, alkyl, alkoxy, amine, amide, halogen, phosphine oxide, haloalkoxy, and cyanoalkyl.

In some embodiments, R¹ is alkyl, cycloalkyl, or H.

In some embodiments, R⁵ is haloalkyl, cycloalkyl, or cyano.

In an embodiment, provided herein is a compound of Formula I-G:

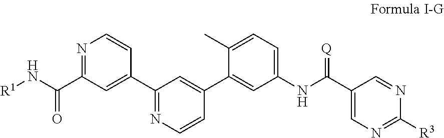

or a pharmaceutically acceptable salt, enantiomer, stereoisomer, or tautomer thereof, wherein:
R¹ is selected from the group consisting of H, alkyl, cycloalkyl, alkoxyalkyl, hydroxyalkyl, heterocyclyl, heteroaryl, and haloalkyl,
   wherein the alkyl substituent is independently optionally substituted, at each occurrence, with a substituent selected from the group consisting of amine, halogen, cyano, cycloalkyl, and heterocyclyl;
   wherein the heterocyclyl substituent is independently optionally substituted, at each occurrence, with a substituent selected from the group consisting of halogen, and alkyl;
   wherein the heteroaryl substituent is independently optionally substituted, at each occurrence, with a substituent selected from the group consisting of halogen, and alkyl;
   and
R³ is selected from the group consisting of H, haloalkyl, alkyl, cycloalkyl, and amine.

In some embodiments, R³ is haloalkyl or cycloalkyl.

In an embodiment, provided herein is a compound of Formula I-H:

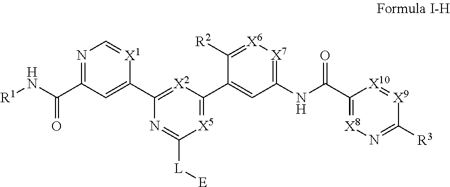

or a pharmaceutically acceptable salt, enantiomer, stereoisomer, or tautomer thereof, wherein:
X¹, X², and X⁵ are each independently selected from the group consisting of CH and N;
X⁶ is selected from the group consisting of CH and N;
X⁷ is selected from the group consisting of CH, CF and N;
X⁸ and X¹⁰ are each independently selected from the group consisting of CH, CF, and N;
X⁹ is selected from the group consisting of CR⁵ and N;
provided that not more than one of X² and X⁵ is N;
provided that not more than one of X⁶ and X⁷ is N;
provided that not more than one of X⁸, X⁹, and X¹⁰ is N;
R¹ is selected from the group consisting of H, alkyl, cycloalkyl, alkoxyalkyl, hydroxyalkyl, heterocyclyl, heteroaryl, and haloalkyl,
   wherein the alkyl substituent is independently optionally substituted, at each occurrence, with a substituent selected from the group consisting of amine, halogen, cyano, cycloalkyl, and heterocyclyl;
   wherein the heterocyclyl substituent is independently optionally substituted, at each occurrence, with a substituent selected from the group consisting of halogen, and alkyl;
   wherein the heteroaryl substituent is independently optionally substituted, at each occurrence, with a substituent selected from the group consisting of halogen, and alkyl;
R² is selected from the group consisting of alkyl, H, halogen, and alkoxy;
R³ is selected from the group consisting of H, haloalkyl, alkyl, cycloalkyl, amine;
R⁴ is selected from the group consisting of H and alkyl;
R⁵ is selected from the group consisting of haloalkyl, cycloalkyl, cyano, H, alkyl, alkoxy, amine, amide, halogen, phosphine oxide, haloalkoxy, and cyanoalkyl, or wherein R⁵ taken together with R³ and the carbon atoms to which they are each attached form a cycloalkyl or a heterocyclyl ring having from 4 to 6 atoms in the ring structure;
L is selected from the group consisting of a direct bond and optionally substituted C₁-C₆alkyl; and
E is selected from the group consisting of H, alkyl, hydroxy, cycloalkyl, haloalkyl, alkoxy, haloalkoxy, alkoxylalkyl, amine, and optionally substituted heterocyclyl wherein the optionally substituted substituent, at each occurrence, is independently selected from the group consisting of alkyl, halogen, amine, hydroxy, oxo, and cyano.

In an embodiment, provided herein is a compound of Formula I-J:

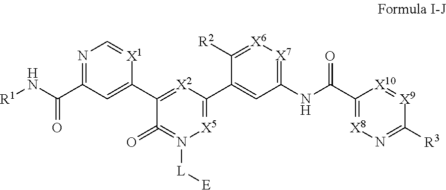

or a pharmaceutically acceptable salt, enantiomer, stereoisomer, or tautomer thereof, wherein:
X¹, X², and X⁵ are each independently selected from the group consisting of CH and N;
X⁶ is selected from the group consisting of CH and N;
X⁷ is selected from the group consisting of CH, CF and N;
X⁸ and X¹⁰ are each independently selected from the group consisting of CH, CF, and N;
X⁹ is selected from the group consisting of CR⁵ and N;
provided that not more than one of X² and X⁵ is N;
provided that not more than one of X⁶ and X⁷ is N;
provided that not more than one of X⁸, X⁹, and X¹⁰ is N;
R¹ is selected from the group consisting of H, alkyl, cycloalkyl, alkoxyalkyl, hydroxyalkyl, heterocyclyl, heteroaryl, and haloalkyl,
   wherein the alkyl substituent is independently optionally substituted, at each occurrence, with a substituent selected from the group consisting of amine, halogen, cyano, cycloalkyl, and heterocyclyl;
   wherein the heterocyclyl substituent is independently optionally substituted, at each occurrence, with a substituent selected from the group consisting of halogen, and alkyl;
   wherein the heteroaryl substituent is independently optionally substituted, at each occurrence, with a substituent selected from the group consisting of halogen, and alkyl;
R² is selected from the group consisting of alkyl, H, halogen, and alkoxy;
R³ is selected from the group consisting of H, haloalkyl, alkyl, cycloalkyl, and amine;
R⁵ is selected from the group consisting of haloalkyl, cycloalkyl, cyano, H, alkyl, alkoxy, amine, amide, halogen, phosphine oxide, haloalkoxy, and cyanoalkyl, or wherein Rs taken together with R³ and the carbon atoms to which they are each attached form a cycloalkyl or a heterocyclyl ring having from 4 to 6 atoms in the ring structure;
L is selected from the group consisting of a direct bond and optionally substituted C₁-C₆alkyl; and
E is selected from the group consisting of H, alkyl, hydroxy, cycloalkyl, haloalkyl, alkoxy, haloalkoxy, alkoxylalkyl, amine, and optionally substituted heterocyclyl wherein the optionally substituted substituent, at each occurrence, is independently selected from the group consisting of alkyl, halogen, amine, hydroxy, oxo, and cyano.

### X¹

In some embodiments, X¹ is CH.

X², X³, X⁴, X⁵

In some embodiments, X² is CH.

In some embodiments, X³ is N.

In some embodiments, X⁴ is selected from the group consisting of N, CH, C—O-L-E, and C—N(R⁴)-L-E. In some embodiments, X⁴ is selected from the group consisting of N and CH. In some embodiments, X⁴ is selected from the group consisting of CO—CH(R⁴)—CH₂—OH and C—NH—CH(R⁴)—CH₂—OH.

In some embodiments, X⁵ is CH.

In some embodiments, the ring containing X², X³, X⁴, and X⁵ is selected from the group consisting of:

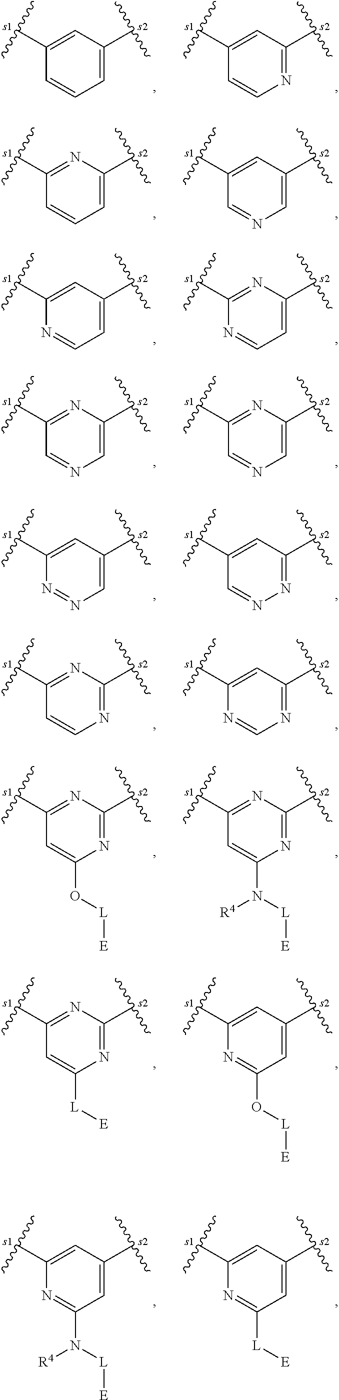

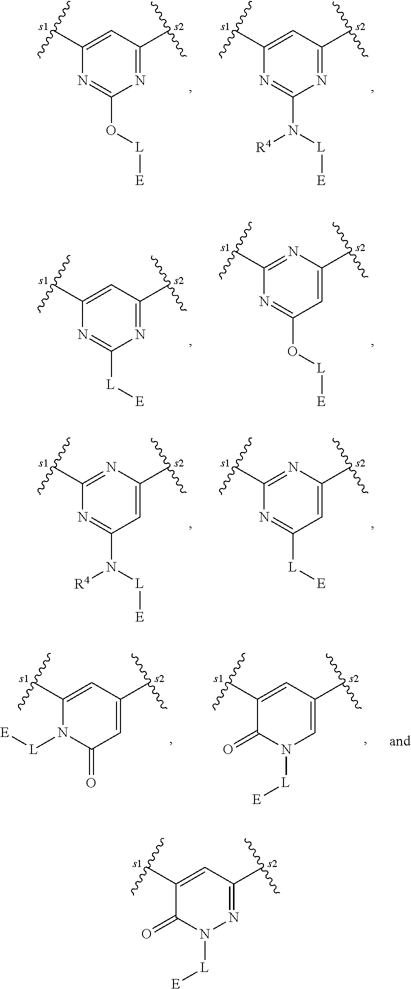

wherein s1 indicates attachment to ring comprising N and substituted with —C(O)NHR¹ and s2 indicates attachment to ring comprising X⁶ and X⁷.
X⁶, X⁷

In some embodiments, X⁶ is CH.

In some embodiments, X⁷ is CH.

X⁸, X⁹, X¹⁰

In some embodiments, X⁸ is N.

In some embodiments, X⁹ is CR⁵.

In some embodiments, R⁵ is selected from the group consisting of alkyl, cycloalkyl, and haloalkyl.

In some embodiments, X¹⁰ is CH.

### R¹

2. In some embodiments, R¹ is selected from the group consisting of H, alkyl, (C3-C8)cycloalkyl, alkoxyalkyl, hydroxyalkyl, heterocyclyl, heteroaryl, and haloalkyl, herein the alkyl substituent is independently optionally substituted, at each occurrence, with a substituent selected from the group consisting of amine, halogen, cyano, (C3-C8)cycloalkyl, and heterocyclyl. In some embodiments, R¹ is selected from the group consisting of H, alkyl, (C3-C8)cycloalkyl, alkoxyalkyl, heterocyclyl, haloalkyl, wherein the alkyl substituent is independently optionally substituted, at each occurrence, with a substituent selected from the group consisting of amine, halogen, and (C3-C8)cycloalkyl. In some embodiments, R¹ is selected from the group consisting of methyl and

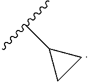 [SMILES: *C1CC1]

### R²

In some embodiments, R² is selected from the group consisting of alkyl and halogen. In some embodiments, R² is selected from the group consisting of methyl and ethyl.

### R³

In some embodiments, R³ is selected from the group consisting of H, alkyl, alkoxy, and haloalkyl.

In some embodiments, R³ is selected from the group consisting of trifluoromethyl and H.

### R⁴

In some embodiments, R⁴ is H.

### R⁵

In some embodiments, R⁵ is selected from the group consisting of H, alkyl, alkoxy, amine, amide, haloalkyl, cycloalkyl, phosphine oxide, halogen, haloalkoxy, cyano, and cyanoalkyl.

In some embodiments, R⁵ is selected from the group consisting of H, trifluoromethyl, isopropyl, cyclopropyl, chloro,

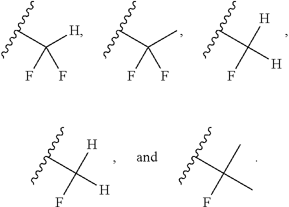

### L

In some embodiments, L is optionally substituted C₁-C₆alkyl.

L is selected from the group consisting of

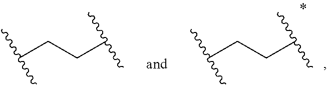

wherein E is attached to the carbon identified with *. In some embodiments, L is selected from the group consisting of

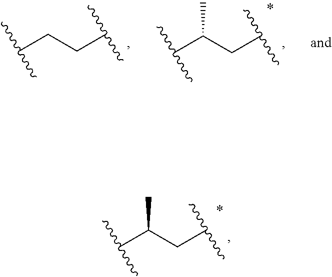

wherein E is attached to the carbon identified with *.

### E

In some embodiments, E selected from the group consisting of H, methyl, and hydroxy.

In some embodiments, the compound is selected from the group consisting of:

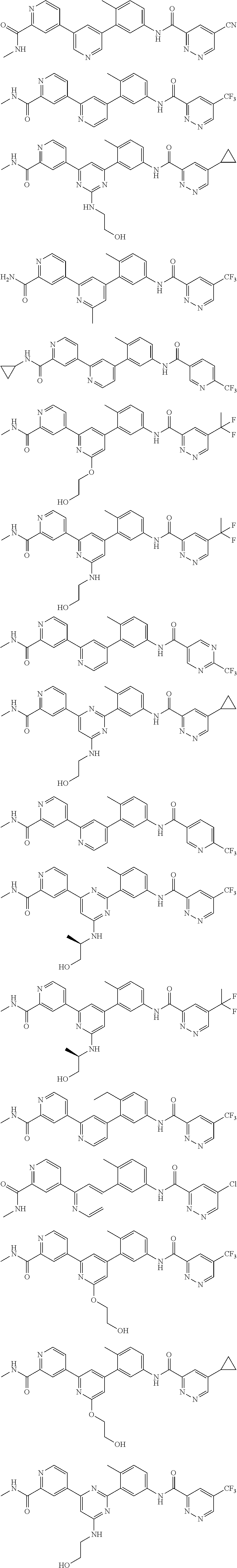

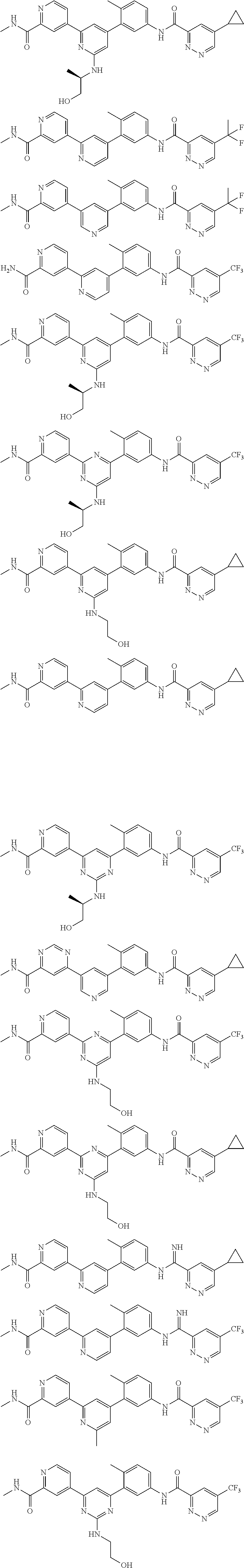

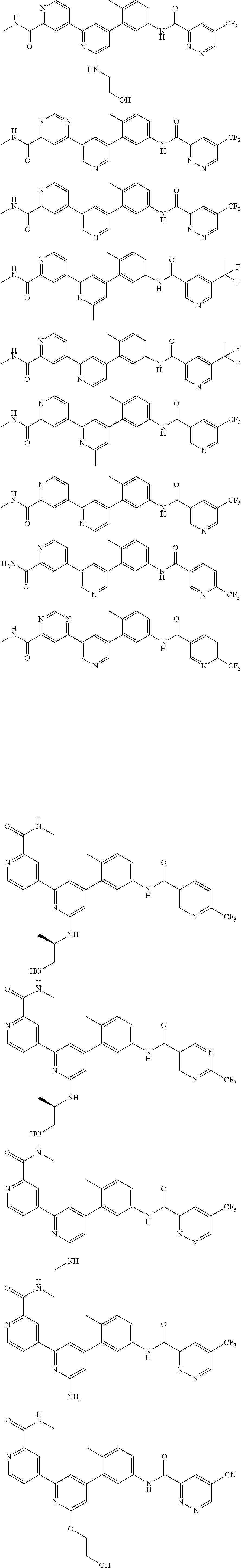

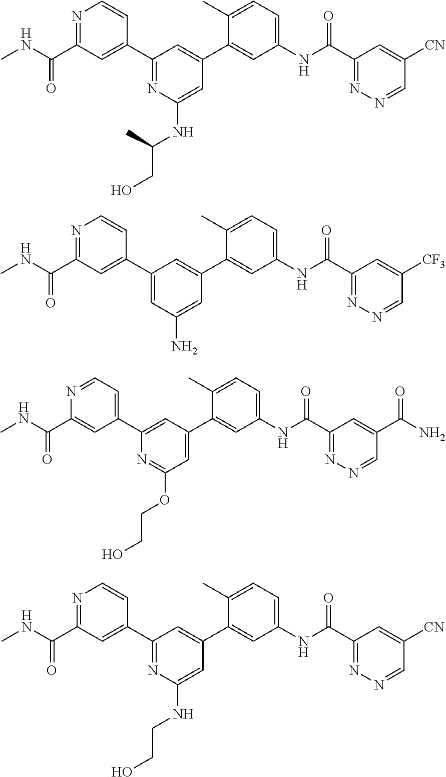

and pharmaceutically acceptable salts, enantiomers, stereoisomers, and tautomers thereof.

### Methods of Treatment

Compounds described herein (e.g., compounds of Formula I-A, Formula I-B, Formula I-C, Formula I-D, Formula I-E, Formula I-F, Formula I-G, Formula I-H, And Formula I-J, or a pharmaceutically acceptable salt, enantiomer, stereoisomer, or tautomer thereof) can act as dual RAF and tubulin inhibitors and are therefore useful in the treatment of diseases and disorders in patients in need thereof, such as cancer. Exemplary cancers include, but are not limited to, melanoma, multiple myeloma, thyroid cancer, ovarian cancer, colorectal cancer, colon cancer, pancreatic cancer, lung cancer, bladder cancer, gastrointestinal stromal tumors, solid tumors, blood-borne cancers, acute myelogenous leukemia (AML), or other cancers caused by activation of the RAS->RAF->MEK->ERK signaling pathway. In some embodiments, a cancer described herein is a BRAF V600X driven cancer, an atypical BRAF mutated cancer, a BRAF fusion cancer, a CRAF fusion cancer, or a RAS mutant cancer. In some embodiments, the cancer has a BRAF oncogenic mutation. In some embodiments, the cancer has a RAS oncogenic mutation. In some embodiments, the RAS oncogenic mutation is RAS Q61R or Q61K mutation. In some embodiments, the cancer has a NF1 oncogenic mutation. In some embodiments, the lung cancer is non-small lung cancer (NSCL). In some embodiments, the colorectal cancer is colon cancer. In some embodiments, the colorectal cancer is rectal cancer.

In some embodiments, provided herein are compounds described herein, or a pharmaceutically acceptable salt, enantiomer, stereoisomer, or tautomer thereof, or a pharmaceutical composition thereof, for use in therapy.

In some embodiments, provided herein are compounds described herein, or a pharmaceutically acceptable salt, enantiomer, stereoisomer, or tautomer thereof, or a pharmaceutical composition thereof, for use in treating a cancer in a patient in need thereof. In some embodiments, the cancer is selected from the group consisting of melanoma, multiple myeloma, thyroid cancer, ovarian cancer, colon cancer, pancreatic cancer, lung cancer, bladder cancer, gastrointestinal stromal tumors, solid tumors, brain cancers, gliomas, glioblastomas, astrocytomas, blood-borne cancers, acute myelogenous leukemia (AML), and other cancers caused by activation of the RAS->RAF->MEK->ERK signaling pathway. In some embodiments, the cancer has a BRAF oncogenic mutation. In some embodiments, the cancer has a RAS oncogenic mutation. In some embodiments, the cancer has a NRAS oncogenic mutation. In some embodiments, the NRAS oncogenic mutation is NRAS Q61R or NRAS Q61K. In some embodiments, the cancer has a KRAS oncogenic mutation. In some embodiments, the KRAS oncogenic mutation is KRAS G12D, KRAS G12V, KRAS G12C, KRAS G12R, or KRAS G13D. In some embodiments, the cancer has a NF1 oncogenic mutation.

In some embodiments, provided herein are compounds described herein, or a pharmaceutically acceptable salt, enantiomer, stereoisomer, or tautomer thereof, or a pharmaceutical composition thereof, for use in treating a disorder selected from the group consisting of melanoma, multiple myeloma, thyroid cancer, ovarian cancer, colon cancer, pancreatic cancer, lung cancer, bladder cancer, gastrointestinal stromal tumors, solid tumors, brain cancers, gliomas, glioblastomas, astrocytomas, blood-borne cancers, acute myelogenous leukemia (AML), and other cancers caused by activation of the RAS->RAF->MEK->ERK signaling pathway in a patient in need thereof.

The compounds provided herein may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, a compound provided herein may be administered orally, subcutaneously, topically, parenterally, by inhalation spray or rectally in dosage unit formulations containing conventional non-toxic pharmaceutically acceptable carriers, adjuvants and vehicles. Parenteral administration may include subcutaneous injections, intravenous or intramuscular injections or infusion techniques.

Treatment can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result is achieved.

### Combination Therapy

Compounds described herein, e.g., a compound of Formula I-A, Formula I-B, Formula I-C, Formula I-D, Formula I-E, Formula I-F, Formula I-G, Formula I-H, And Formula I-J, or a pharmaceutically acceptable salt, enantiomer, stereoisomer, or tautomer thereof, can be administered in combination with one or more additional therapeutic agents to treat a disorder described herein, such as a cancer described herein. For example, provided in the present disclosure is a pharmaceutical composition comprising a compound described herein, e.g., a compound of Formula I-A, Formula I-B, Formula I-C, Formula I-D, Formula I-E, Formula I-F, Formula I-G, Formula I-H, And Formula I-J, or a pharmaceutically acceptable salt, enantiomer, stereoisomer, or tautomer thereof, one or more additional therapeutic agents, and a pharmaceutically acceptable excipient. In some embodiments, a compound of Formula I-A, Formula I-B, Formula I-C, Formula I-D, Formula I-E, Formula I-F, Formula I-G, Formula I-H, And Formula I-J, or a pharmaceutically acceptable salt, enantiomer, stereoisomer, or tautomer thereof, and one additional therapeutic agent is administered. In some embodiments, a compound of Formula I-A, Formula I-B, Formula I-C, Formula I-D, Formula I-E, Formula I-F, Formula I-G, Formula I-H, And Formula I-J, or a pharmaceutically acceptable salt, enantiomer, stereoisomer, or tautomer thereof, and two additional therapeutic agents are administered. In some embodiments, a compound of Formula I-A, Formula I-B, Formula I-C, Formula I-D, Formula I-E, Formula I-F, Formula I-G, Formula I-H, And Formula I-J, or a pharmaceutically acceptable salt, enantiomer, stereoisomer, or tautomer thereof, and three additional therapeutic agents are administered. Combination therapy can be achieved by administering two or more therapeutic agents, each of which is formulated and administered separately. For example, a compound of Formula I-A, Formula I-B, Formula I-C, Formula I-D, Formula I-E, Formula I-F, Formula I-G, Formula I-H, And Formula I-J, or a pharmaceutically acceptable salt, enantiomer, stereoisomer, or tautomer thereof, and an additional therapeutic agent can be formulated and administered separately. Combination therapy can also be achieved by administering two or more therapeutic agents in a single formulation, for example a pharmaceutical composition comprising a compound of Formula I-A, Formula I-B, Formula I-C, Formula I-D, Formula I-E, Formula I-F, Formula I-G, Formula I-H, And Formula I-J, or a pharmaceutically acceptable salt, enantiomer, stereoisomer, or tautomer thereof, as one therapeutic agent and one or more additional therapeutic agents such as a MAPK pathway inhibitor or chemotherapeutic agent. For example, a compound of Formula I-A, Formula I-B, Formula I-C, Formula I-D, Formula I-E, Formula I-F, Formula I-G, Formula I-H, And Formula I-J, or a pharmaceutically acceptable salt, enantiomer, stereoisomer, or tautomer thereof, and an additional therapeutic agent can be administered in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases, even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X-Y-X, X-X-Y, Y-X-Y, Y-Y-X, X-X-Y-Y, etc.

In some embodiments, compounds described herein are combined with other agents including MAPK pathway inhibitors. In some embodiments, the other agent is an inhibitor of RAS. In some embodiments, the other agent is an inhibitor of RAS. In some embodiments, the other agent is an inhibitor of oncogenic KRAS. In some embodiments, the other agent is an inhibitor of KRAS G12C. In some embodiments, the other agent is an inhibitor of RAS. In some embodiments, the other agent is an inhibitor of KRAS G12D. In some embodiments, the other agent is a MEK inhibitor. In some embodiments the other agent is an ERK inhibitor.

In some embodiments, compounds described herein are combined with an immunomodulatory agent. In some embodiments, the immunomodulatory enhances the adaptive immune response. In some embodiments, the immunomodulatory enhances the activity of antigen-presenting cells. In some embodiments, the immunomodulatory agent enhances the anti-tumor activity of myeloid cells including macrophages. In some embodiments, the immunomodulatory enhances the anti-tumor activity of Natural Killer cells. In some embodiments, the immunomodulatory agent enhances the activity of effector T Cells, including cytotoxic T Cells.

In some embodiments, the one or more additional therapeutic agents that may be administered in combination with a compound provided herein can be a MAPK pathway inhibitor. Such MAPK pathway inhibitors include, for example, MEK inhibitors, ERK inhibitors, and Ras inhibitors.

Exemplary MEK inhibitors include, but are not limited to, trametinib, selumetinib, cobimetinib, binimetinib, and pharmaceutically acceptable salts thereof. Exemplary ERK inhibitors include, but are not limited to, include, but are not limited to, ulixertinib, SCH772984, LY3214996, ravoxertinib, VX-Ile, ASN-007, GDC-0994, MK-8353, ASTX-029, LTT462, KO-947, and pharmaceutically acceptable salts thereof. Exemplary Ras inhibitors include, but are not limited to, AMG-510, MRTX849, ARS-1620, ARS-3248, LY3499446, and pharmaceutically acceptable salts thereof.

In some embodiments, the additional therapeutic agents can be immunomodulatory agents including but not limited to anti-PD-1 or anti-PDL-1 therapeutics including pembrolizumab, nivolumab, pidilizumab, cemiplimab, atezolizumab, durvalumab, BMS-936559, or avelumab. In some embodiments, the additional therapeutic agents can be anti-TIM3 (anti-HAVcr2) therapeutics including but not limited to TSR-022 or MBG453, anti-LAG3 therapeutics including but not limited to relatlimab, LAG525, or TSR-033, anti-4-1BB (anti-CD37, anti-TNFRSF9), CD40 agonist therapeutics including but not limited to SGN-40, CP-870,893 or RO7009789, anti-CD47 therapeutics including but not limited to Hu5F9-G4, anti-CD20 therapeutics, anti-CD38 therapeutics, STING agonists including but not limited to ADU-S100, MK-1454, ASA404, or amidobenzimidazoles. In some embodiments, the additional therapeutic agents can be anti-CTLA4 agents including ipilimumab, tremelimumab. In some embodiments, the additional therapeutic agents can be hypomethylating agents including but not limited to azacytidine or decitabine, other immunomodulatory therapeutics including but not limited to epidermal growth factor inhibitors, statins, metformin, angiotensin receptor blockers, thalidomide, lenalidomide, pomalidomide, prednisone, or dexamethasone. In some embodiments, the additional therapeutic agents can be immunotherapeutic agents including targeted therapeutic agents, cancer vaccines, and CAR-T cell therapy.

The compounds described herein may be administered in combination with other therapeutic agents known to treat cancers. Such other therapeutic agents include radiation therapy, anti-tubulin agents, DNA alkylating agents, DNA synthesis-inhibiting agents, DNA intercalating agents, anti-estrogen agents, anti-androgens, steroids, anti-EGFR agents, kinase inhibitors, mTOR inhibitors, PI3 kinase inhibitors, cyclin-dependent kinase inhibitors, CDK4/CDK6 kinase inhibitors, topoisomerase inhibitors, Histone Deacetylase (HDAC) inhibitors, DNA methylation inhibitors, anti-HER2 agents, anti-angiogenic agents, proteasome inhibitors, PARP (poly ADP ribose polymerase) inhibitors, cell cycle regulating kinase inhibitors, thalidomide, lenalidomide, antibody-drug-conjugates (ADCs).

In an embodiment, the additional therapeutic agents can be chemotherapeutic agents including but not limited to an anti-tubulin agents (for example, paclitaxel, paclitaxel protein-bound particles for injectable suspension including but not limited to nab-paclitaxel, eribulin, docetaxel, ixabepilone, vincristine, auristatins, or maytansinoids), vinorelbine, DNA-alkylating agents (including but not limited to cisplatin, carboplatin, oxaliplatin, cyclophosphamide, ifosfamide, temozolomide), DNA intercalating agents or DNA topoisomerase inhibitors (including but not limited to anthracyclines such as doxorubicin, pegylated liposomal doxorubicin, daunorubicin, idarubicin, mitoxantrone, or epirubicin, camptothecins such as topotecan, irinotecan, or exatecan), 5-fluorouracil, capecitabine, cytarabine, decitabine, 5-aza cytadine, gemcitabine and methotrexate.

In some embodiments, the additional therapeutic agents can be kinase inhibitors including but not limited to erlotinib, gefitinib, neratinib, afatinib, osimertinib, lapatanib, crizotinib, brigatinib, ceritinib, alectinib, lorlatinib, everolimus, temsirolimus, abemaciclib, LEE011, palbociclib, cabozantinib, ripretinib, sunitinib, pazopanib, sorafenib, regorafenib, sunitinib, axitinib, dasatinib, imatinib, nilotinib, idelalisib, ibrutinib, BLU-667, Loxo 292, larotrectinib, and quizartinib,

In some embodiments, the additional therapeutic agents can be anti-estrogen agents including but not limited to tamoxifen, fulvestrant, anastrozole, letrozole, and exemestane, anti-androgen agents including but not limited to abiraterone acetate, enzalutamide, nilutamide, bicalutamide, flutamide, cyproterone acetate, steroid agents including but not limited to prednisone and dexamethasone, PARP inhibitors including but not limited to neraparib, olaparib, talazoparib, and rucaparib, topoisomerase I inhibitors including but not limited to irinotecan, camptothecin, exatecan, and topotecan, topoisomerase II inhibitors including but not limited to anthracyclines, etoposide, etoposide phosphate, and mitoxantrone, Histone Deacetylase (HDAC) inhibitors including but not limited to vorinostat, romidepsin, panobinostat, valproic acid, and belinostat, DNA methylation inhibitors including but not limited to DZNep and 5-aza-2′-deoxycytidine, proteasome inhibitors including but not limited to bortezomib and carfilzomib, biological agents including but not limited to trastuzumab, ado-trastuzumab, pertuzumab, cetuximab, and panitumumab.

In some embodiments, the additional therapeutic agents can be anti-angiogenic agents including but not limited to bevacizumab, rebastinib, aflibercept, and AMG386.

In some embodiments, the additional therapeutic agents can be antibody-drug-conjugates (ADCs) including but not limited to ADCs containing DM1, DM4, MMAE, MMAF, or camptothecin payloads, brentuximab vedotin and trastuzumab emtansine, radiotherapy, therapeutic vaccines including but not limited to sipuleucel-T.

In some embodiments, the additional therapeutic agent can be autophagy inhibitors, inhibitors of vesicular trafficking, including but not limited to ULK inhibitors such as ULK1 inhibitors, ULK2 inhibitors, ULK1/ULK2 inhibitors, VPS34 inhibitors, PPT1 inhibitors, or lysosomal blocking agents. In some embodiments, the additional therapeutic agent can be DCC-3116, SAR405, SB02024, hydroxychloroquine, chloroquine, and LYS05.

In some embodiments, the additional therapeutic agent can be EGFR inhibitors including, but not limited to, cetuximab, osimertinib, and afatinib, and pharmaceutically acceptable salts thereof.

In some embodiments, the additional therapeutic agent is selected from a luteinizing hormone-releasing hormone (LHRH) analog, including goserelin and leuprolide.

In some embodiments, the additional therapeutic agent is selected from the group consisting of selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, pemetrexed, erlotinib, dasatanib, nilotinib, decatanib, panitumumab, amrubicin, oregovomab, Lep-etu, nolatrexed, AZD 2171, batabulin, of atumtunab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, alanosine (Sdx 102), talampanel, atrasentan, XR 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)-ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C₁₁, CHIR-258, 3-[5-(methylsulfonylpiperadinemethyl)-indolylj-quinolone, vatalanib, AG-013736, AVE-0005, the acetate salt of [D-Ser(tBu) 6, Azgly 10](pyro-Glu-His-Trp-Ser-Tyr-D-Ser(tBu)-Leu-Arg-Pro-Azgly-NH2 acetate (SEQ ID NO: 3) [C₅₉H8₄N₁₈O₁₄—(C₂H4O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutanide, nilutamide, megestrol acetate, CP-724714; TAK-165, HKI-272, erlotinib, lapatanib, canertinib, ABX-EGF antibody, erbitux, EKB-569, PKI-166, GW-572016, ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, ipilumumab, and mixtures thereof.

### Pharmaceutical Compositions and Kits

Another aspect of this disclosure provides pharmaceutical compositions comprising compounds as disclosed herein formulated together with a pharmaceutically acceptable carrier. In particular, the present disclosure provides pharmaceutical compositions comprising a compound as disclosed herein (e.g., a compound of Formula I-A, Formula I-B, Formula I-C, Formula I-D, Formula I-E, Formula I-F, Formula I-G, Formula I-H, And Formula I-J, or a pharmaceutically acceptable salt, enantiomer, stereoisomer, or tautomer thereof) formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions may be used in the form of a pharmaceutical preparation, for example, in solid, semisolid or liquid form, which contains one or more of the compounds described herein, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound provided herein, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills, and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous, or organic solvents, or mixtures thereof, and powders. Liquid dosage forms for oral administration include pharmaceutically acceptable emulsions, microemulsions, solutions, suspensions, syrups, and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of the present disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In another embodiment, provided are enteral pharmaceutical formulations including a disclosed compound and an enteric material, and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5.

Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives described herein.

Advantageously, provided herein are kits for use by a e.g., a consumer in need of treatment of cancer. Such kits include a suitable dosage form such as those described above and instructions describing the method of using such dosage form to mediate, reduce or prevent inflammation. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . “etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

### EXAMPLES

The compounds described herein can be prepared in a number of ways based on the teachings contained herein and synthetic procedures known in the art. In the description of the synthetic methods described below it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

The following abbreviation are used in this disclosure and have the following definitions: “ADP” is adenosine diphosphate, “Ag₂CO₃” is silver acetate, “aq” is aqueous, “ATP” is adenosine triphosphate, “Ar” is argon gas, “Boc” is t-butylcarbonate, “BSA” is bovine serum albumin, “B2pin2” is 4,4,4′,4′,5,5,5′,5′-octamethyl-2,2′-bi(1,3,2-dioxaborolane), “CaCl₂” is calcium chloride, “cataCXium(R) A-Pd-G2” is Chloro[(di(1-adamanrtyl)-N-butylphosphine)-2-(2-aminobiphenyl)]palladiun(II), “CDCl₃” is chloroform-deuterium, “Cs₂CO₃” is cesium carbonate, “DCM” is dichloromethane, “DIEA” is N,N-diisopropylethylamine, “DMF” is N,N-dimethylformamide, “dppf” is 1,1′-bis(diphenylphosphino)ferrocene, “DMSO-d₆” is dimethylsulfoxide-deuterium, “DSC” is N,N′-Disuccinimidyl carbonate, “DTT” is dithiothreitol, “EDC” is 1-ethyl-3-(3-dimethylaminopropyl)carbodiimide, “ESI” is electrospray ionization, “EtOAc” is ethyl acetate, “EtOH” is ethanol, “GST” is glutathione S-transferase, “h” is hour or hours, “HATU” is hexafluorophosphate azabenzotriazole tetramethyl uronium, “H₂” is hydrogen gas, “HC” is hydrochloric acid, “H₂O” is water, “H₂O₂” is hydrogen peroxide, “IC₅₀” is half maximal inhibitory concentration, “K₂CO₃” is potassium carbonate, “KOAc” is potassium acetate, “LiOH” is lithium hydroxide, “mCPBA” is meta-chloroperoxybenzoic acid, “MeCN” is acetonitrile, “MeOH” is methanol, “mesyl” is methanesulfonyl, “MgSO₄” is magnesium sulfate, “MHz” is megahertz, “min” is minute or minutes, “MS” is mass spectrometry, “m/z” is mass/charge number, “NaCN” is sodium cyanide, “NADH” is nicotinamide adenine dinucleotide, “NaH” is sodium hydride, “NaHCO₃” is sodium bicarbonate, “NaOEt” is sodium methoxide, “Na₂SO₄” is sodium sulfate, “NH₄Cl” is ammonium chloride, “NH₄OH” is ammonium hydroxide, “NMR” is nuclear magnetic resonance, “OMs” is mesylate, “PBS” is phosphate buffered saline, “Pd” is palladium, “Pd/C” is palladium on carbon, “rt” is room temperature which is also known as “ambient temp,” which will be understood to consist of a range of normal laboratory temperatures ranging from 15-25° C., “sat'd.” is saturated, “SFC” is supercritical fluid chromatography, “SM” is starting material, “S_{N}Ar” is nucleophilic aromatic substitution, “T3P” is 1-propanephosphonic acid anhydride, “TBAF” is tetrabutylammonium fluoride, “TBDMS” is tert-butyldimethylsilyl, “TEA” is triethylamine, “TFA” is trifluoroacetic acid, “THF” is tetrahydrofuran, “Tris” is tris(hydroxymethyl)aminomethane, and “XPhos Pd G2” is chloro(2-dicyclohexylphosphino-2′,4′,6′-triisopropyl-1,1′-biphenyl)(2′-amino-1,1′-biphenyl-2-yl)palladium(II).

### General Chemistry

Exemplary compounds described herein are available by the general synthetic methods illustrated in the Schemes below, intermediate preparations, and the accompanying Examples.

### Synthetic Schemes

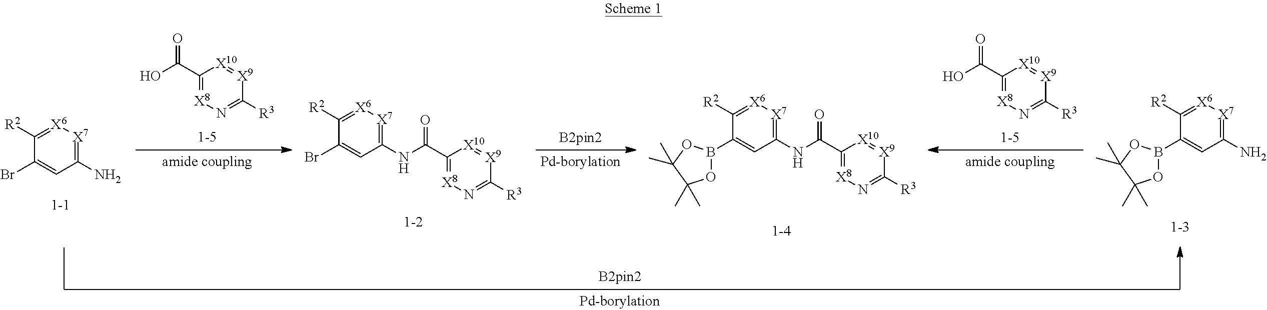

Scheme 1 illustrates an exemplary preparation of boronates 1-4. Amines 1-1 (commercially available or synthesized by those skilled in the art) react with carboxylic acids 1-5 (commercially available or synthesized by those skilled in the art) to afford amides 1-2. Compounds 1-2 upon borylation provide boronates 1-4. Borylation is a well-documented reaction to those skilled in the art (for example, Pd (0) catalyzed reaction with B2pin2). Alternatively, boronates 1-4 can be prepared by amide coupling reaction of 1-3 (commercially available or synthesized by Pd-catalyzed borylation from 1-1) with carboxylic acids 1-5 in the presence of coupling reagents such as HATU, T3P, and EDC.

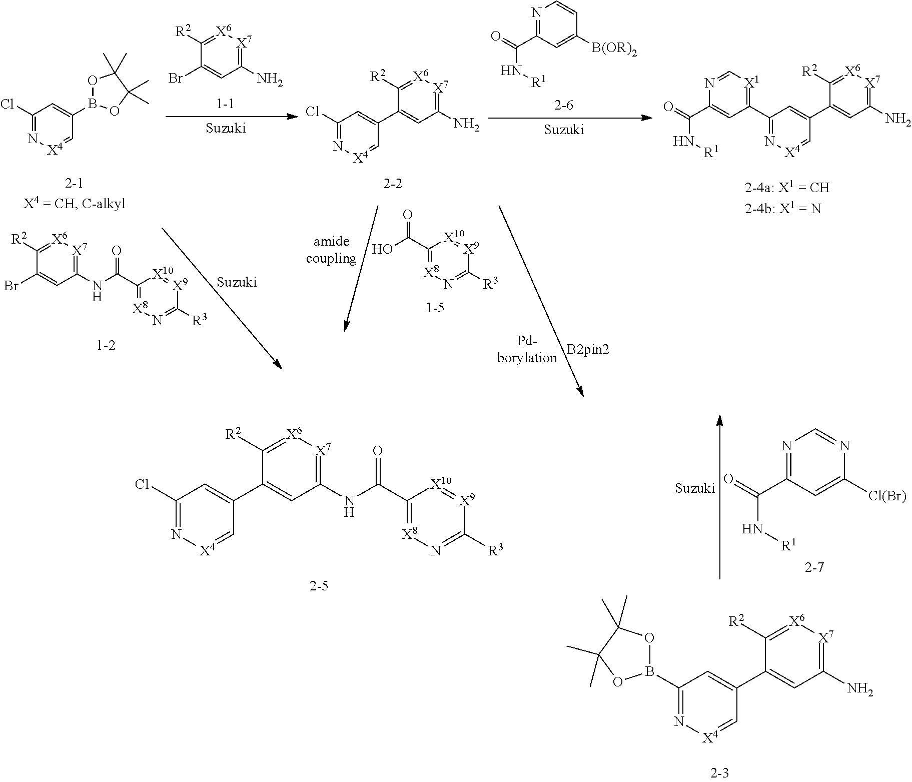

Scheme 2 illustrates an exemplary preparation of intermediates 2-4, and 2-5. Treatment of boronates 2-1 (X⁴=CH, C-alkyl; commercially available or synthesized by those skilled in the art) with bromides 1-1 (commercially available or synthesized by those skilled in the art) in the presence of a palladium catalyst (Suzuki reaction) affords 2-2 (X⁴=CH, C-alkyl). Subsequent Suzuki reaction of 2-2 with boronates or boronic acids 2-6 (commercially available or synthesized by those skilled in the art) afford 2-4a (X¹=CH). Alternatively, borylation of chlorides 2-2 with B2pin2 converts to boronates 2-3 which can be reacted with chlorides (or bromides) 2-7 (commercially available or synthesized by those skilled in the art) under a typical Suzuki reaction condition to afford 2-4b (X¹=N). In another embodiment, amides 2-5 can be prepared by (1) amide coupling reaction of 2-2 with carboxylic acids 1-5 (commercially available or synthesized by those skilled in the art), and (2) Suzuki reaction of 2-1 with bromides 1-2.

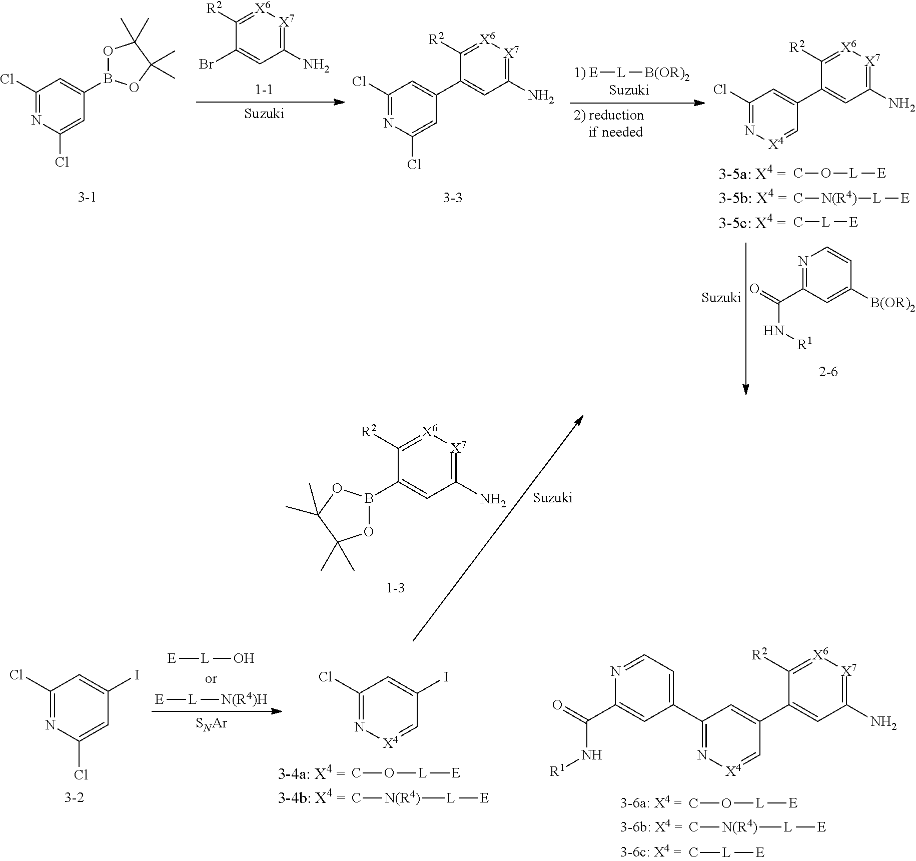

Scheme 3 illustrates an exemplary preparation of intermediates 3-6a, 3-6b, and 3-6c. Treatment of 2,6-dichloro-4-(4,4,5,5-tetramethyl-1,3,2-dioxaborolan-2-yl)pyridine, 3-1 with bromides 1-1 in the presence of a palladium catalyst (Suzuki reaction) affords 3-3. Compounds 3-3 react with boronates or boronic acids E-L-B(OR)₂ to afford compounds 3-5c. If compounds 3-5c (X⁴=C-L-E) contain an unsaturated functionality such as a double bond, or a triple bond, hydrogenation can be proceeded in the presence of Pd catalyst. 2,6-Dichloro-4-iodopyridine, 3-2 reacts with alcohols E-L-OH or amines E-L-N(R⁴)H in aprotic solvent such as NMP, in the presence of base such as K₂CO₃, at elevated temp to afford 3-4a (X⁴=C—O-L-E) and 3-4b (X⁴=C—N(R⁴)-L-E), respectively. Suzuki reaction of each 3-4a and 3-4b with boronates 1-3 under Pd(O) catalyzed coupling conditions affords 3-5a, and 3-5b, respectively. Finally, each 3-5a, 3-5b, and 3-5c reacts with boronates or boronic acids 2-6 under Suzuki reaction conditions to afford 3-6a (X⁴=C—O-L-E), 3-6b (X⁴=C—N(R⁴)-L-E), and 3-6c (X⁴=C-L-E).

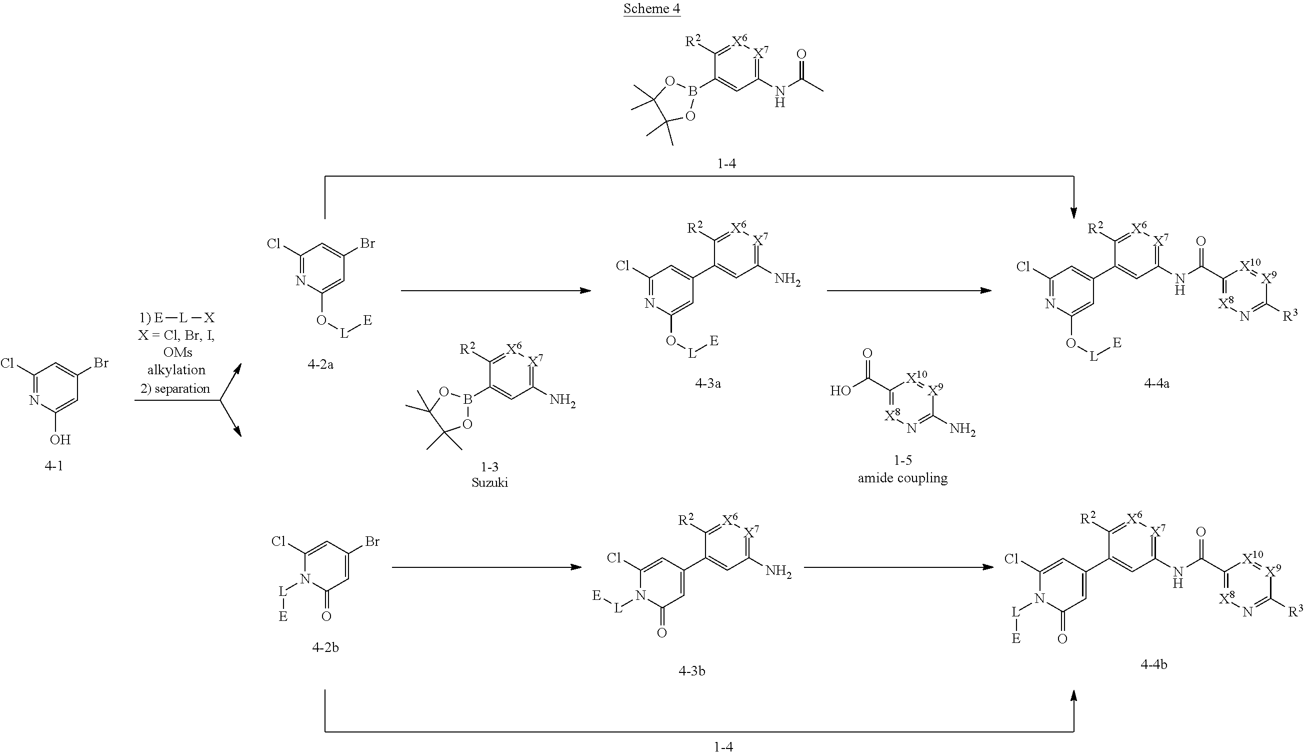

Scheme 4 illustrates an exemplary preparation of intermediates 4-4a and 4-4b. 4-bromo-6-chloropyridin-2-ol (4-1) reacts with alkylating reagents E-L-X (X=Cl, Br, I, OMs) in the presence of base such as Ag₂CO₃ in an aprotic solvent like toluene to produce a mixture of O-alkylated (4-2a) and N-alkylated (4-2b) compounds which can be separated by a suitable method such as SFC purification, crystallization, or chromatography. Each of 4-2a and 4-2b reacts with boronates 1-3 under Suzuki reaction conditions to afford 4-3a and 4-3b, respectively. Amide coupling reaction of 4-3a and 4-3b with carboxylic acids 1-5 affords 4-4a and 4-4b, respectively. In another embodiment, Suzuki reaction of 4-2a and 4-2b with boronates 1-4 affords 4-4a and 4-4b, respectively.

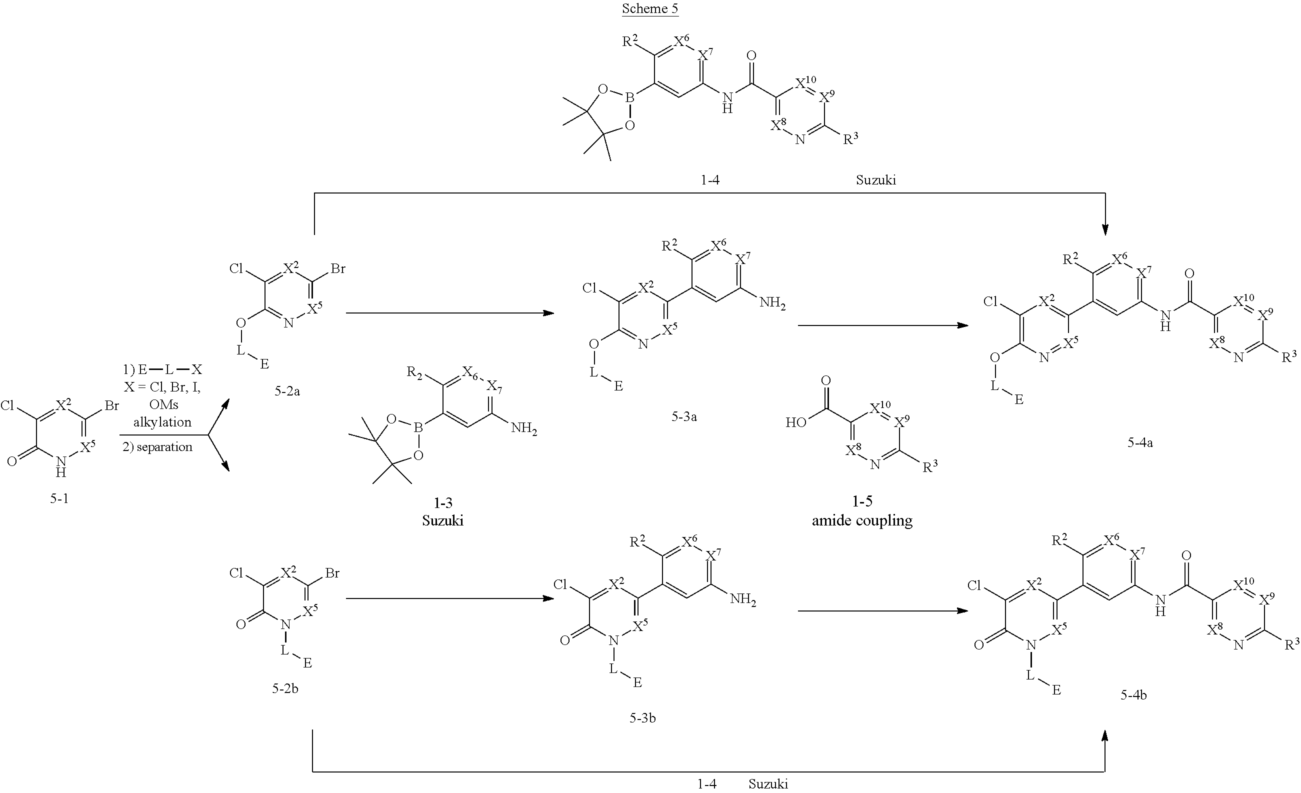

Scheme 5 illustrates an exemplary preparation of intermediates 5-4a and 5-4b. In a similar manner as described in scheme 4, 5-1 reacts with alkylating reagents F3-L³-X (X═Cl, Br, I, OMs) to produce a mixture of O-alkylated (5-2a) and N-alkylated (5-2b) compounds which can be separated by a suitable method well known to those skilled in the art. Suzuki reaction of 5-2a and 5-2b with boronates 1-3 affords 5-3a and 5-3b, respectively. Finally, each of 5-3a and 5-3b upon treatment with carboxylic acids 1-5 under amide coupling conditions affords 5-4a and 5-4b, respectively. In another embodiment, Suzuki reaction of 5-2a and 5-2b with boronates 1-4 affords 5-4a and 5-4b, respectively.

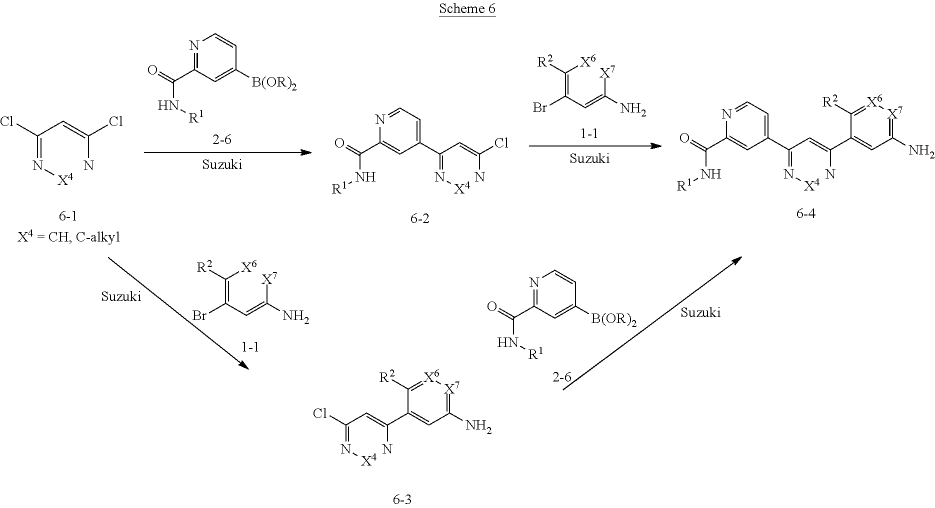

Scheme 6 illustrates an exemplary preparation of intermediates 6-4. Dichlorides 6-1 (X⁴=CH, C-alkyl; commercially available or synthesized by those skilled in the art) reacts with boronates or boronic acids 2-6 in the presence of a palladium catalyst (Suzuki reaction) to afford 6-2. Subsequent palladium catalyzed Suzuki reaction of 6-2 with bromides 1-1 affords intermediates 6-4. In a similar manner, dichlorides 6-1 (commercially available or synthesized by those skilled in the art) react with bromides 1-1 under Suzuki reaction conditions to afford 6-3. Subsequent palladium catalyzed Suzuki reaction of 6-3 with boronates or boronic acids 2-6 affords intermediates 6-4.

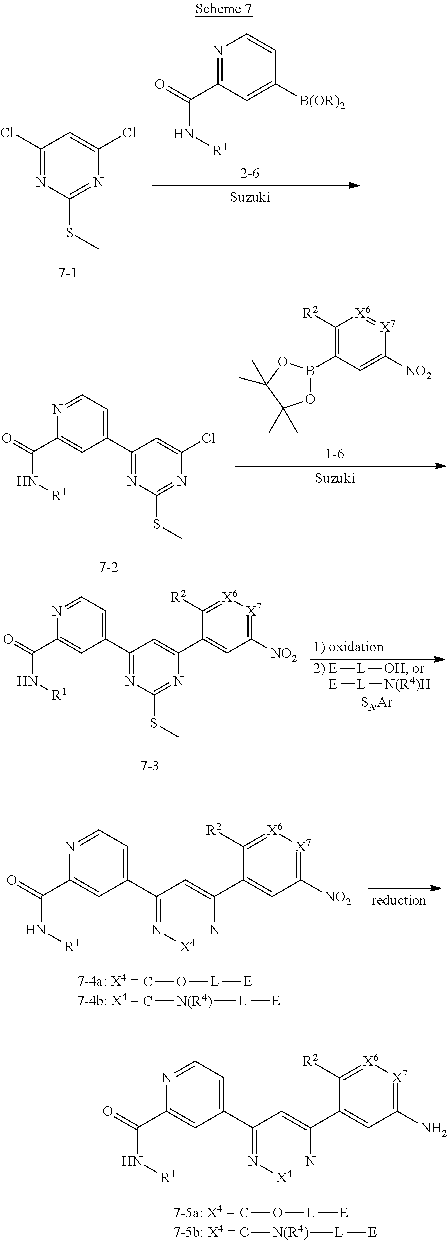

Scheme 7 illustrates an exemplary preparation of intermediates 7-5a (X⁴=C—O-L-E) and 7-5b (X⁴=C—N(R⁴)-L-E). 4,6-Dichloro-2-(methylthio)pyrimidine (7-1) reacts with boronates or boronic acids 2-6 by Suzuki reaction to afford 7-2. Compounds 7-2 react with boronates 1-6 in the presence of a palladium catalyst (Suzuki reaction) to afford 7-3. Compounds 7-3 can be converted to compounds 7-4a (X⁴═C—O-L-E) and 7-4b (X⁴═C—N(R4)-L-E) respectively according to a well-documented reaction sequence: an oxidation reaction using conditions known in the art such as mCPBA in DCM, followed by substitution reaction of the resulting intermediate with commercially available alcohols E-L-OH or amines E-L-N(R⁴)H. Finally, nitro reduction of 7-4a and 7-4b under mild reducing conditions (zinc or iron metal with ammonium chloride) affords the corresponding amines 7-5a (X⁴=C—O-L-E) and 7-5b (X⁴=C—N(R⁴)-L-E) respectively.

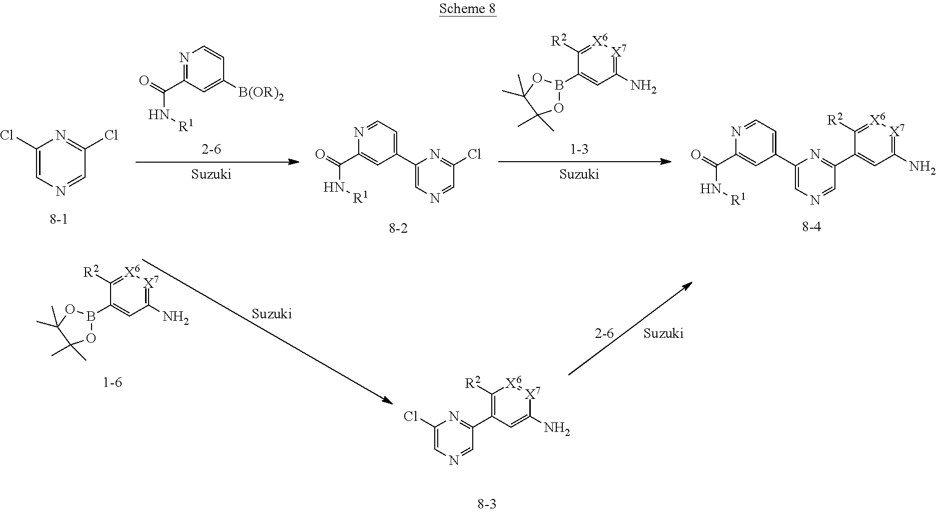

Scheme 8 illustrates an exemplary preparation of intermediates 8-4. 2,6-Dichloropyrazine (8-1) reacts with boronates or boronic acids 2-6 by Suzuki reaction to afford 8-2 which can be subjected to another Suzuki reaction with boronates 1-3 to furnish 8-4. In a similar manner, 2,6-dichloropyrazine 8-1 reacts with boronates 1-3 by Suzuki reaction to afford 8-3 which can be subjected to another Suzuki reaction with boronates 2-6 to furnish 8-4.

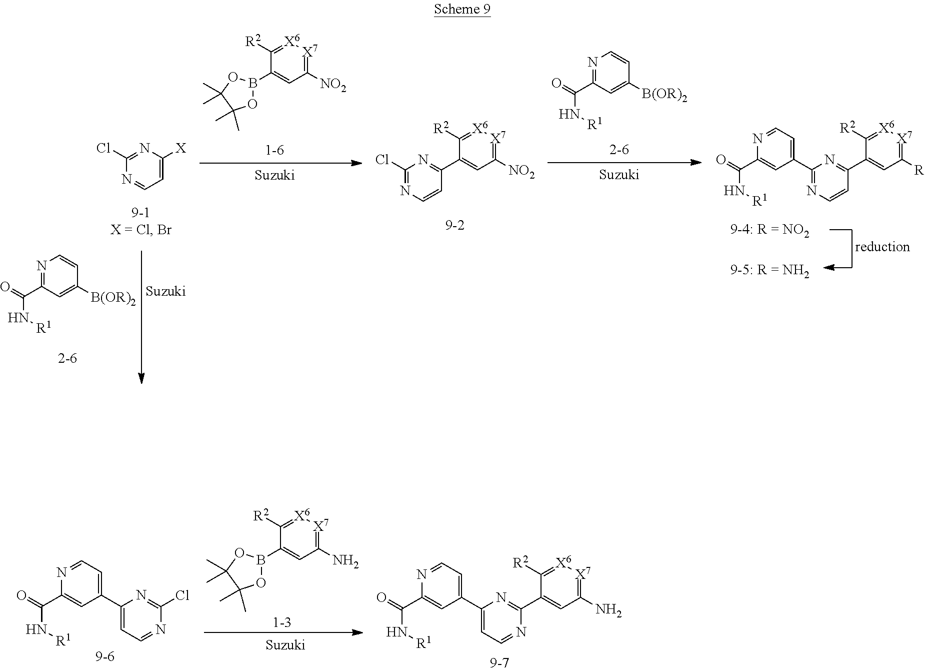

Scheme 9 illustrates an exemplary preparation of intermediates 9-5 and 9-7. Compounds 9-1 (2,4-dichloropyrimidine or 4-bromo-2-chloropyrimidine) react with boronates 1-6 by Suzuki reaction to afford 9-2 which can be subjected to another Suzuki reaction with boronates or boronic acids 2-6 to afford 9-4. Mild reduction (zinc or iron metal with ammonium chloride) of 9-4 affords the corresponding amines 9-5. In a similar manner, compounds 9-1 react with boronates or boronic acids 2-6 by Suzuki reaction to afford 9-6 which can be subjected to another Suzuki reaction with boronates 1-3 to furnish compounds 9-7.

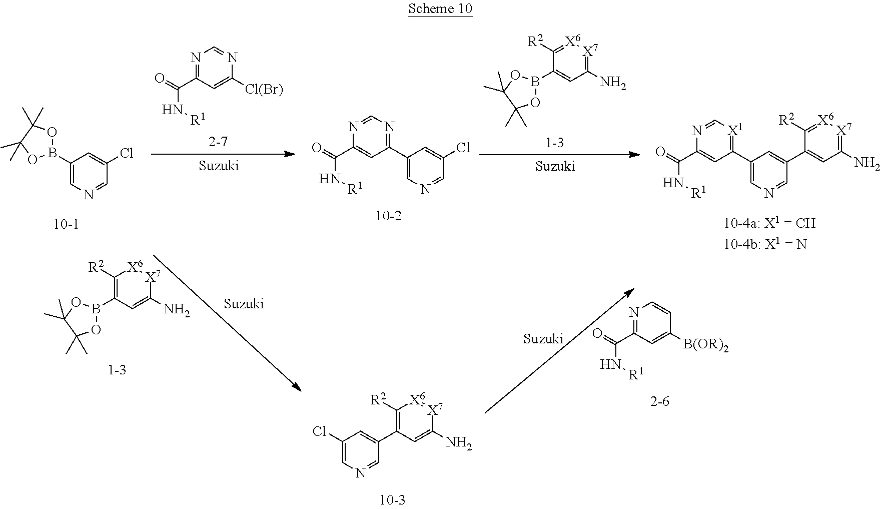

Scheme 10 illustrates an exemplary preparation of intermediates 10-4a and 10-4b. Treatment of 3-chloro-5-(4,4,5,5-tetramethyl-1,3,2-dioxaborolan-2-yl)pyri dine (10-1) with chlorides (or bromides) 2-7 in the presence of a palladium catalyst (Suzuki reaction) affords 10-2. Another Suzuki reaction of 10-2 with boronates 1-3 afford intermediates 10-4b (X¹=N). In a similar manner, chlorides 10-3 react with boronates or boronic acids 2-6 under a typical Suzuki condition to afford intermediates 10-4a (X¹=CH).

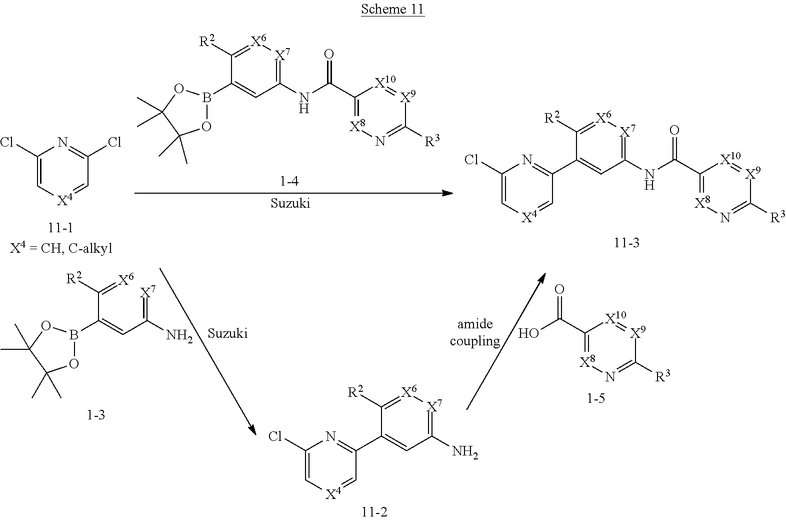

Scheme 11 illustrates an exemplary preparation of intermediates 11-3 (X⁴=CH, C-alkyl). Treatment of dichloropyridines 11-1 with boronates 1-3 in the presence of a palladium catalyst (Suzuki reaction) affords 11-2. Treatment of 11-2 with carboxylic acids 1-5 under amide coupling conditions known to those skilled in the art to afford 11-3. In another embodiment, commercially available 11-1 reacts with boronates 1-4 by Suzuki reaction to afford intermediates 11-3.

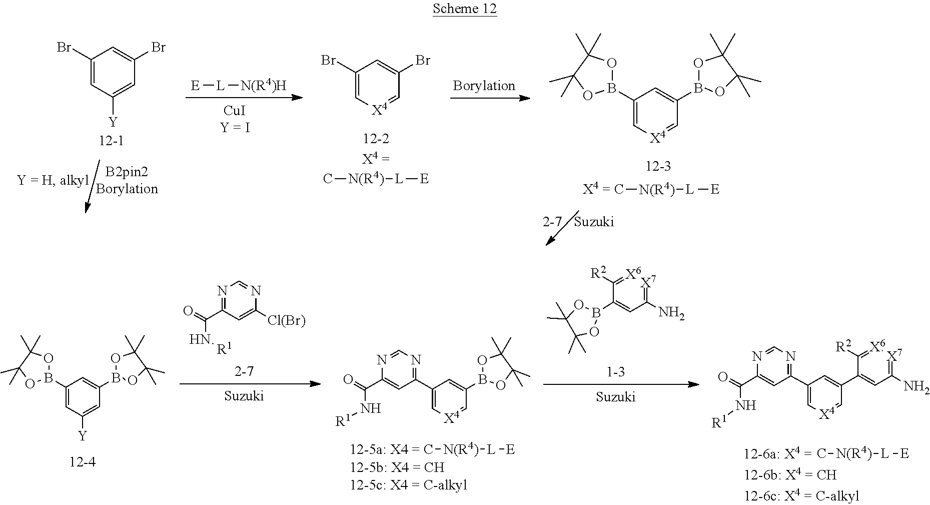

Scheme 12 illustrates an exemplary preparation of intermediates 12-6a, 12-6b, and 12-6c. 1,3-Dibromo-5-iodobenzene, 12-1 (Y═I) reacts with amines E-L-N(R⁴)H under the catalysis of CuI/L-proline in aprotic solvent such as DMSO in the presence of base such as K₂CO₃ at elevated temp to afford 12-2. A well-known borylation reaction of compounds 12-2 with B2pin2 under Pd (0) catalyst affords boronates 12-3 which can be reacted with chlorides (or bromides) 2-7 by Suzuki reaction to afford intermediates 12-5a (X⁴═C—N(R⁴)-L-E). In another embodiment, compounds 12-1 (Y═H, alkyl) react with B2pin2 under Pd (0) catalyst to afford boronates 12-4. Suzuki reaction of 12-4 with chlorides (or bromides) 2-7 (1 equivalent) affords 12-5b (X⁴═CH) and 12-5c (X⁴═C-alkyl) respectively. Finally, each compound 12-5a, 12-5b, and 12-5c reacts with boronates 1-3 under Suzuki reaction to afford 12-6a, 12-6b, and 12-6c respectively.

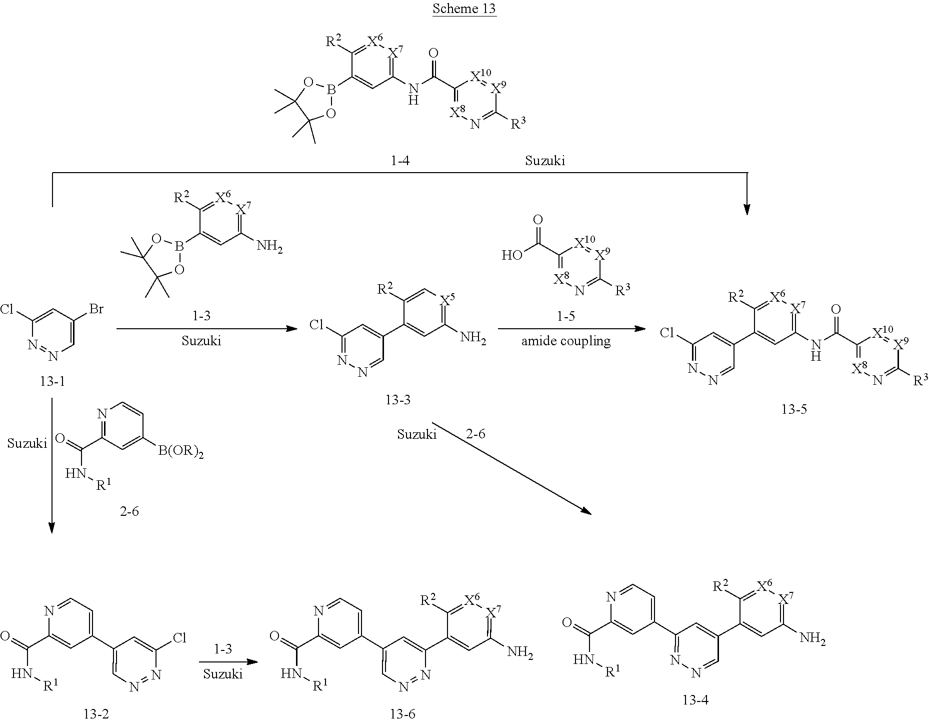

Scheme 13 illustrates an exemplary preparation of pyridazine intermediates 13-4, 13-5 and 13-6. Treatment of 5-bromo-3-chloropyridazine 13-1 with boronates 1-3 under Suzuki reaction conditions affords 13-3. Chlorides 13-3 react with boronates or boronic acids 2-6 to provide 13-4 by Pd (0) catalyzed Suzuki reaction. Chlorides 13-3 react with carboxylic acids 1-5 under amide coupling conditions to afford intermediates 13-5. Alternatively intermediates 13-5 can be directly prepared from 13-1 with boronates 1-4 by Suzuki reaction. Treatment of 5-bromo-3-chloropyridazine 13-1 with boronates or boronic acids 2-6 under Suzuki reaction conditions affords 13-2. Chlorides 13-2 react with boronates 1-3 to provide 13-6 by Pd (0) catalyzed Suzuki reaction.

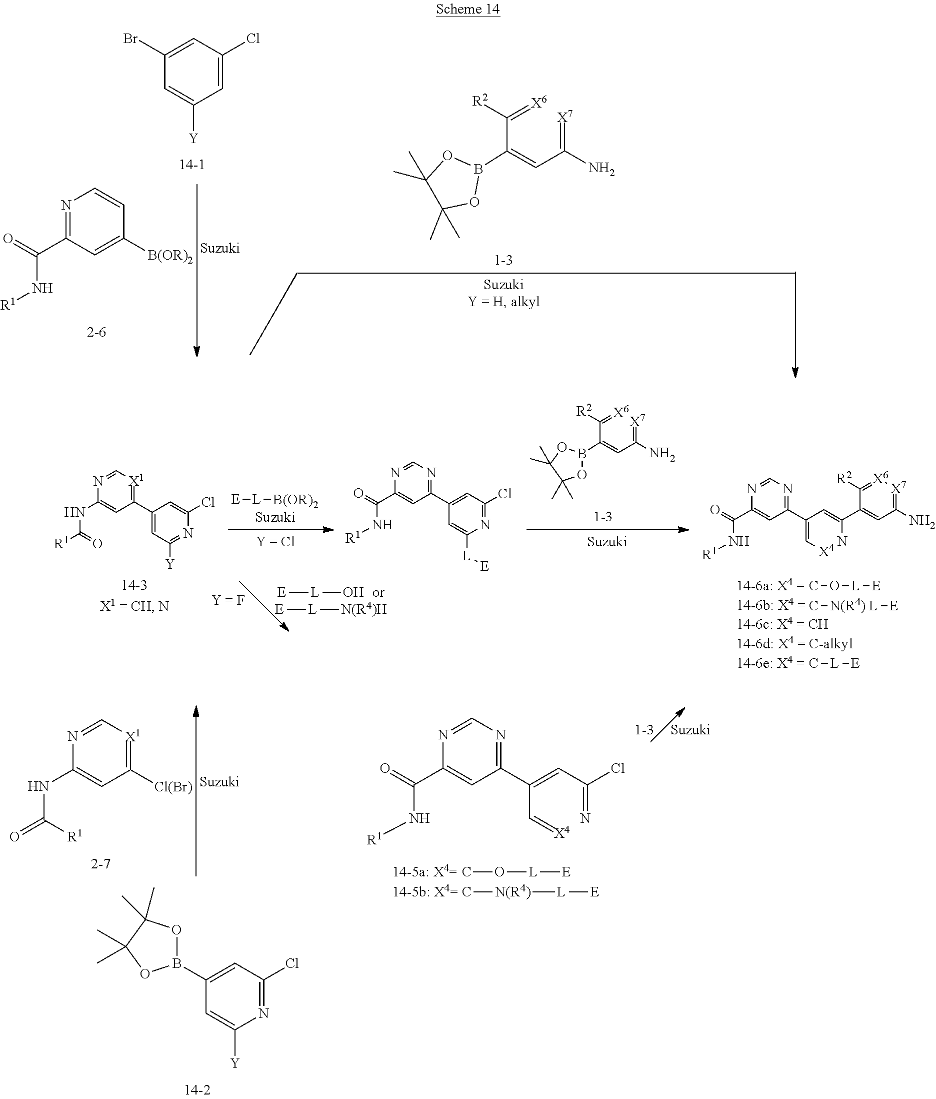

Scheme 14 illustrates an exemplary preparation of intermediates 14-6a, 14-6b, 14-6c, 14-6d, and 14-6e. Compounds 14-3 can be prepared by (1) Suzuki reaction of 14-1 (commercially available or synthesized by those skilled in the art) with boronic acids or boronates 2-6 or (2) Suzuki reaction of 14-2 (commercially available or synthesized by those skilled in the art) with chlorides (or bromides) 2-7 in the presence of a palladium catalyst. Compound 14-3 (Y═H, alkyl) reacts with boronates 1-3 by Suzuki reaction to afford 14-6c (X⁴═CH) and 14-6d (X⁴═C-alkyl), respectively. In another embodiment, compound 14-3 (Y═Cl) reacts with boronates or boronic acids E-L-B(OR)₂ under Pd (0) catalyzed coupling conditions using well known established literature conditions to those skilled in the art to afford 14-4. Suzuki reaction of 14-4 with boronates 1-3 under Pd (0) catalyzed coupling conditions affords 14-6e. If compounds 14-6e (X⁴=C-L-E) contain an unsaturated functionality such as a double bond, or a triple bond, hydrogenation can be proceeded in the presence of Pd catalyst. Alternatively, compounds 14-3 (Y=F) react with alcohols E-L-OH or amines E-L-N(R⁴)H by S_{N}Ar reaction in the presence of base to afford 14-5a (X⁴=C—O-L-E) and 14-5b (X⁴=C—N(R⁴)-L-E), respectively. Finally, chlorides 14-5a, and 14-5b can be reacted with boronates 1-3 under Suzuki reaction conditions to afford intermediates 14-6a (X⁴=C—O-L-E), and 14-6b (X⁴=C—N(R⁴)-L-E), respectively.

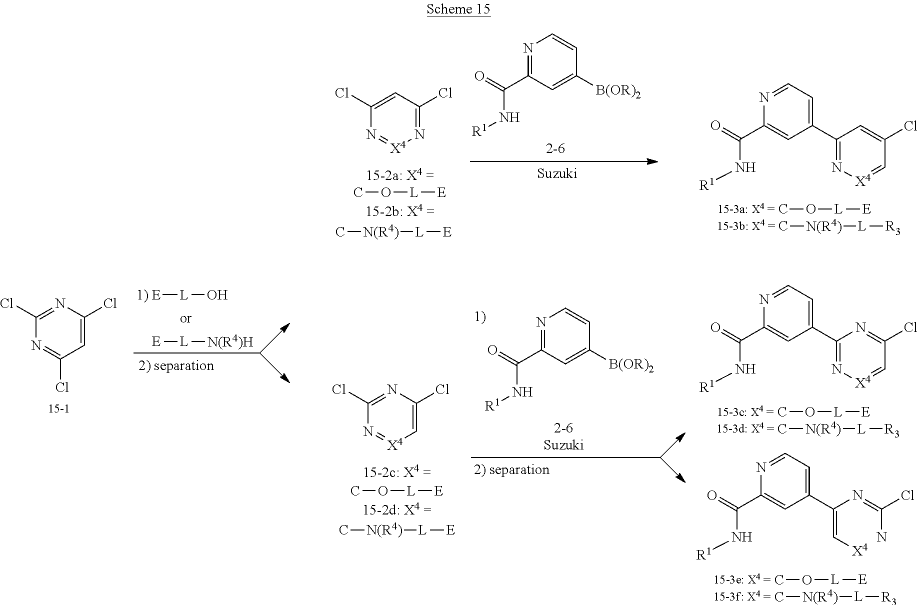

Scheme 15 illustrates an exemplary preparation of pyrimidine intermediates 15-3a, 15-3b, 15-3c, and 15-3d. S_{N}Ar reaction of 2,4,6-trichloropyrimidine 15-1 with alcohols (E-L-OH) affords a mixture of 15-2a (X⁴=C—O-L-E) and 15-2c (X⁴=C—O-L-E), which can be separated by a suitable method (for example: SFC purification, column chromatography or recrystallization). In a similar manner, 2,4,6-trichloropyrimidine 15-1 reacts with amines (E-L-N(R⁴)H) under S_{N}Ar reaction conditions to afford a mixture of 15-2b (X⁴=C—N(R⁴)-L-E), and 15-2d (X⁴=C—N(R⁴)-L-E), which can be separated by a suitable method (for example: SFC purification, column chromatography or recrystallization). Suzuki reaction of each 15-2a and 15-2b with boronates or boronic acids 2-6 affords 15-3a and 15-3b, respectively. Suzuki reaction of 15-2c (X⁴=C—O-L-E) with boronates or boronic acids 2-6 affords a mixture of 15-3c and 15-3e, which can be separated by a suitable method (for example: SFC purification, column chromatography or recrystallization). In similar manner, Suzuki reaction of 15-2d (X⁴═C—N(R⁴)-L-E) with boronates or boronic acids 2-6 affords a mixture of 15-3d and 15-3f, which can be separated by a suitable method (for example: SFC purification, column chromatography or recrystallization).

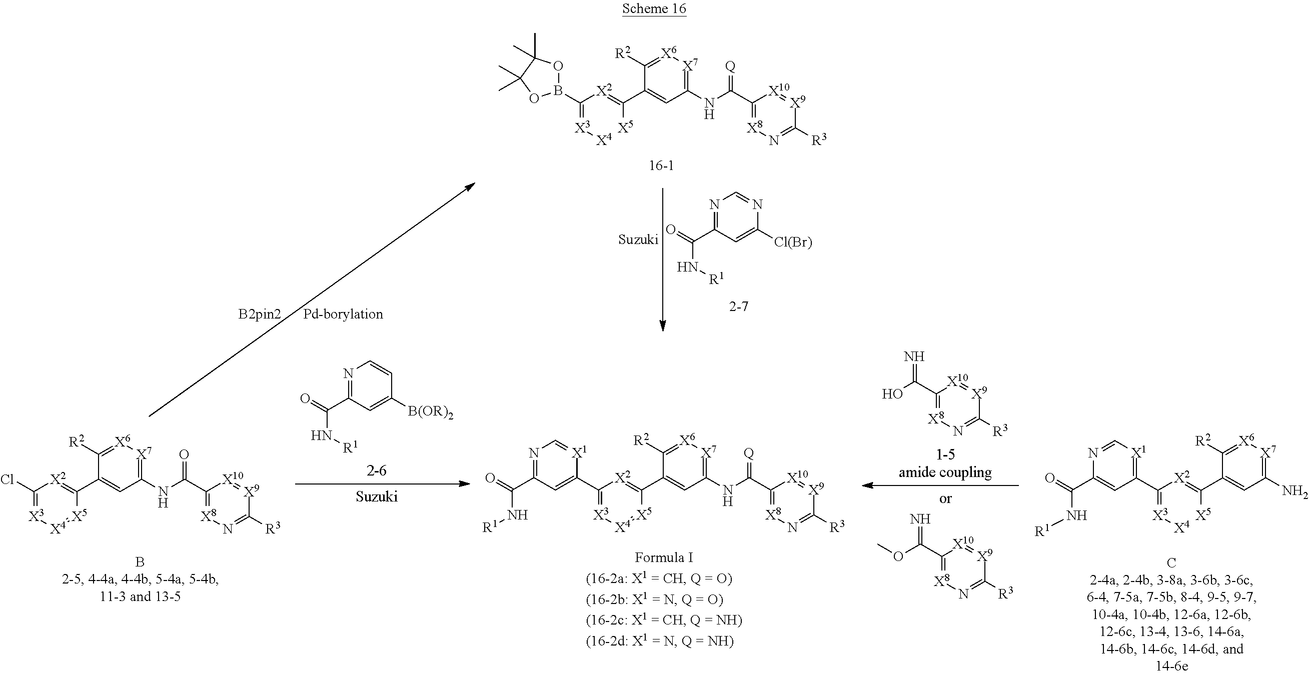

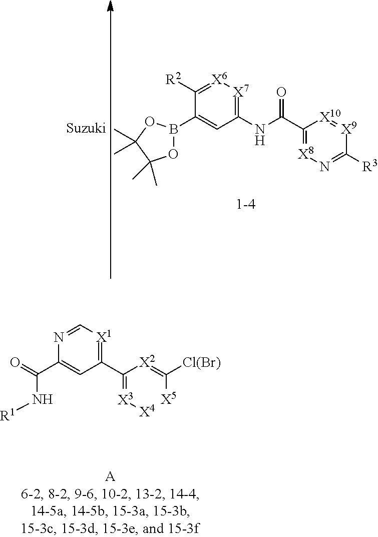

Scheme 16 illustrates an exemplary preparation of compounds of Formula I (16-2a, 16-2b, 16-2c, and 16-2d). Compounds of Formula I (16-2a and 16-2b) can be prepared by Suzuki reaction of chlorides A (6-2, 8-2, 9-6, 10-2, 13-2, 14-4, 14-5a, 14-5b, 15-3a, 15-3b, 15-3c, 15-3d, 15-3e, and 15-3f) with boronates 1-4. In a similar manner, chlorides B (2-5, 4-4a, 4-4b, 5-4a, 5-4b, 11-3, and 13-5) react with boronates or boronic acids 2-6 (commercially available or synthesized by those skilled in the art) under Suzuki condition to afford compounds of Formula I (16-2a). Alternatively, Pd-borylation of chlorides B (2-5, 4-4a, 4-4b, 5-4a, 5-4b, 11-3, and 13-5) with B2pin2 converts to boronates 16-1. Boronates 16-1 react with chlorides (or bromides) 2-7 (commercially available or synthesized by those skilled in the art) under Suzuki reaction conditions to afford compounds of Formula I (16-2b). In another embodiment, anilines C (2-4a, 2-4b, 3-6a, 3-6b, 3-6c, 6-4, 7-5a, 7-5b, 8-4, 9-5, 9-7, 10-4a, 10-4b, 12- 6a, 12-6b, 12-6c, 13-4, 13-6, 14-6a, 14-6b, 14-6c, 14-6d, and 14-6e) react with carboxylic acids 1-5 or imidates 1-7 under a typical coupling condition to convert compounds of Formula I (16-2a, 16-2b, 16-2c, and 16-2d) respectively.

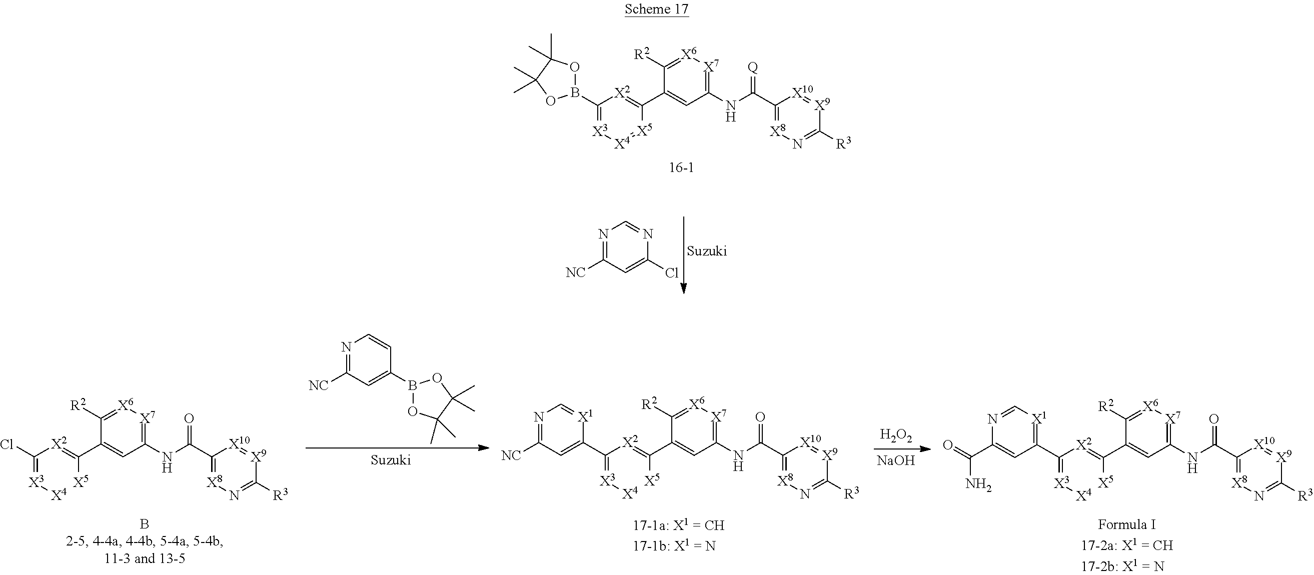

Scheme 17 illustrates an exemplary preparation of compounds of Formula I (17-2). Chlorides B (2-5, 4-4a, 4-4b, 5-4a, 5-4b, 11-3, and 13-5) react with 4-(4,4,5,5-tetramethyl-1,3,2-dioxaborolan-2-yl)picolinonitrile under Suzuki condition to afford 17-1a (X¹=CH). Alternatively, boronates 16-1 react with 6-chloropyrimidine-4-carbonitrile under Suzuki reaction conditions to afford 17-1b (X¹=N). Nitriles (17-1a and 17-1b) react with hydrogen peroxide in the presence of a small amount of alkali such as NaOH (well-known preparative procedure) to afford primary amides compounds of Formula I (17-2a, and 17-2b, respectively).

### PREPARATION OF INTERMEDIATES

Using the synthetic procedures and methods described herein and methods known to those skilled in the art, the following compounds were made:

### Preparation of Intermediate A1

### Intermediate A1: N-methyl-4-(4,4,5,5-tetramethyl-1,3,2-dioxaborolan-2-yl)picolinamide

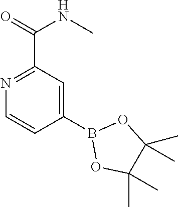 [SMILES: CNC(=O)c1cc(B2OC(C)(C)C(C)(C)O2)ccn1]

A mixture of 4-chloro-N-methylpicolinamide (3.5 g, 21 mmol), 4,4,4′,4′,5,5,5′,5′-octamethyl-2,2′-bi(1,3,2-dioxaborolane) (7.8 g, 31 mmol), and KOAc (6.0 g, 62 mmol) in 1,4-dioxane (70 mL) was purged with Ar for 2 min. PdCl₂(dppf) (0.75 g, 1.03 mmol) was added and the mixture was heated to 100° C. for 2.5 h. The reaction was cooled to rt and diluted with DCM (30 mL). The solution was filtered through a pad of celite and the filtrate was treated with sat'd NaHCO₃. The mixture was extracted with DCM (3×). The combined organics were dried over MgSO₄, filtered, and concentrated under reduced pressure. The residue was purified by silica gel column chromatography (0 to 20% MeOH/DCM) to obtain N-methyl-4-(4,4,5,5-tetramethyl-1,3,2-dioxaborolan-2-yl)picolinamide (3.1 g, 57%) as a tan solid. ¹H NMR (400 MHz, DMSO-d₆): δ 8.78 (d, J=5.6 Hz, 1H), 8.66 (d, J=4.7 Hz, 1H), 8.22 (s, 1H), 7.73 (dd, J=1.2, 4.7 Hz, 1H), 2.81 (d, J=4.9 Hz, 3H), 1.32 (s, 12H); MS (ESI) m/z: 181.2 (boronic acid, M+H⁺).

The following compounds are prepared essentially by method of preparation A1.

| Ex No | Product | Yield (%) | ¹H NMR (400 MHz, DMSO-d₆): □ | MS (m/z: M + H⁺) |
|---|---|---|---|---|
| A2 | 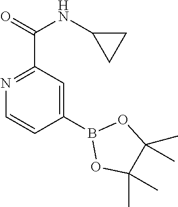 [SMILES: CC1(C)OB(c2ccnc(C(=O)NC3CC3)c2)OC1(C)C] | crude | 8.75 (d, J = 4.8 Hz, 1H), 8.65 (d, J = 4.4 Hz, 1H), 8.22 (s, 1H), 7.74 (d, J = 4.8 Hz, 1H), 2.91 (m, 1H), 1.33 (s, 12H), 0.64-0.70 (m, 4H). | 289.2 |

### Preparation of intermediate B1

### 3-(2-chloropyridin-4-yl)-4-methylaniline

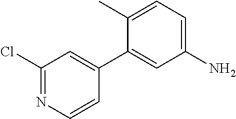

A solution of 4-bromo-2-chloropyridine (1.7 mL, 16 mmol) and 4-methyl-3-(4,4,5,5-tetramethyl-1,3,2-dioxaborolan-2-yl)aniline (5.4 g, 23 mmol), in 1,4-dioxane (70 mL) and water (8 mL) was treated with K₂CO₃ (5.4 g, 23 mmol). The reaction mixture was purged with nitrogen gas for 20 min then, Pd(dppf)Cl₂·DCM adduct (0.23 g, 0.31 mmol) was added. The reaction mixture was heated at 100° C. for 3.5 h and the mixture was cooled to rt. The solution was filtered through a pad of celite and washed with DCM (40 mL). The filtrate was washed with sat'd NaHCO₃, and water. The combined organics were dried over anhydrous MgSO₄, filtered, and concentrated under reduced pressure to obtain the crude. The crude was purified by silica gel column chromatography (0 to 100% EtOAc/hexanes) to obtain 3-(2-chloropyridin-4-yl)-4-methylaniline (3.4 g, 99%) as a light brown solid. ¹H NMR (400 MHz, DMSO-d₆): □□8.41 (d, J=5.1 Hz, 1H), 7.42 (d, J=1.4 Hz, 1H), 7.35 (dd, J=1.5, 5.1 Hz, 1H), 6.96 (d, J=8.2 Hz, 1H), 6.56 (dd, J=2.4, 8.1 Hz, 1H), 6.46 (d, J=2.4 Hz, 1H), 5.03 (s, 2H), 2.06 (s, 3H); MS (ESI) m/z: 219.2 (M+H⁺).

The following compounds are prepared essentially by method of preparation B1.

| Ex | | | Yield | ¹H NMR (400 MHz, | MS (m/z: |
|---|---|---|---|---|---|
| No | SM | Product | (%) | DMSO-d₆): □ | M + H+) |
| B2 | 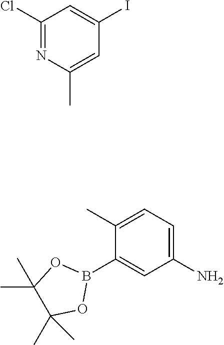 | 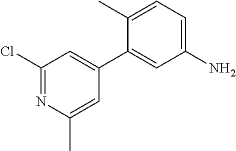 [SMILES: Cc1cc(-c2cc(N)ccc2C)cc(Cl)n1] | 93 | No Data | 233.2 |
| B3 | 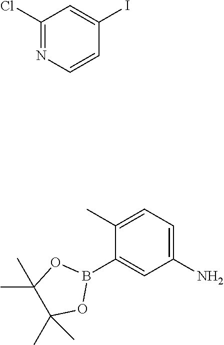 | 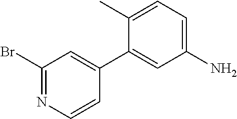 | 95 | 8.37 (d, J = 5.1 Hz, 1H), 7.53 (s, 1H), 7.37 (dd, J = 0.9, 5.0 Hz, 1H), 6.94 (d, J = 8.1 Hz, 1H), 6.54 (dd, J = 2.4, 8.1 Hz, 1H), 6.44 (d, J = 2.3 Hz, 1H), 5.01 (s, 2H), 2.04 (s, 3H). | 262.9 264.9 |
| B4 | 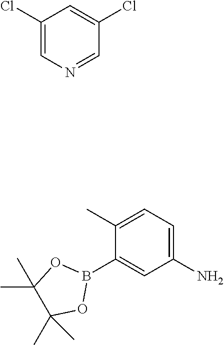 | 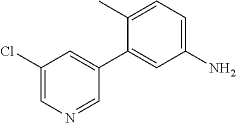 | 67 | 8.60 (s, 1H), 8.46 (s, 1H), 7.88 (s, 1H), 6.96 (d, J = 8.4 Hz, 1H), 6.55 (dd, J = 2.4, 8.4 Hz, 1H), 6.46 (d, J = 2.0 Hz, 1H), 5.09 (s, 2H), 2.05 (s, 3H). | 218.9 |
| B5 | 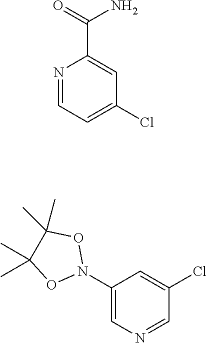 | 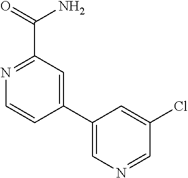 [SMILES: NC(=O)c1cc(-c2cncc(Cl)c2)ccn1] | 59 | No Data | 234.0 |
| B6 | 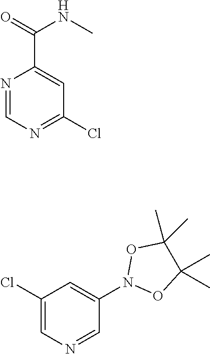 | 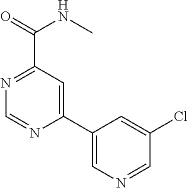 [SMILES: CNC(=O)c1cc(-c2cncc(Cl)c2)ncn1] | 71 | 9.42 (d, J = 1.3 Hz, 1H), 9.38 (d, J = 1.9 Hz, 1H), 9.08 (dd, J = 3.8, 8.6 Hz, 1H), 8.83 (d, J = 2.4 Hz, 1H), 8.75 (t, J = 2.2 Hz, 1H), 8.65 (d, J = 1.3 Hz, 1H), 2.86 (d, J = 4.8 Hz, 3H). | 249.0 |
| B7 | 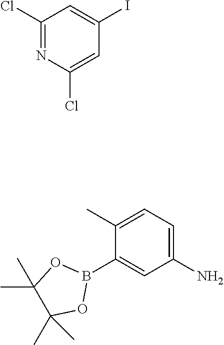 | 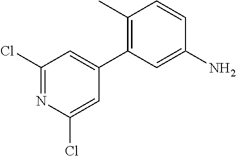 [SMILES: Cc1ccc(N)cc1-c1cc(Cl)nc(Cl)c1] | 67 | No Data | 253.0 |
| B8 | 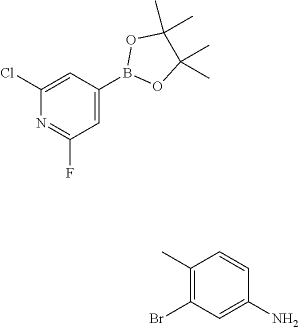 | 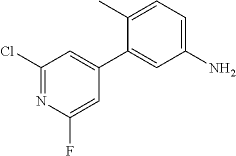 [SMILES: Cc1ccc(N)cc1-c1cc(F)nc(Cl)c1] | 65 | 7.44 (s, 1H), 7.21 (s, 1H), 6.97 (d, J = 8.0 Hz, 1H), 6.58 (dd, J = 2.4, 8.4 Hz, 1H), 6.48 (br s, 1H), 5.00 (br s, 2H), 2.08 (s, 3H). | 237.2 |
| B9 | 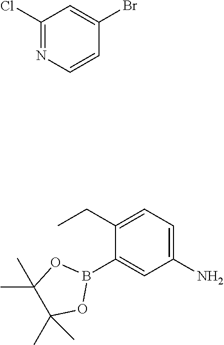 | 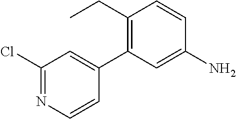 [SMILES: CCc1ccc(N)cc1-c1ccnc(Cl)c1] | 69 | No Data | 233.0 |
| B10 | 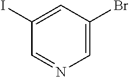 | 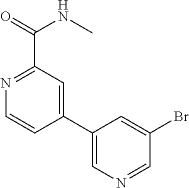 [SMILES: CNC(=O)c1cc(-c2cncc(Br)c2)ccn1] | 66 | 9.05 (d, J = 2.0 Hz, 1H), 8.84 (q, J = 4.8 Hz, 1H), 8.83 (d, J = 2.0 Hz, 1H), 8.75 (d, J = 4.8 Hz, 1H), 8.58 (t, J = 2.0 Hz, 1H), 8.35 (d, J = 1.2 Hz, 1H), 8.03 (dd, J = 2.0, 5.2 Hz, 1H), 2.85 (d, J = 4.8 Hz, 3H). | 292.0 |
| | A1 | | | | |
| B11 | 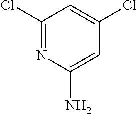 | 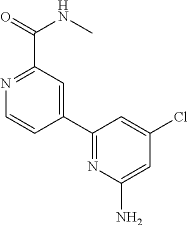 [SMILES: CNC(=O)c1cc(-c2cc(Cl)cc(N)n2)ccn1] | 43 | 8.84 (q, J = 4.2 Hz, 1H), 8.71 (d, J = 4.4 Hz, 1H), 8.65 (s, 1H), 8.15 (d, J = 8.0 Hz, 1H), 7.38 (s, 1H), 6.63 (s, 3H), 2.86 (d, J = 4.0 Hz, 3H). | 263.0 |
| | A1 | | | | |

### Preparation of Intermediate C1: 4-methyl-3-(5-(4,4,5,5-tetramethyl-1,3,2-dioxaborolan-2-yl)pyridin-3-yl)aniline

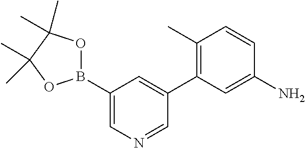 [SMILES: Cc1ccc(N)cc1-c1cncc(B2OC(C)(C)C(C)(C)O2)c1]

A mixture of 3-(5-chloropyridin-3-yl)-4-methyl aniline (B4, 1.0 g, 4.6 mmol), 4,4,4′,4′,5,5,5′,5′-octamethyl-2,2′-bi(1,3,2-dioxaborolane) (1.7 g, 6.8 mmol) and K₂CO₃ (1.4 g, 14 mmol) in 1,4-dioxane (12 mL) was purged with Ar for 5 min XPhos Pd G2 (0.18 g, 0.23 mmol) was added and the mixture was heated at 100° C. for 4 h. The mixture was cooled to rt and quenched with sat'd NaHCO₃ (aq) and EtOAc. The mixture was extracted with EtOAc (3×). The combined organic extracts were dried over anhydrous Na₂SO₄, filtered, and concentrated under reduced pressure to give 4-methyl-3-(5-(4,4,5,5-tetramethyl-1,3,2-dioxaborolan-2-yl)pyridin-3-yl)aniline (1.4 g, crude) as a brown solid. MS (ESI) m/z: 229.2 (boronic acid, M+H⁺).

### Preparation of intermediate D1 and D2: 2-((2,6-dichloropyrimidin-4-yl)amino)ethan-1-ol (D1) and 2-((4,6-dichloropyrimidin-2-yl)amino)ethan-1-ol (D2)

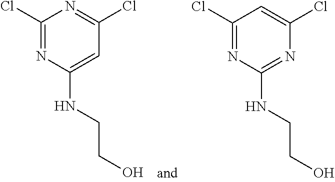 [SMILES: OCCNc1cc(Cl)nc(Cl)n1 ; OCCNc1nc(Cl)cc(Cl)n1]

A solution of 2-aminoethanol (3.3 mL, 54 mmol) in THF (30 mL) was added dropwise a solution of 2,4,6-trichloropyrimidine (3.1 mL, 27 mmol) in THF (50 mL). The mixture was stirred at 20° C. for 2 h and then concentrated under reduced pressure. The resulting crude was purified by silica gel column chromatography (0 to 20% EtOAc/pet-ether) to obtain 2-[(4,6-dichloropyrimidin-2-yl)amino]ethanol (D1, 2.0 g, 35%) as a white solid. ¹H NMR (400 MHz, DMSO-d₆): δ 8.25-8.33 (br m, 1H), 6.56 (s, 1H), 4.83 (t, J=8.0 Hz, 1H), 3.51 (m, 2H), 3.36 (m, 2H); MS (ESI) m/z: 208.0 (M+H⁺) and 2-[(2,6-dichloropyrimidin-4-yl)amino]ethanol (D2, 2.7 g, 48%) as a white solid. ¹H NMR (400 MHz, DMSO-d₆): δ 8.06 (t, J=5.6 Hz, 1H), 6.85 (s, 1H), 4.70 (t, J=5.6 Hz, 1H), 3.48 (m, 2H), 3.30 (m, 2H); MS (ESI) m/z: 208.0 (M+H⁺).

The following compounds are prepared essentially by method of preparation intermediate D1 and D2.

| Ex | | | Yield | | MS (m/z: |
|---|---|---|---|---|---|
| No | SM | Product | (%) | ¹H NMR (400 MHz, DMSO-d₆): □ | M + H+) |
| D3 | 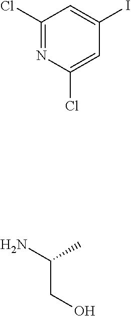 | 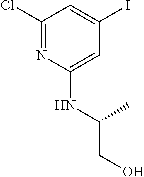 | 38 | 6.91 (d, J = 8.0 Hz, 1H), 6.85 (s, 1H), 6.82 (s, 1H), 4.72 (t, J = 5.6 Hz, 1H), 3.83 (m, 1H), 3.39 (m, 1H), 3.29 (m, 1H), 1.07 (d, J = 6.6 Hz, 3H). | 312.8 |
| D4 | 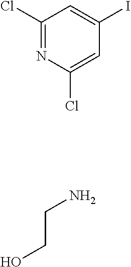 | 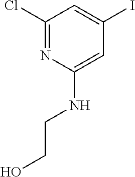 [SMILES: OCCNc1cc(I)cc(Cl)n1] | 58 | No Data | 299.0 |
| D5 | 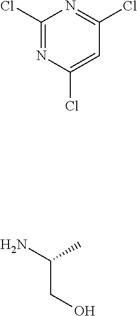 | 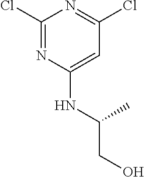 | 30 | 8.06 (d, J = 8.2 Hz, 1H), 6.49 (s, 1H), 4.82 (s, 1H), 4.01 (m, 1H), 3.35 (m, 2H), 1.08 (d, J = 6.7 Hz, 3H). | 222.0 |
| D6 | 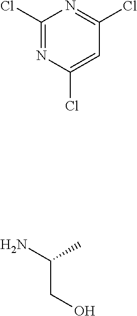 | 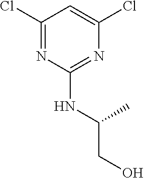 | 33 | 7.92 (d, J = 8.2 Hz, 1H), 6.83 (s, 1H), 4.69 (s, 1H), 3.90 (m, 1H), 3.40 (m, 1H), 3.30 (m, 1H), 1.09 (d, J = 6.8 Hz, 3H). | 222.0 |
| D7 | 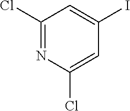 | 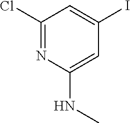 | 41 | 7.08 (q, J = 4.8 Hz, 1H), 6.87 (s, 1H), 6.81 (s, 1H), 2.72 (d, J = 4.8 Hz, 3H). | 269.0 |
| | CH₃NH₂ | | | | |
| D8 | 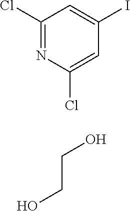 | 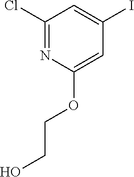 [SMILES: OCCOc1cc(I)cc(Cl)n1] | 67 | 7.56 (s, 1H), 7.34 (s, 1H), 4.90 (t, J = 5.5 Hz, 1H), 4.28-4.31 (m, 2H), 3.72-3.76 (m, 2H). | 300.1 |

### Preparation of Intermediate E1: 3-(2-(2-((tert-butyldimethylsilyl)oxy)ethoxy)-6-chloroyridin-4-1)-4-methylaniline

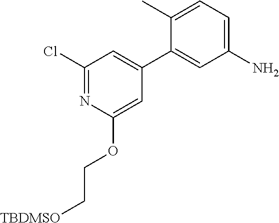 [SMILES: Cc1ccc(N)cc1-c1cc(Cl)nc(OCCO[Si](C)(C)C(C)(C)C)c1]

A solution of 2-((tert-butyldimethylsilyl)oxy)ethan-1-ol (0.42 g, 2.4 mmol) in DMF (5.0 mL) was treated with NaH (0.15 g, 3.8 mmol, 60 00 in mineral oil) slowly under an ice-water bath and the reaction mixture was stirred under the same conditions for 30 min. A solution of 3-(2-chloro-6-fluoropyridin-4-yl)-4-methylaniline (B8, 0.45 g, 1.9 mmol) in DMF (1 mL) was added into the reaction mixture and the reaction mixture was heated at 40° C. for 2 h. The reaction mixture was quenched with water (20 mL) under an ice-water bath. The solution was concentrated under reduced pressure and the residue was purified by silica gel column chromatography (5 to 10% EtOAc/hexanes) to obtain 3-(2-(2-((tert-butyldimethylsilyl)oxy)ethoxy)-6-chloropyridin-4-yl)-4-methylaniline (0.47 g, 62%). MS (ESI) m/z: 393.2 (M+H⁺).

The following compounds are prepared essentially by method of preparation intermediate E1.

| Ex | | | Yield | | MS (m/z: |
|---|---|---|---|---|---|
| No | SM | Product | (%) | ¹H NMR (400 MHz, DMSO-d₆): □ | M + H+) |
| E2 | 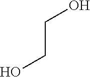 | 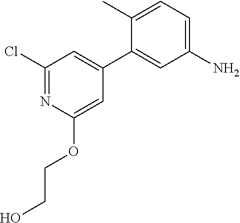 [SMILES: Cc1ccc(N)cc1-c1cc(Cl)nc(OCCO)c1] | 48 | 6.99 (d, J = 1.1 Hz, 1H), 6.95 (d, J = 8.2 Hz, 1H), 6.69 (d, J = 1.1 Hz, 1H), 6.55 (dd, J = 2.4, 8.1 Hz, 1H), 6.45 (d, J = 2.4 Hz, 1H), 5.04 (s, 2H), 4.86 (t, J = 5.5 Hz, 1H), 4.28 (t, J = 5.1 Hz, 2H), 3.72 (q, J = 5.0 Hz, 2H), 2.07 (s, 3H). | 279.0 |
| | B7 | | | | |
| | Cs₂CO₃ | | | | |

### Preparation of intermediate E3: (R)-2-((4-(5-amino-2-methylphenyl)-6-chloropyridin-2-yl)amino)propan-1-ol

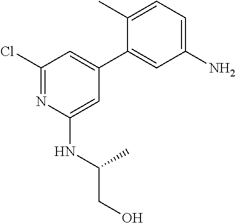 [SMILES: Cc1ccc(N)cc1-c1cc(Cl)nc(N[C@H](C)CO)c1]

A solution of (R)-2-((6-chloro-4-iodopyridin-2-yl)amino)propan-1-ol (D3, 2.7 g, 8.6 mmol) in a mixture of 1,4-dioxane (40 mL)/water (5 mL) was treated with K₂CO₃ (3.6 g, 26 mmol). 4-Methyl-3-(4,4,5,5-tetramethyl-1,3,2-dioxaborolan-2-yl)aniline (2.1 g, 9.1 mmol) was added and then the reaction mixture was degassed under Ar for 3 min. PdCl₂(dppf) (0.63 g, 0.86 mmol) was added, then the reaction mixture was heated overnight at 90° C. The reaction was cooled to rt and concentrated under reduced pressure. The residue was purified by silica gel column chromatography (1 to 100% MeOH/DCM) to obtain (R)-2-((4-(5-amino-2-methylphenyl)-6-chloropyridin-2-yl)amino)propan-1-ol (2.3 g, 86%) as a brown oil. ¹H NMR (400 MHz, DMSO-d₆): D D6.91 (d, J=8.0 Hz, 1H), 6.75 (d, J=7.9 Hz, 1H), 6.50 (dd, J=2.4, 8.2 Hz, 1H), 6.40 (d, J=2.4 Hz, 1H), 6.35 (d, J=0.8 Hz, 1H), 6.31 (d, J=0.8 Hz, 1H), 4.96 (s, 2H), 4.71 (t, J=5.6 Hz, 1H), 3.88 (m, 1H), 3.46 (m, 1H), 3.29 (m, 1H), 2.05 (s, 3H), 1.12 (d, J=6.6 Hz, 3H): 292.1

The following compounds are prepared essentially by method of preparation intermediate E3.

| Ex | | | Yield | | MS (m/z: |
|---|---|---|---|---|---|
| No | SM | Product | (%) | ¹H NMR (400 MHz, DMSO-d₆): □ | M + H+) |
| E4 | 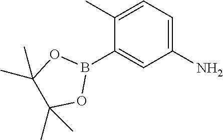 | 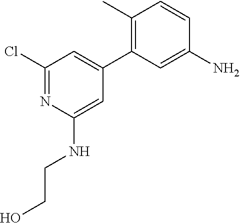 [SMILES: Cc1ccc(N)cc1-c1cc(Cl)nc(NCCO)c1] | 68 | 6.97 (t, J = 6.0 Hz, 1H), 6.91 (d, J = 9.2 Hz, 1H), 6.51 (br d, J = 8.8 Hz, 1H), 6.41 (br s, 1H), 6.38 (s, 1H), 6.34 (s, 1H), 4.92 (br s, 2H), 4.72 (t, J = 5.2 Hz, 1H), 3.54 (m, 2H), 3.31 (m, 2H), 2.05 (s, 3H). | 278.2 |
| | D4 | | | | |
| E5 | 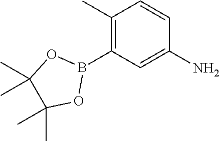 | 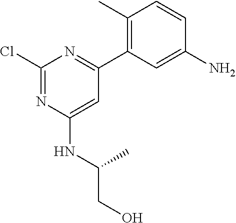 [SMILES: Cc1ccc(N)cc1-c1cc(N[C@H](C)CO)nc(Cl)n1] | 29 | 11.36 (s, 1H), 9.99 (d, J = 2.2 Hz, 1H), 8.60 (m, 1H), 8.10 (m, 1H), 7.92 (m, 2H), 7.36 (d, J = 8.4 Hz, 1H), 6.64 (s, 1H), 4.89 (t, J = 5.2 Hz, 1H), 4.16 (m, 1H), 3.48 (m, 2H), 2.38 (s, 3H), 1.19 (d, J = 6.9 Hz, 3H). | 467.2 |
| | D5 | | | | |
| E6 | 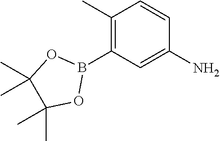 | 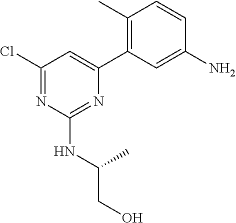 [SMILES: Cc1ccc(N)cc1-c1cc(Cl)nc(N[C@H](C)CO)n1] | 40 | 11.25 (s, 1H), 9.89 (d, J = 2.2 Hz, 1H), 8.52 (s, 1H), 8.30 (s, 1H), 7.84 (m, 1H), 7.56 (d, J = 7.7 Hz, 1H), 7.24 (d, J = 8.4 Hz, 1H), 6.43 (s, 1H), 4.74 (t, J = 5.5 Hz, 1H), 4.12 (s, 1H), 3.37 (m, 2H), 2.43 (s, 3H), 1.10 (d, J = 6.9 Hz, 3H). | 467.2 |
| | D6 | | | | |

### Preparation of Intermediate F1: 4-(5-amino-2-methylphenyl)-N-methyl-[2,4′-bipyridine]-2′-carboxamide

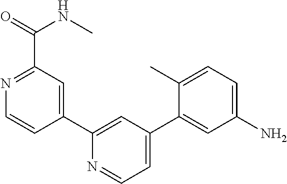 [SMILES: CNC(=O)c1cc(-c2cc(-c3cc(N)ccc3C)ccn2)ccn1]

A mixture of 3-(2-chloropyridin-4-yl)-4-methylaniline (B1, 2.5 g, 11 mmol), N-methyl-4-(4,4,5,5-tetramethyl-1,3,2-dioxaborolan-2-yl)picolinamide (A1, 4.5 g, 17 mmol) and K₂CO₃ (4.7 g, 34 mmol) in a mixture of 1,4-dioxane (80 mL) and water (9 mL) was purged with Ar for 10 min Pd(dppf)Cl₂·DCM adduct (0.93 g, 1.1 mmol) was added and then the reaction mixture was sealed and heated to 100° C. for 2 h. The reaction was cooled to rt and diluted with sat'd NaHCO₃ (aq). The mixture was extracted with DCM (3×) and the combined organics were dried over anhydrous Na₂SO₄, filtered, and concentrated under reduced pressure. The black oil was purified by silica gel column chromatography (0 to 8000 EtOAc/Hexanes) to obtain 4-(5-amino-2-methylphenyl)-N-methyl-[2,4′-bipyridine]-2′-carboxamide (2.3 g, 64%) as a white solid. ¹H NMR (400 _{N}Hz, DMSO-d₆): □□8.83 (q, J=5.5 Hz, 1H), 8.78 (d, J=5.0 Hz, 1H), 8.74 (m, 2H), 8.30 (dd, J=1.8, 5.1 Hz, 1H), 8.07 (s, 1H), 7.45 (dd, J=1.5, 5.0 Hz, 1H), 6.99 (d, J=8.1 Hz, 1H), 6.58 (dd, J=2.4, 8.1 Hz, 1H), 6.56 (d, J=2.4 Hz, 1H), 5.03 (s, 2H), 2.85 (d, J=4.8 Hz, 3H), 2.11 (s, 3H).

The following compounds are prepared essentially by method of preparation F1.

| Ex | | | Yield | | MS (m/z: |
|---|---|---|---|---|---|
| No | SM | Product | (%) | ¹H NMR (400 MHz, DMSO-d₆): □ | M + H⁺) |
| F2 | A1 B4 | 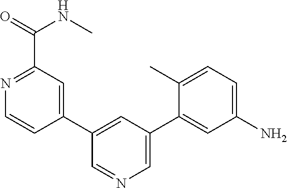 [SMILES: CNC(=O)c1cc(-c2cncc(-c3cc(N)ccc3C)c2)ccn1] | 66 | 9.01 (br t, J = 1.8 Hz, 1H), 8.85 (br q, J = 4.4 Hz, 1H), 8.72 (br d, J = 4.0 Hz, 1H), 8.63 (br s, 1H), 8.34 (br s, 1H), 8.16 (m, 1H), 8.04 (m, 1H), 6.99 (d, J = 8.0 Hz, 1H), 6.55-6.61 (m, 2H), 5.02 (s, 2H), 2.85 (d, J = 4.4 Hz, 3H), 2.11 (s, 3H). | 319.2 |
| F3 | A1 B2 | 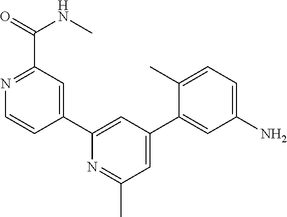 [SMILES: CNC(=O)c1cc(-c2cc(-c3cc(N)ccc3C)cc(C)n2)ccn1] | 47 | 8.83 (br q, J = 4.4 Hz, 1H), 8.74 (s, 1H), 8.71 (d, J = 6.0 Hz, 1H), 8.27 (br d, J = 4.8 Hz, 1H), 7.87 (s, 1H), 7.31 (s, 1H), 6.98 (d, J = 6.8 Hz, 1H), 6.58 (br d, J = 7.2 Hz, 1H), 6.55 (s, 1H), 5.01 (s, 2H), 2.86 (d, J = 4.8 Hz, 3H), 2.63 (s, 3H), 2.11 (s, 3H). | 333.2 |
| F4 | 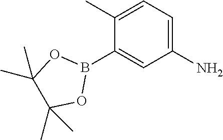 | 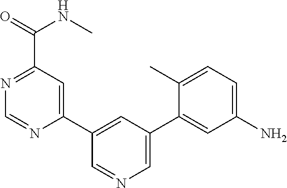 [SMILES: CNC(=O)c1cc(-c2cncc(-c3cc(N)ccc3C)c2)ncn1] | 97 | 9.40 (s, 1H), 9.37 (d, J = 2.1 Hz, 1H), 9.06 (q, J = 4.9 Hz, 1H), 8.70 (d, J = 2.1 Hz, 1H), 8.62 (s, 1H), 8.53-8.48 (m, 1H), 6.99 (d, J = 8.0 Hz, 1H), 6.60-6.53 (m, 2H), 5.02 (s, 2H), 2.86 (d, J = 4.8 Hz, 3H), 2.10 (s, 3H). | 320.2 |
| | B6 | | | | |
| F5 | A1 E4 | 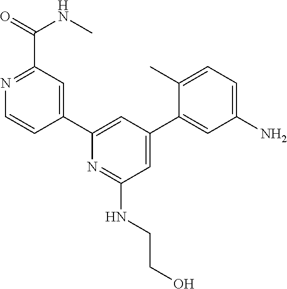 [SMILES: CNC(=O)c1cc(-c2cc(-c3cc(N)ccc3C)cc(NCCO)n2)ccn1] | 98 | No Data | 378.2 |
| F6 | 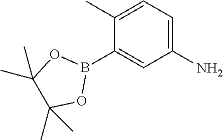 | 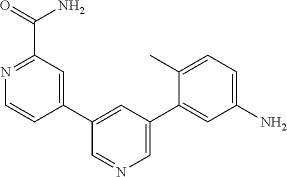 [SMILES: Cc1ccc(N)cc1-c1cncc(-c2ccnc(C(N)=O)c2)c1] | crude | No Data | 305.0 |
| | B5 | | | | |
| F7 | A1 E3 | 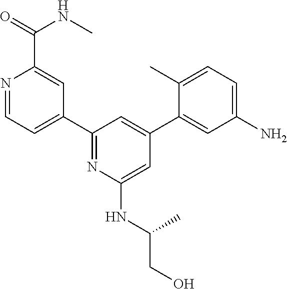 [SMILES: CNC(=O)c1cc(-c2cc(-c3cc(N)ccc3C)cc(N[C@H](C)CO)n2)ccn1] | 70 | 8.82 (q, J = 4.8 Hz, 1H), 8.68 (d, J = 5.2 Hz, 1H), 8.63 (s, 1H), 8.20 (dd, J = 1.6, 5.2 Hz, 1H), 7.14 (s, 1H), 6.96 (d, J = 8.0 Hz, 1H), 6.62 (d, J = 7.6 Hz, 1H), 6.51-6.56 (m, 3H), 5.00 (s, 2H), 4.77 (t, J = 5.4 Hz, 1H), 4.14 (m, 1H), 3.56 (m, 1H), 3.43 (m, 1H), 2.85 (d, J = 4.8 Hz, 3H), 2.12 (s, 3H), 1.22 (d, J = 6.8 Hz, 3H). | 392.2 |
| F8 | A1 E5 | 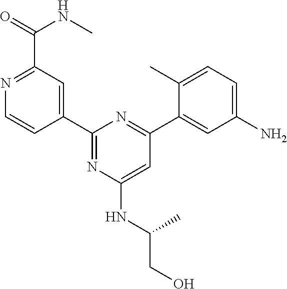 [SMILES: CNC(=O)c1cc(-c2nc(N[C@H](C)CO)cc(-c3cc(N)ccc3C)n2)ccn1] | crude | 11.33 (s, 1H), 9.96 (s, 1H), 8.81 (m, 3H), 8.59 (s, 1H), 8.29 (s, 1H), 8.15 (d, J = 2.3 Hz, 1H), 7.99 (d, J = 8.3 Hz, 1H), 7.44 (s, 1H), 7.37 (d, J = 8.4 Hz, 1H), 7.27 (s, 1H), 4.70 (s, 1H), 4.14 (s, 1H), 3.48 (m, 2H), 2.86 (d, J = 4.8 Hz, 3H), 2.45 (s, 3H), 1.21 (d, J = 6.6 Hz, 3H). | 467.2 |
| F9 | A1 E6 | 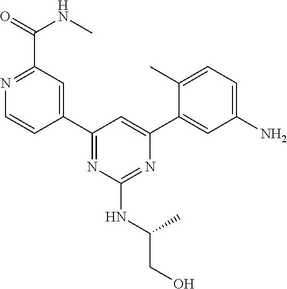 [SMILES: CNC(=O)c1cc(-c2cc(-c3cc(N)ccc3C)nc(N[C@H](C)CO)n2)ccn1] | 12 | No Data | 393.2 |
| F10 | 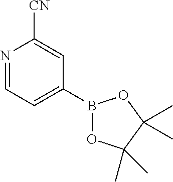 | 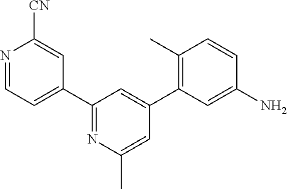 [SMILES: Cc1cc(-c2cc(N)ccc2C)cc(-c2ccnc(C#N)c2)n1] | 77 | No Data | 301.2 |
| | B2 | | | | |
| F11 | A1 B9 | 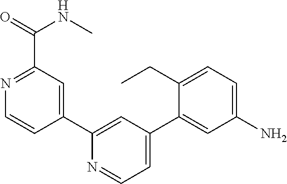 [SMILES: CCc1ccc(N)cc1-c1ccnc(-c2ccnc(C(=O)NC)c2)c1] | 44 | No Data | 333.2 |
| F12 | A1 E2 | 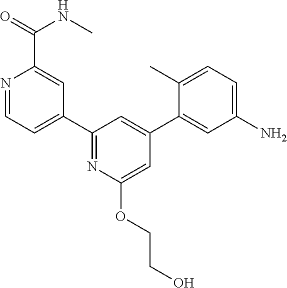 [SMILES: CNC(=O)c1cc(-c2cc(-c3cc(N)ccc3C)cc(OCCO)n2)ccn1] | 61 | 8.84 (q, J = 4.8 Hz, 1H), 8.73 (d, J = 5.1 Hz, 1H), 8.69 (s, 1H), 8.29 (br d, J = 5.0 Hz, 1H), 7.70 (s, 1H), 6.99 (d, J = 8.1 Hz, 1H), 6.83 (s, 1H), 6.53- 6.63 (m, 2H), 5.03 (s, 2H), 4.92 (t, J = 5.5 Hz, 1H), 4.48 (t, J = 5.0 Hz, 2H), 3.82 (q, J = 5.2 Hz, 2H), 2.86 (d, J = 4.8 Hz, 3H), 2.13 (s, 3H). | 379.2 |

### Preparation of Intermediate G1: 3-chloro-5-cyclopropylpyridazine

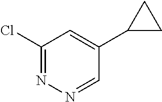

A suspension of 5-bromo-3-chloro-pyridazine (10 g, 52 mmol), cyclopropylboronic acid (4.4 g, 52 mmol), Na₂CO₃ (16 g, 155 mmol) in a mixture of 1,4-dioxane (100 mL) and H₂O (20 mL) was purged with N₂ for 3 min Pd(dppf)Cl₂ (3.0 g, 4.1 mmol) was added and then the mixture was heated at 90° C. for 12 h under N₂ atmosphere. The reaction mixture was cooled to rt, diluted with water (150 mL) and extracted with EtOAc (4×). The combined organic layers were washed with brine, dried over anhydrous Na₂SO₄, filtered, and concentrated under reduced pressure. The crude was purified by silica gel column chromatography (0 to 5000 EtOAc/pet-ether) to give 3-chloro-5-cyclopropylpyridazine (4.1 g, 51%) as a yellow solid. ¹H NMR (400 MHz, DMSO-d₆): δ 9.08 (d, J=2.0 Hz, 1H), 7.59 (d, J=1.9 Hz, 1H), 2.01 (m, 1H), 1.15-1.21 (m, 2H), 0.98-1.04 (in, 2H). MS (ESI) m/z: 155.1 (M+H⁺).

### Preparation of Intermediate G2: 3-chloro-5-(1-ethoxyvinyl)pyridazine

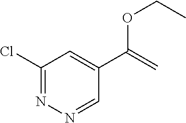

A 2 L three round-bottom flask equipped with a stirrer bar was charged with 5-bromo-3-chloro-pyridazine (40 g, 208 mmol), tributyl(1-ethoxyvinyl)stannane (70 mL, 208 mmol), TEA (87 mL, 620 mmol) in DMF (500 mL) was purged with N₂ for 3 min. Pd(PPh₃)₂Cl₂ (7.3 g, 10 mmol) was added and then the mixture was heated at 90° C. for 3 h under N₂ atmosphere. The reaction mixture was cooled to rt, quenched with sat'd NH₄Cl (100 mL) and sat'd KF(1 L) at 0° C. The mixture was stirred at rt for 0.5 h, then extracted with EtOAc (3×). The combined organic layers were washed by brine, dried over anhydrous Na₂SO₄, filtered, and concentrated under reduced pressure. The resulting crude was purified by silica gel column chromatography (0 to 100% EtOAc/pet-ether) to obtain 3-chloro-5-(1-ethoxyvinyl)pyridazine (30 g, 79%) as a yellow solid. ¹H NMR (400 MHz, methanol-d₄) δ 9.41 (d, J=1.6 Hz, 1H), 7.95 (d, J=1.8 Hz, 1H), 5.29 (d, J=3.8 Hz, 1H), 4.76 (d, J=3.9 Hz, 1H), 4.03 (q, J=7.0 Hz, 2H), 1.47 (t, J=7.0 Hz, 3H).

### Preparation of Intermediate H1: methyl 5-cyclopropylpyridazine-3-carboxylate

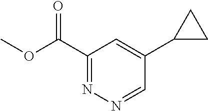

A solution of 3-chloro-5-cyclopropyl-pyridazine (4.0 g, 26 mmol) in MeOH (40 mL) was treated with DIEA (9.0 mL, 52 mmol). The suspension was purged with CO for 3 min and then Pd(dppf)Cl₂ (1.9 g, 2.6 mmol). The mixture was heated under CO (50 Psi) at 80° C. for 3 h. The reaction mixture was cooled to rt, diluted with water, and extracted with EtOAc (4×). The combined organic layers were washed with brine, dried over anhydrous Na₂SO₄, filtered, and concentrated under reduced pressure. The residue was purified by silica gel column chromatography (0 to 100% EtOAc/pet-ether) to give methyl 5-cyclopropylpyridazine-3-carboxylate (3.1 g, 67%) as a brown oil. ¹H NMR (400 MHz, DMSO-d₆): δ 9.23 (d, J=2.3 Hz, 1H), 7.83 (d, J=2.3 Hz, 1H), 3.94 (s, 3H), 2.10 (m, 1H), 1.13-1.25 (m, 2H), 0.95-1.06 (m, 2H); MS (ESI) m/z: 179.1 (M+H⁺).

The following compounds are prepared essentially by method of preparation H1.

| | | | Yield | | MS (m/z: |
|---|---|---|---|---|---|
| Ex No | SM | Product | (%) | ¹H NMR (400 MHz, DMSO-d₆): □ | M + H+) |
| H2 | 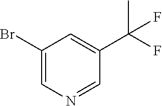 | 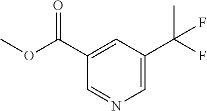 | 81 | 9.20 (s, 1H), 9.06 (s, 1H), 8.39 (s, 1H), 3.92 (s, 3H), 2.07 (t, J = 19.3 Hz, 3H) in methanol-d₄. | 202.0 |
| H3 | G2 and then HCl | 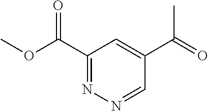 | 74 | 9.74 (d, J = 2.0 Hz, 1H), 8.62 (d, J = 2.1 Hz, 1H), 4.10 (s, 3H), 2.76 (s, 3H) in methanol-d₄. | No Data |
| H4 | 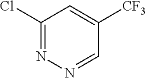 | 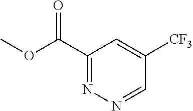 | 66 | 9.94 (d, J = 2.0 Hz, 1H), 8.53 (d, J = 1.8 Hz, 1H), 4.02 (s, 3H). | 207.1 |
| H5 | 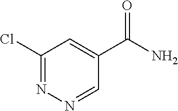 | 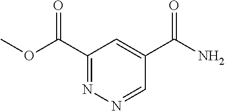 | 65 | 9.73 (d, J = 2.0 Hz, 1H), 8.64 (s, 1H), 8.54 (d, J = 2.2 Hz, 1H), 8.11 ( s, 1H), 4.00 (s, 3H). | 182.2 |

### Preparation of Intermediate 116: methyl 5-(trifluoromethyl)pyridazine-3-carbimidate

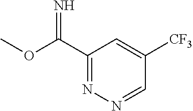

A solution of methyl 5-(trifluoromethyl)pyridazine-3-carb oxyl ate (1.5 g, 7.3 mmol) in 7.0 M NH₃/MeOH (10 mL) was stirred at 60° C. for 1 h. The reaction was cooled at rt, then concentrated under reduced pressure to give 5-(trifluoromethyl)pyridazine-3-carboxamide (1.2 g, 73%) as a yellow solid. ¹H NMR (400 MHz, DMSO-d₆): δ 9.89 (s, 1H), 8.81 (s, 1H), 8.47 (s, 1H), 8.20 (s, 1H). MS (ESI) m/z: 191.9 (M+H⁺).

A solution of 5-(trifluoromethyl)pyridazine-3-carboxamide (1.2 g, 6.3 mmol) in toluene (15 mL) was added POCl₃ (3.7 mL, 39 mmol) at rt. The reaction was heated at 100° C. for 1 h and then cooled to rt. The mixture was concentrated under reduced pressure. The resulting crude was purified by silica gel column chromatography (0 to 1000 EtOAc/pet-ether) to obtain 5-(trifluoromethyl)pyridazine-3-carbonitrile (0.8 g, 590%) as pale-yellow oil. ¹H NMR (400 MHz, CDCl₃): δ 9.57 (d, J=1.8 Hz, 1H), 8.02 (d, J=1.6 Hz, 1H); MS (ESI) m/z: 173.9 (M+H⁺).

A solution of 5-(trifluoromethyl)pyridazine-3-carbonitrile (0.2 g, 1.2 mmol) in MeOH (1 mL) was added NaOMe (0.46 g, 2.31 mmol). The reaction was stirred at rt for 1 h and concentrated under reduced pressure. The residue was diluted with water and extracted with EtOAc (2×). The combined extracts were washed by brine, dried over anhydrous Na₂SO₄, filtered, and concentrated under reduced pressure to obtain methyl 5-(trifluoromethyl)pyridazine-3-carbimidate (H6, 0.2 g, 84%) as a pale-yellow solid. ¹H NMR: (400 MHz, CDCl₃): δ 9.65 (s, 1H), 9.43 (d, J=1.8 Hz, 1H), 8.11 (d, J=1.8 Hz, 1H), 4.03 (s, 3H); MS (ESI) m/z: 206.0 (M+H⁺).

The following compounds are prepared essentially by method of preparation H6.

| Ex | | | Yield | ¹H NMR (400 MHz, | MS (m/z: |
|---|---|---|---|---|---|
| No | SM | Product | (%) | DMSO-d₆): □ | M + H⁺) |
| H7 | 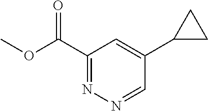 | 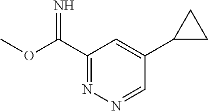 | 72 | No Data | 178.1 |

### Preparation of intermediate 118: methyl 5-(1,1-difluoroethyl)pyridazine-3-carboxylate

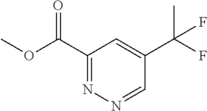

A solution of methyl 5-acetylpyridazine-3-carboxylate (H3, 30 g, 166 mmol) in DCM (300 mL) was stirred at 0° C. DAST (66 mL, 500 mmol) was added dropwise at 0° C. and warmed to rt. The reaction mixture was stirred at rt for 10 h. Water (300 mL) was added and then the solution was extracted with DCM (3×). The combined organic layers were washed with brine, dried over anhydrous Na₂SO₄, filtered, and concentrated under reduced pressure. The resulting crude was purified by silica gel column chromatography (0 to 100% EtOAc/pet-ether) to obtain methyl 5-(1,1-difluoroethyl)pyridazine-3-carboxylate (H8, 11 g, 33%) as a white solid. ¹H NMR (400 MHz, MeOD): δ 9.48 (d, J=2.2 Hz, 1H), 8.30 (d, J=2.0 Hz, 1H), 3.99 (s, 3H), 1.95 (t, J=18.9 Hz, 3H).

### Preparation of Intermediate H9: methyl 5-(2-cyanopropan-2-yl)nicotinate

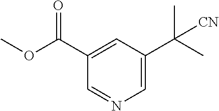

A mixture of methyl 5-bromopyridine-3-carboxylate (4.0 g, 18 mmol), 2-trimethylsilylacetonitrile (3.6 mL, 26 mmol), difluorozine (2.3 g, 22 mmol) in NMP (80 mL) was purged with N₂ for 3 min. Xantphos Pd G₄ (1.8 g, 1.8 mmol) was added, and then the mixture was heated at 130° C. for 3 h under N₂ atmosphere. The reaction mixture was diluted with H₂O (40 mL) and extracted with EtOAc (3×). The combined organic layers were washed with water, dried over anhydrous Na₂SO₄, filtered, and concentrated under reduced pressure. The crude was purified by silica gel column chromatography (0 to 100% EtOAc/pet-ether) to obtain methyl 5-(cyanomethyl)nicotinate (1.7 g, 52%) as a pink solid. ¹H NMR (400 MHz, DMSO-d₆): δ 9.05 (d, J=2.0 Hz, 1H), 8.82 (d, J=2.0 Hz, 1H), 8.31 (t, J=2.2 Hz, 1H), 4.23 (s, 2H), 3.92 (s, 3H); MS (ESI) m/z: 177.3 (M+H⁺).

A solution of methyl 5-(cyanomethyl)nicotinate (1.0 g, 5.7 mmol) in THF (10 mL) was added NaH (1.4 g, 34 mmol, 60% in mineral oil) at 0° C. The reaction mixture was stirred at 0° C. for 0.5 h under N₂ atmosphere, then Mel (2.1 mL, 34 mmol) was added at 0° C. and continuously stirred at 0° C. for 1 h under N₂ atmosphere. The reaction mixture was quenched by water and extracted with EtOAc (3×). The combined organic layers were washed with brine, dried over anhydrous Na₂SO₄, filtered, and concentrated under reduced pressure. The crude was purified by silica gel column chromatography (0 to 100% EtOAc/pet-ether) to obtain methyl 5-(2-cyanopropan-2-yl)nicotinate (119, 0.15 g, 13%) as a yellow solid. ¹H NMR (400 MHz, DMSO-d₆): δ 9.08 (d, J=2.0 Hz, 1H), 9.04 (d, J=2.4 Hz, 1H), 8.37 (t, J=2.2 Hz, 1H), 3.93 (s, 3H), 1.79 (s, 6H); MS (ESI) m/z: 205.0 (M+H⁺).

### Preparation of intermediate I1: 5-cyclopropylpyridazine-3-carboxylic acid

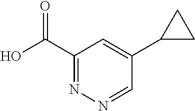 [SMILES: O=C(O)c1cc(C2CC2)cnn1]

A solution of methyl 5-cyclopropylpyridazine-3-carboxylate (111, 2.9 g, 16 mmol) in a mixture of THF (15 mL) and H₂O (15 mL) was treated with LiOH·H₂O (1.4 g, 32 mmol). The mixture was stirred at rt for 3 h and then concentrated under reduced pressure. The residue was acidified by 1.0 N HCl to pH around 3 and the resulting precipitate was collected by filtration to obtain 5-cyclopropylpyridazine-3-carboxylic acid (1.4 g, 52%) as a brown solid. ¹H NMR (400 MHz, DMSO-d₆): δ 9.22 (d, J=2.4 Hz, 1H), 7.82 (d, J=2.4 Hz, 1H), 2.10 (m, 1H), 1.13-1.25 (m, 2H), 0.97-1.06 (in, 2H), acid proton is missing; MS (ESI) m/z: 165.1 (M+H⁺).

The following compounds are prepared essentially by method of preparation I1.

| Ex | | | Yield | ¹H NMR (400 MHz, | MS (m/z: |
|---|---|---|---|---|---|
| No | SM | Product | (%) | DMSO-d₆): □ | M + H⁺) |
| I2 | H8 | 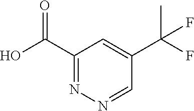 | 68 | 9.69 (d, J = 2.0 Hz, 1H), 8.28 (d, J = 2.0 Hz, 1H), 2.09 (t, J = 19.5 Hz, 3H), acid proton is missing. | 189.0 |
| I3 | H2 | 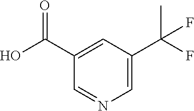 | 62 | 9.18 (s, 1H), 9.03 (s, 1H), 8.37 (s, 1H), 2.07 (t, J = 19.3 Hz, 3H), acid proton is missing. | 188.1 |
| I4 | H4 | 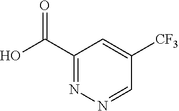 [SMILES: O=C(O)c1cc(C(F)(F)F)cnn1] | 95 | 9.63 (d, J = 2.0 Hz, 1H), 8.31 (d, J = 1.5 Hz, 1H), acid proton is missing. | 193.0 |
| I5 | H5 | 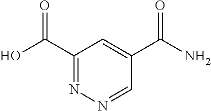 [SMILES: NC(=O)c1cnnc(C(=O)O)c1] | 36 | 9.54 (d, J = 2.2 Hz, 1H), 8.66 (s, 1H), 8.47 (d, J = 1.8 Hz, 1H), 7.97 (s, 1H), acid proton is missing. | 168.0 |

### Preparation of intermediate J1: N-(4-methyl-3-(4,4,5,5-tetramethyl-1,3,2-dioxaborolan-2-yl)phenyl)-5-(trifluoromethyl)pyridazine-3-carboxamide

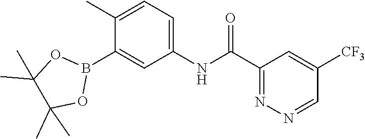 [SMILES: Cc1ccc(NC(=O)c2cc(C(F)(F)F)cnn2)cc1B1OC(C)(C)C(C)(C)O1]

A solution of 5-(trifluoromethyl)pyridazine-3-carboxylic acid (14, 6.35 g, 33 mmol), HATU (13.8 g, 36 mmol) and DIEA (17 mL, 99 mmol) in DMF (66 mL) was stirred at 0° C. for 15 min 4-methyl-3-(4,4,5,5-tetramethyl-1,3,2-dioxaborolan-2-yl)aniline (7.71 g, 33 mmol) was added and then the reaction was warmed to rt and stirred at rt overnight. The reaction mixture was quenched with water and the resulting solid was collected by filtration to obtain

N-(4-methyl-3-(4,4,5,5-tetramethyl-1,3,2-dioxaborolan-2-yl)phenyl)-5-(trifluoromethyl)pyridazine-3-carboxamide (12 g, 87%) as a white solid. ¹H NMR (400 MHz, DMSO-d₆): δ 11.14 (s, 1H), 9.92 (s, 1H), 8.55 (d, J=2.3 Hz, 1H), 8.20 (d, J=2.5 Hz, H), 7.85 (dd, J=2.4, 8.2 Hz, 1H), 7.19 (d, J=8.3 Hz, 1H), 2.44 (s, 3H), 1.30 (d, J=1.4 Hz, 12H); MS (ESI) m/z: 408.2 (M+H⁺).

The following compounds are prepared essentially by method of preparation intermediate J1.

| | | | | | MS |
|---|---|---|---|---|---|
| | | | | ¹H NMR | (m/z: |
| Ex | | | Yield | (400 MHz, | M + |
| No | SM | Product | (%) | DMSO-d₆): □ | H+) |
| J2 | 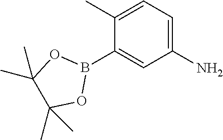 [SMILES: Cc1ccc(N)cc1B1OC(C)(C)C(C)(C)O1] | 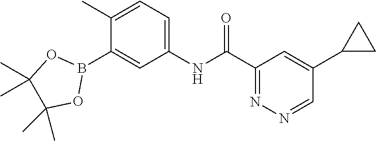 [SMILES: Cc1ccc(NC(=O)c2cc(C3CC3)cnn2)cc1B1OC(C)(C)C(C)(C)O1] | 74 | 10.91 (s, 1H), 9.26 (d, J = 2.4 Hz, 1H), 8.22 (d, J = 2.4 Hz, 1H), 7.91 (d, J = 2.4 Hz, 1H), 7.82 (dd, J = 2.4, 8.2 Hz, 1H), 7.17 (d, J = 8.4 Hz, 1H), 2.44 (s, 3H), 2.15 (m, 1H), 1.31 (s, 12H), 1.19-1.25 (m, 2H), 0.99- 1.06 (m, 2H). | 380.2 |
| | I1 | | | | |
| J3 | 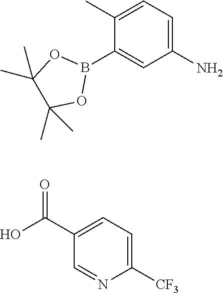 | 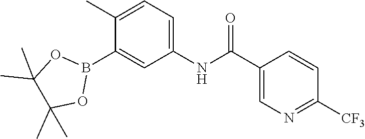 [SMILES: Cc1ccc(NC(=O)c2ccc(C(F)(F)F)nc2)cc1B1OC(C)(C)C(C)(C)O1] | 70 | No Data | 407.0 |
| J4 | 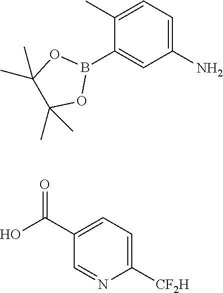 | 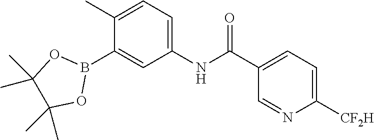 [SMILES: Cc1ccc(NC(=O)c2ccc(C(F)F)nc2)cc1B1OC(C)(C)C(C)(C)O1] | 66 | No Data | 389.0 |
| J5 | 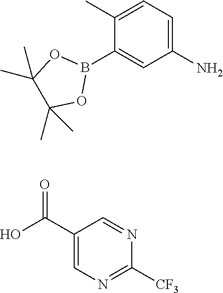 | 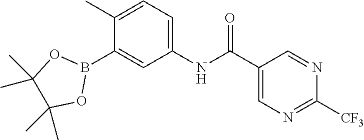 [SMILES: Cc1ccc(NC(=O)c2cnc(C(F)(F)F)nc2)cc1B1OC(C)(C)C(C)(C)O1] | 64 | 10.71 (s, 1H), 9.52 (s, 2H), 7.95 (d, J = 2.1 Hz, 1H), 7.90 (dd, J = 2.2, 8.4 Hz, 1H), 7.22 (d, J = 8.4 Hz, 1H), 2.46 (s, 3H), 1.32 (s, 12H). | 407.9 |
| J6 | 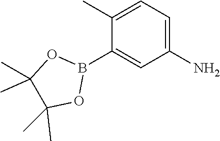 [SMILES: Cc1ccc(N)cc1B1OC(C)(C)C(C)(C)O1] | 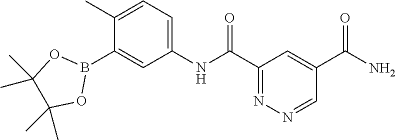 [SMILES: Cc1ccc(NC(=O)c2cc(C(N)=O)cnn2)cc1B1OC(C)(C)C(C)(C)O1] | 63 | 11.08 (s, 1H), 9.73 (d, J = 2.0 Hz, 1H), 8.59- 8.69 (m, 2H), 8.22 (d, J = 2.0 Hz, 1H), 8.10 (s, 1H), 7.86 (dd, J = 2.4, 8.2 Hz, 1H), 7.20 (d, J = 8.4 Hz, 1H), 2.45 (s, 3H), 1.32 (s, 12H). | 383.2 |
| | I5 | | | | |

### Preparation of intermediate J7: 5-cyano-N-(4-methyl-3-(4,4,5,5-tetramethyl-1,3,2-dioxaborolan-2-yl)phenyl)pyridazine-3-carboxamide

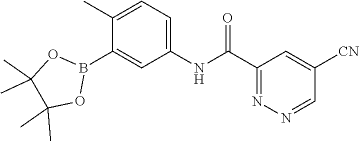 [SMILES: Cc1ccc(NC(=O)c2cc(C#N)cnn2)cc1B1OC(C)(C)C(C)(C)O1]

A solution of N3-[4-methyl-3-(4,4,5,5-tetramethyl-1,3,2-dioxaborolan-2-yl)phenyl]pyridazine-3,5-dicarboxamide (J6, 16.5 g, 43 mmol) in THF (200 mL) was treated with methoxycarbonyl-(triethylammonio)sulfonyl-azanide (51.4 g, 216 mmol). The mixture was stirred at 20° C. for 1 h, then the resulting precipitate was collected by filtration to give 5-cyano-N-[4-methyl-3-(4,4,5,5-tetramethyl-1,3,2dioxaborolan-2-yl)phenyl]pyridazine-3-carboxamide (5.0 g, 31%) as a yellow solid. The mother liquor was concentrated under reduced pressure and then the resulting residue was triturated with water (50 mL). The solid was filtered to obtain second batch of 5-cyano-N-[4-methyl-3-(4,4,5,5-tetramethyl-1,3,2dioxaborolan-2-yl)phenyl]pyridazine-3-carboxamide (4.2 g, 25%) as a yellow solid. ¹H NMR (400 MHz, DMSO-d₆): δ 11.13 (s, 1H), 9.86 (d, J=1.8 Hz, 1H), 8.78 (d, J=1.8 Hz, 1H), 8.21 (d, J=2.2 Hz, 1H), 7.85 (dd, J=2.2, 8.2 Hz, 1H), 7.20 (d, J=8.2 Hz, 1H), 2.45 (s, 3H), 1.32 (s, 12H); MS (ESI) m/z: 365.1 (M+H⁺).

### Preparation of intermediate K1: N-(3-(2-chloropyridin-4-yl)-4-methylphenyl)-6-(difluoromethyl)nicotinamide

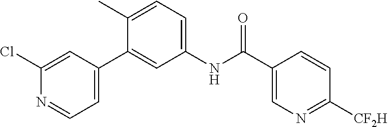 [SMILES: Cc1ccc(NC(=O)c2ccc(C(F)F)nc2)cc1-c1ccnc(Cl)c1]

A solution of 6-(difluoromethyl)nicotinic acid (0.40 g, 2.3 mmol) in DMF (6 mL) was treated with HATU (0.87 g, 2.3 mmol) and DIEA (0.6 mL, 3.4 mmol). The mixture was stirred at rt for 10 min. A solution of 3-(2-chloropyridin-4-yl)-4-methylaniline (B1, 0.25 g, 1.1 mmol) was added and the reaction mixture was stirred at rt for 4 h. The reaction mixture was quenched with ice water and allowed to stir for 90 min. The yellow solid was filtered and dried under high vacuum. The yellow solid was purified by silica gel column chromatography (0 to 50% EtOAc/hexanes) to afford N-(3-(2-chloropyridin-4-yl)-4-methylphenyl)-6-(difluoromethyl)nicotinamide (0.30 g, 70%) as a yellow solid. ¹H NMR (400 MHz, DMSO-d₆): δ 10.63 (s, 1H), 9.19 (s, 1H), 8.48-8.51 (m, 2H), 7.89 (d, J=8.1 Hz, 1H), 7.77 (d, J=8.3 Hz, 1H), 7.74 (s, 1H), 7.56 (s, 1H), 7.48 (d, J=5.1 Hz, 1H), 7.38 (d, J=8.3 Hz, 1H), 7.04 (t, J=56.0 Hz, 1H), 2.26 (s, 3H); MS (ESI) m/z: 374.2 (M+H⁺).

The following compounds are prepared essentially by method of preparation intermediate K1.

| Ex | | | Yield | MS (m/z: |
|---|---|---|---|---|
| No | SM | Product | (%) | M + H⁺) |
| K8 | B1 I4 | 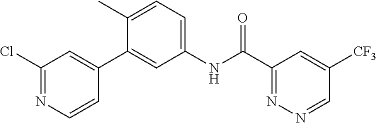 [SMILES: Cc1ccc(NC(=O)c2cc(C(F)(F)F)cnn2)cc1-c1ccnc(Cl)c1] | 98 | 393.0 |
| K9 | E2 I1 | 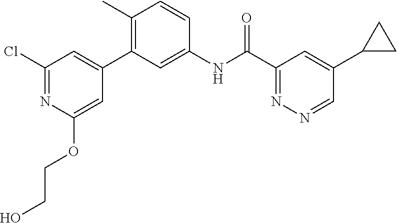 [SMILES: Cc1ccc(NC(=O)c2cc(C3CC3)cnn2)cc1-c1cc(Cl)nc(OCCO)c1] | 75 | 425.2 |
| K10 | B7 I4 | 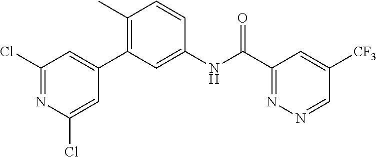 [SMILES: Cc1ccc(NC(=O)c2cc(C(F)(F)F)cnn2)cc1-c1cc(Cl)nc(Cl)c1] | 64 | 426.0 |
| K15 | B1 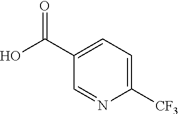 | 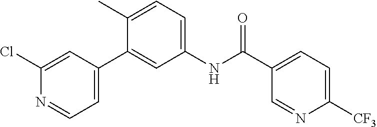 [SMILES: Cc1ccc(NC(=O)c2ccc(C(F)(F)F)nc2)cc1-c1ccnc(Cl)c1] | crude | 392.0 |

### Preparation of Intermediate K2: N-[3-[6-chloro-2-(2-hydroxyethylamino)pyrimidin-4-yl]-4-methyl-phenyl]-5-(trifluoromethyl)pyridazine-3-carboxamide

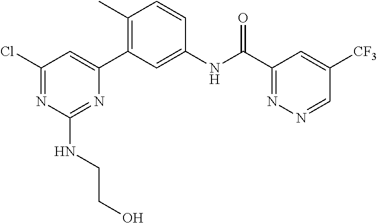 [SMILES: Cc1ccc(NC(=O)c2cc(C(F)(F)F)cnn2)cc1-c1cc(Cl)nc(NCCO)n1]

A mixture of 2-[(4,6-dichloropyrimidin-2-yl)amino]ethanol (D2, 0.20 g, 0.96 mmol), and N-[4-methyl-3-(4,4,5,5-tetramethyl-1,3,2-dioxaborolan-2-yl)phenyl]-5-(trifluoromethyl)pyridazine-3-carboxamide (J1, 0.39 g, 0.96 mmol) in 1,4-dioxane (3 mL) was treated with 2.0 M Na₂CO₃ (1.5 mL, 3 eq). The mixture was purged with N₂ for 3 min, and then Pd(dppf)Cl₂ (0.070 g, 0.1 eq) was added. The reaction mixture was heated at 90° C. for 2 h under N₂ atmosphere. The reaction mixture was diluted with water and extracted with EtOAc (2×). The combined organic layers were washed with brine, dried over anhydrous Na₂SO₄, filtered, and concentrated under reduced pressure. The resulting crude was purified by silica gel column chromatography (0 to 6500 EtOAc/pet-ether) to obtain N-[3-[6-chloro-2-(2-hydroxyethylamino)pyrimidin-4-yl]-4-methyl-phenyl]-5-(trifluoromethyl)pyridazine-3-carboxamide (0.20 g, 46%) as a brown solid. MS (ESI) m/z: 452.9 (M+H⁺).

The following compounds are prepared essentially by method of preparation intermediate K2.

| | | | | | MS |
|---|---|---|---|---|---|
| | | | | | (m/z: |
| Ex | | | Yield | ¹H NMR (400 MHz, | M + |
| No | SM | Product | (%) | DMSO-d₆): □ | H⁺) |
| K3 | D1 J2 | 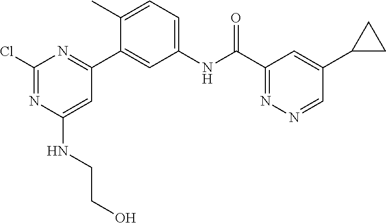 [SMILES: Cc1ccc(NC(=O)c2cc(C3CC3)cnn2)cc1-c1cc(NCCO)nc(Cl)n1] | 9 | 11.06 (s, 1H), 9.26 (s, 1H), 8.03 (br m, 2H), 7.92 (s, 1H), 7.86 ( d, J = 8.4 Hz, 1H), 7.30 (d, J = 8.4 Hz, 1H), 6.61 (s, 1H), 4.83 (br m, 1H), 3.56 (br m, 2H), 3.36-3.47 (m, 2H), 2.32 (s, 3H), 2.15 (m, 1H), 1.17- 1.29 (m, 2H), 0.98-1.06 (m, 2H). | 425.1 |
| K4 | D1 J1 | 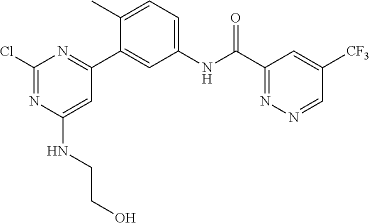 [SMILES: Cc1ccc(NC(=O)c2cc(C(F)(F)F)cnn2)cc1-c1cc(NCCO)nc(Cl)n1] | 9 | 11.34 (s, 1H), 9.96 (d, J = 1.6 Hz, 1H), 8.57 (s, 1H), 8.00-8.15 (m, 2H), 7.89 (d, J = 8.0 Hz, 1H), 7.33 (d, J = 8.4 Hz, 1H), 6.62 (s, 1H), 5.00-6.00 (br s, 1H), 3.57 (m, 2H), 3.42 (m, 2H), 2.33 (s, 3H). | 453.1 |
| K5 | D2 J2 | 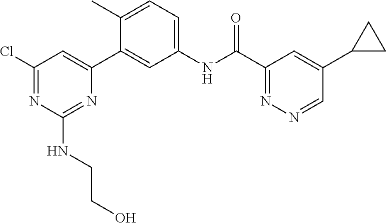 [SMILES: Cc1ccc(NC(=O)c2cc(C3CC3)cnn2)cc1-c1cc(Cl)nc(NCCO)n1] | 56 | 11.07 (s, 1H), 9.27 (d, J = 1.8 Hz, 1H), 8.10 (s, 1H), 7.92-7.94 (m, 2H), 7.68 (br s, 1H), 7.30 (d, J = 8.4 Hz, 1H), 6.80 (s, 1H), 4.74 (br s, 1H), 3.52 (br m, 2H), 2.38 (s, 3H), 2.15 (m, 1H), 1.12-1.31 (m, 2H), 0.99-1.10 (m, 2H), 2Hs are under solvents. | 425.0 |
| K6 | D1 J1 | 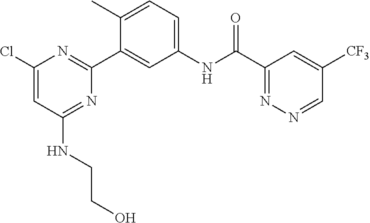 [SMILES: Cc1ccc(NC(=O)c2cc(C(F)(F)F)cnn2)cc1-c1nc(Cl)cc(NCCO)n1] | 25 | 9.74 (d, J = 1.8 Hz, 1H), 8.65 (s, 1H), 8.16 (s, 1H), 7.88 (dd, J = 2.2, 8.2 Hz, 1H), 7.35 (d, J = 8.4 Hz, 1H), 6.43 -6.68 (br m, 1H), 3.71- 3.76 (m, 2H), 3.62 (br m, 2H), 2.50 (s, 3H) in MeOD. | 453.1 |
| K7 | D1 J2 | 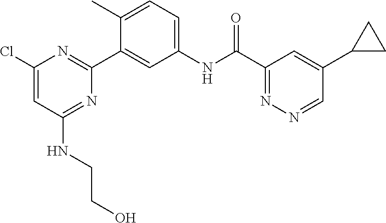 [SMILES: Cc1ccc(NC(=O)c2cc(C3CC3)cnn2)cc1-c1nc(Cl)cc(NCCO)n1] | 33 | 11.04 (s, 1H), 9.27 (d, J = 2.4 Hz, 1H), 8.35 ( s, 1H), 7.93 (d, J = 2.4 Hz, 1H), 7.81-7.87 (m, 2H), 7.27 (d, J = 8.6 Hz, 1H), 6.53 (s, 1H), 4.79 (t, J = 5.4 Hz, 1H), 3.55 (m, 2H), 3.47 (br m, 2H), 2.48 (s, 3H), 2.15 (m, 1H), 1.18-1.27 (m, 2H), 0.96-1.13 (m, 2H). | 425.1 |
| K11 | D5 J1 separation | 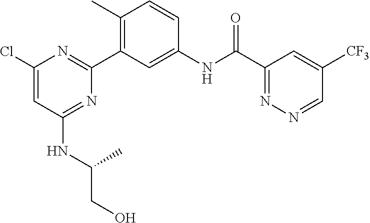 [SMILES: Cc1ccc(NC(=O)c2cc(C(F)(F)F)cnn2)cc1-c1nc(Cl)cc(N[C@H](C)CO)n1] | 29 | 11.35 (s, 1H), 9.99 (s, 1H), 8.53 (s, 1H), 8.08 (s, 1H), 7.89 (m, 1H), 7.32 (d, J = Hz, 1H), 6.58 (s, 1H), 5.75 (s, 1H), 4.90 (br m, 1H), 4.10 (br s, 1H), 3.16-3.25 (m, 2H), 2.21 (s, 3H), 1.16 (d, J = 6.9 Hz, 3H). | 467.2 |
| K12 | D5 J1 separation | 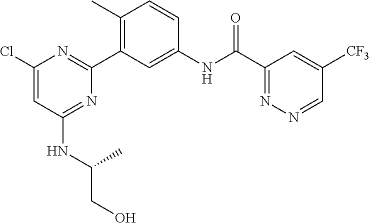 [SMILES: Cc1ccc(NC(=O)c2cc(C(F)(F)F)cnn2)cc1-c1nc(Cl)cc(N[C@H](C)CO)n1] | 40 | 11.35 (s, 1H), 9.99 (s, 1H), 8.62 (s, 1H), 8.40 (d, J = 2.4 Hz, 1H), 8.00 (m, 1H), 7.66 (br m, 1H), 7.34 (d, J = 8.4 Hz, 1H), 6.53 (s, 1H), 4.84 (t, J = 5.5 Hz, 1H), 4.22 (br s, 1H), 3.50 (m, 1H), 3.43 (m, 1H), 1.99 (s, 3H), 1.20 (d, J = 6.9 Hz, 3H). | 467.2 |
| K13 | D6 J1 | 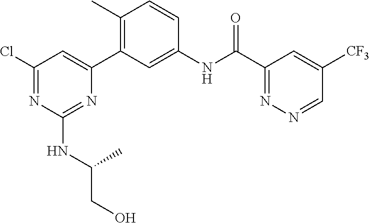 [SMILES: Cc1ccc(NC(=O)c2cc(C(F)(F)F)cnn2)cc1-c1cc(Cl)nc(N[C@H](C)CO)n1] | crude | No Data | 467.2 |
| K14 | 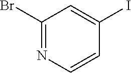 J5 | 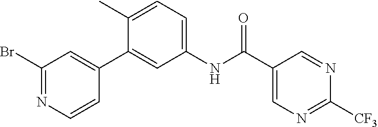 [SMILES: Cc1ccc(NC(=O)c2cnc(C(F)(F)F)nc2)cc1-c1ccnc(Br)c1] | 57 | 10.83 (br s, 1H), 9.49-9.51 (m, 2H), 8.48 (d, J = 5.0 Hz, 1H), 7.75 (dd, J = 1.6, 8.4 Hz, 1H), 7.71 (s, 1H), 7.69 (s, 1H), 7.50 (d, J = 4.8 Hz, 1H), 7.39 (d, J = 8.3 Hz, 1H), 2.26 (s, 3H). | 436.7 |
| K17 | D7 J1 | 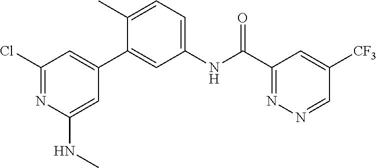 [SMILES: CNc1cc(-c2cc(NC(=O)c3cc(C(F)(F)F)cnn3)ccc2C)cc(Cl)n1] | 34 | 11.29 (s, 1H), 9.96 (d, J = 1.6 Hz, 1H), 8.57 (s, 1H), 7.90 (dd, J = 2.0, 8.4 Hz, 1H), 7.87 (s, 1H), 7.33 (d, J = 8.4 Hz, 1H), 7.04 (q, J = 4.6 Hz, 1H), 6.50 (s, 1H), 6.37 (s, 1H), 2.80 (d, J = 4.8 Hz, 3H), 2.25 (s, 3H). | 422.1 |
| K18 | D8 J7 | 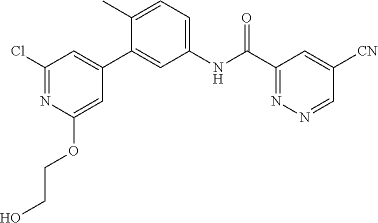 [SMILES: Cc1ccc(NC(=O)c2cc(C#N)cnn2)cc1-c1cc(Cl)nc(OCCO)c1] | 32 | 11.26 (s, 1H), 9.87 (d, J = 2.0 Hz, 1H), 8.78 (d, J = 2.0 Hz, 1H), 7.92 (dd, J = 2.0, 8.4 Hz 1H), 7.88 (d, J = 2.0 Hz, 1H), 7.35 (d, J = 8.4 Hz, 1H), 7.11 (s, 1H), 6.82 (s, 1H), 4.91 (t, J = 5.6 Hz, 1H), 4.31 (t, J = 5.0 Hz, 2H), 3.73 (q, J = 5.2 Hz, 2H), 2.26 (s, 3H). | 410.0 |
| K19 | D3 J7 | 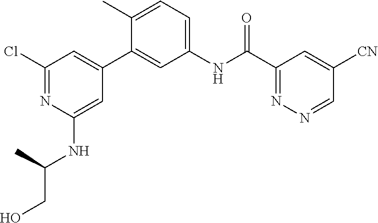 [SMILES: Cc1ccc(NC(=O)c2cc(C#N)cnn2)cc1-c1cc(Cl)nc(N[C@H](C)CO)c1] | 66 | 11.30 (s, 1H), 9.93 (d, J = 2.0 Hz, 1H), 8.84 (d, J = 2.0 Hz, 1H), 7.91 (m, 2H), 7.37 (d, J = 8.4 Hz, 1H), 6.91 (d, J = 7.6 Hz, 1H), 6.53 (s, 1H), 6.47 (s, 1H), 4.80 (t, J = 5.4 Hz, 1H), 3.98 (m, 1H), 3.53 (m, 1H), 3.35 (m, 1H), 2.30 (s, 3H), 1.19 (d, J = 6.4 Hz, 3H). | 423.3 |
| K20 | 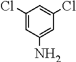 J1 | 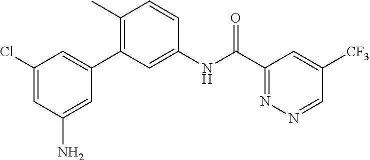 [SMILES: Cc1ccc(NC(=O)c2cc(C(F)(F)F)cnn2)cc1-c1cc(N)cc(Cl)c1] | 90 | No Data | 407.0 |
| K21 | D4 J7 | 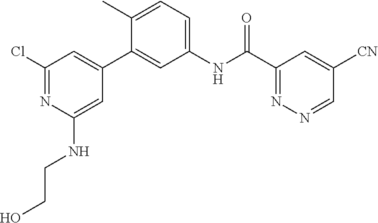 [SMILES: Cc1ccc(NC(=O)c2cc(C#N)cnn2)cc1-c1cc(Cl)nc(NCCO)c1] | 71 | 11.25 (s, 1H), 9.88 (d, J = 2.0 Hz, 1H), 8.80 (d, J = 2.0 Hz, 1H), 7.85- 7.89 (m, 2H), 7.33 (d, J = 8.4 Hz, 1H), 7.08 (t, J = 5.6 Hz, 1H), 6.50 (s, 1H), 6.45 (s, 1H), 4.74 (t, J = 5.4 Hz, 1H), 3.53-3.58 (m, 2H), 3.32 (m, 2H), 2.25 (s, 3H). | 409.3 |

### Preparation of Intermediate K16: N-(3-(2-(2-((tert-butyldimethylsilyl)oxy)ethoxy)-6-chloropyridin-4-yl)-4-methylphenyl)-5-(trifluoromethyl)pyridazine-3-carboxamide

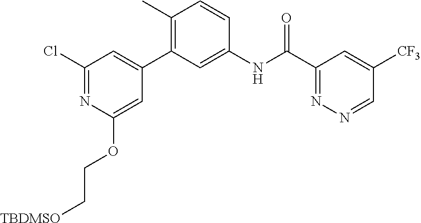 [SMILES: Cc1ccc(NC(=O)c2cc(C(F)(F)F)cnn2)cc1-c1cc(Cl)nc(OCCO[Si](C)(C)C(C)(C)C)c1]

A solution of 2-((tert-butyldimethylsilyl)oxy)ethan-1-ol (K10, 3.5 g, 20 mmol) in 1,4-dioxane (30 mL) was added NaH (1.2 g, 25 mmol). The mixture was stirred at rt for 1 h and then N-(3-(2,6-dichloropyridin-4-yl)-4-methylphenyl)-5-(trifluoromethyl)pyridazine-3-carboxamide (5.0 g, 12 mmol) was added. The reaction mixture was stirred at rt for 1 h, then heated at 70° C. for 1 h. The mixture was cooled rt and then concentrated under reduced pressure. The residue was treated with water, and then extracted with DCM (3×). The combined organic extracts were filtered through a short silica gel column. The filtrate was concentrated to obtain N-(3-(2-(2-((tert-butyldimethylsilyl)oxy)ethoxy)-6-chloropyridin-4-yl)-4-methylphenyl)-5-(trifluoromethyl)pyridazine-3-carboxamide (K16, 5.1 g, 77%) as orange colored liquid. MS (ESI) m/z: 567.2 (M+H⁺).

### Preparation of Example 1: N-(3-(2′-cyano-[2,4′-bipyridin]-4-yl)-4-methylphenyl)-5-(trifluoromethyl)pyridazine-3-carboxamide

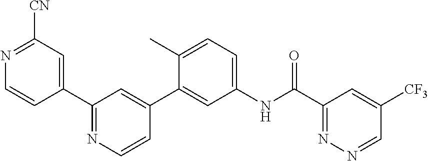 [SMILES: Cc1ccc(NC(=O)c2cc(C(F)(F)F)cnn2)cc1-c1ccnc(-c2ccnc(C#N)c2)c1]

A solution of 4-(4,4,5,5-tetramethyl-1,3,2-dioxaborolan-2-yl)picolinonitrile (0.53 g, 2.3 mmol), and N-(3-(2-chloropyridin-4-yl)-4-methylphenyl)-5-(trifluoromethyl)pyridazine-3-carboxamide (K8, 0.9 g, 2.3 mmol) in a mixture of 1,4-dioxane (5 mL) and water (0.6 mL) was treated with K₂CO₃ (0.79 g, 5.7 mmol). The mixture was purged with Ar for 5 min, then PdCl₂(dppf)·DCM adduct (0.19 g, 0.23 mmol) was added. The reaction mixture was purged with Ar again for an additional 5 min. The reaction was then sealed and heated to 90° C. for 2 h. The reaction mixture was filtered through a pad of celite and washes with EtOAc (200 mL), and 10% EtOAc/MeOH (200 mL). The filtrate was concentrated under reduced pressure. The crude was purified by silica gel column chromatography (0 to 100% EtOAc/hexanes) to obtain N-(3-(2′-cyano-[2,4′-bipyridin]-4-yl)-4-methylphenyl)-5-(trifluoromethyl)pyridazine-3-carboxamide (1, 1.0 g, 95%) as a brown solid. MS m/z: 393.0 (M+H⁺).

The following compounds are prepared essentially by method of preparation Example 1.

| | | | | | MS |
|---|---|---|---|---|---|
| | | | | ¹H NMR (400 | (m/z: |
| Ex | | | Yield | MHz, DMSO-d₆): | M + |
| No | SM | Product | (%) | □ | H⁺) |
| 4 | A1 K15 | 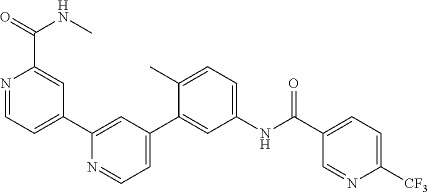 [SMILES: CNC(=O)c1cc(-c2cc(-c3cc(NC(=O)c4ccc(C(F)(F)F)nc4)ccc3C)ccn2)ccn1] | 16 | 10.7 (s, 1H), 9.26 (s, 1H), 8.85 (m, 2H), 8.76 (m, 2H), 8.59 (br s, 1H), 8.30 (m, 1H), 8.19 (s, 1H), 8.11 (d, J = 8.0 Hz, 1H), 7.79 (m, 2H), 7.55 (m, 1H), 7.40 (d, J = 8.4 Hz, 1H), 2.86 (d, J = 4.8 Hz, 3H), 2.30 (s, 3H). | 491.9 |
| 5 | A2 K15 | 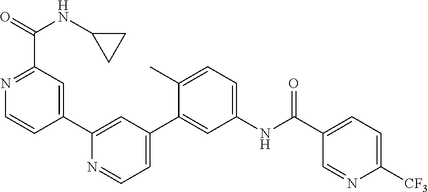 [SMILES: Cc1ccc(NC(=O)c2ccc(C(F)(F)F)nc2)cc1-c1ccnc(-c2ccnc(C(=O)NC3CC3)c2)c1] | 48 | 10.7 (s, 1H), 9.25 (s, 1H), 8.86 (d, J = 4.8 Hz, 1H), 8.81 (q, J = 4.8 Hz, 1H), 8.77 (s, 1H), 8.73 (d, J = 5.2 Hz, 1H), 8.57 (d, J = 9.2 Hz, 1H), 8.33 (dd, J = 1.6, 5.2 Hz, 1H), 8.20 (s, 1H), 8.11 (d, J = 8.4 Hz, 1H), 7.79 (m, 2H), 7.56 (d, J = 4.0 Hz, 1H), 7.40 (d, J = 8.4 Hz, 1H), 2.91-2.97 | 518.2 |
| | | | | (m, 1H), 2.30 (s, 3H), | |
| | | | | 0.71 (br m, 4H). | |
| 8 | A1 K5 | 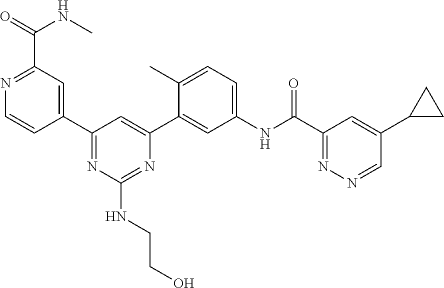 [SMILES: CNC(=O)c1cc(-c2cc(-c3cc(NC(=O)c4cc(C5CC5)cnn4)ccc3C)nc(NCCO)n2)ccn1] | 41 | 11.08 (s, 1H), 9.27 (d, J = 2.2 Hz, 1H), 8.87 (q, J = 4.7 Hz, 1H), 8.79 (d, J = 5.0 Hz, 1H), 8.73 (br s, 1H), 8.29 (br s, 1H), 8.14 (d, J = 2.0 Hz, 1H), 7.96 (br d, J = 8.0 Hz, 1H), 7.94 (d, J = 2.0 Hz, 1H), 7.535 (br m, 1H), 7.45 (s, 1H), 7.34 (d, J = 8.6 Hz, 1H), 4.72 (br m, 1H), 3.55-3.65 (br m, 2H), 3.40-3.555 (br m, 2H), 2.86 (d, J = 4.8 Hz, 3H), 2.43 (s, 3H), 2.15 (m, 1H), 1.16-1.27 (m, 2H), 1.00-1.10 (m, 2H). | 525.2 |
| 19 | A1 K2 | 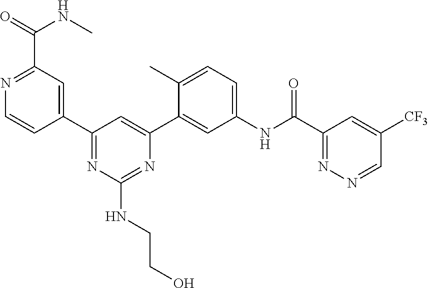 [SMILES: CNC(=O)c1cc(-c2cc(-c3cc(NC(=O)c4cc(C(F)(F)F)cnn4)ccc3C)nc(NCCO)n2)ccn1] | 23 | 11.34 (br s, 1H), 9.96 (d, J = 1.8 Hz, 1H), 8.86 (br q, J = 5.0 Hz, 1H), 8.79 (d, J = 5.2 Hz, 1H), 8.73 (br s, 1H), 8.59 (s, 1H), 8.29 (br m, 1H), 8.15 (d, J = 2.0 Hz, 1H), 7.99 (br d, J = 5.6 Hz, 1H), 7.51 (br s, 1H), 7.45 (s, 1H), 7.38 (d, J = 8.4 Hz, 1H), 4.72 (br m, 1H), 3.59 (br m, 2H), 3.49 (br m, 2H), 2.86 (d, J = 5.0 Hz, 3H), 2.49 (s, 3H). | 553.1 |
| 22 | A1 K3 | 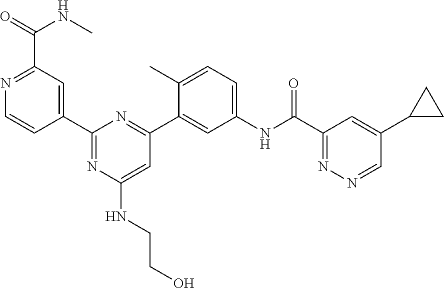 [SMILES: CNC(=O)c1cc(-c2nc(NCCO)cc(-c3cc(NC(=O)c4cc(C5CC5)cnn4)ccc3C)n2)ccn1] | 28 | 11.09 (s, 1H), 9.27 (d, J = 2.4 Hz, 1H), 8.89 (s, 1H), 8.83 (q, J = 4.8 Hz, 1H), 8.77 (q, J = 5.0 Hz, 1H), 8.42 ( d, J = 4.6 Hz, 1H), 8.12 (s, 1H), 7.89- 7.97 (m, 2H), 7.80 (br s, 1H), 7.35 (d, J = 8.4 Hz, 1H), 6.71 (s, 1H), 4.85 (br m, 1H), 3.55-3.72 (br m, 4H), 2.85 (d, J = 4.8 Hz, 3H), 2.41 (s, 3H), 2.14 (m, 1H), 1.16-1.27 (m, 2H), 0.99-1.10 (m, 2H). | 525.2 |
| 23 | A1 K4 | 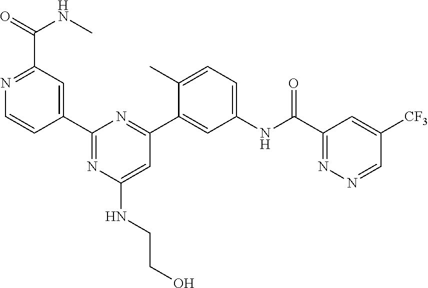 [SMILES: CNC(=O)c1cc(-c2nc(NCCO)cc(-c3cc(NC(=O)c4cc(C(F)(F)F)cnn4)ccc3C)n2)ccn1] | 24 | 11.37 (s, 1H), 9.97 (d, J = 1.6 Hz, 1H), 8.91 (s, 1H), 8.85 (q, J = 4.8 Hz, 1H), 8.77 (q, J = 4.8 Hz, 1H), 8.58 (s, 1H), 8.42 (br m, 1H), 8.14 (s, 1H), 7.94 (br m, 1H), 7.83 (br m, 1H), 7.38 (d, J = 8.1 Hz, 1H), 6.72 (s, 1H), 4.86 (br m, 1H), 3.60-3.68 (m, 4H), 2.85 (d, J = 4.8 Hz, 3H), 2.42 (s, 3H). | 553.0 |
| 24 | B6 J2 | 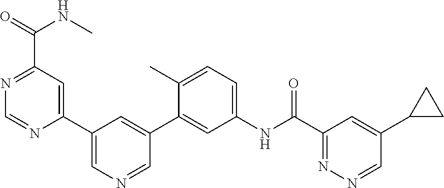 [SMILES: CNC(=O)c1cc(-c2cncc(-c3cc(NC(=O)c4cc(C5CC5)cnn4)ccc3C)c2)ncn1] | 51 | 11.09 (s, 1H), 9.45 (d, J = 7.2 Hz, 2H), 9.28 (d, J = 2.0 Hz, 1H), 9.09 (q, J = 4.4 Hz, 1H), 8.83 (d, J = 2.0 Hz, 1H), 8.68 (s, 1H), 8.65 (s, 1H), 7.94-7.99 (m, 3H), 7.40 (d, J = 8.4 Hz, 1H), 2.88 (d, J = 4.8 Hz, 3H), 2.31 (s, 3H), 2.16 (m, 1H), 1.21-1.26 (m, 2H), 1.03-1.07 (m, 2H). | 466.1 |
| 25 | A1 H13 | 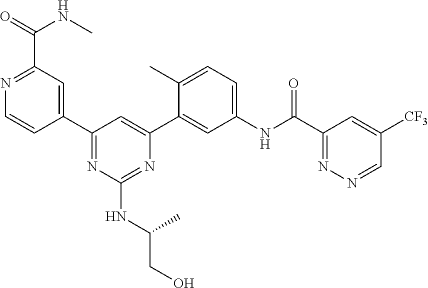 [SMILES: CNC(=O)c1cc(-c2cc(-c3cc(NC(=O)c4cc(C(F)(F)F)cnn4)ccc3C)nc(N[C@H](C)CO)n2)ccn1] | 15 | 11.33 (s, 1H), 9.96 (s, 1H), 8.81 (m, 3H), 8.59 (s, 1H), 8.29 (s, 1H), 8.15 (d, J = 2.3 Hz, 1H), 7.99 (d, J = 8.3 Hz, 1H), 7.44 (s, 1H), 7.37 (d, J = 8.4 Hz, 1H), 7.27 (s, 1H), 4.70 (s, 1H), 4.14 (s, 1H), 3.48 (m, 2H), 2.86 (d, J = 4.8 Hz, 3H), 2.45 (s, 3H), 1.21 (d, J = 6.6 Hz, 3H). | 567.2 |
| 28 | A1 K11 | 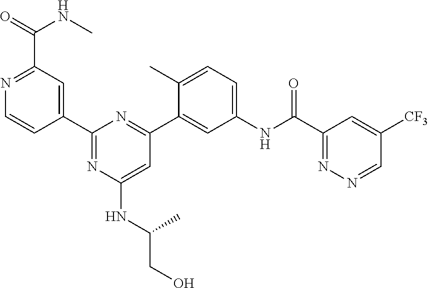 [SMILES: CNC(=O)c1cc(-c2nc(N[C@H](C)CO)cc(-c3cc(NC(=O)c4cc(C(F)(F)F)cnn4)ccc3C)n2)ccn1] | 53 | 11.33 (s, 1H), 9.94 (d, J = 2.2 Hz, 1H), 8.87 (s, 1H), 8.82 (q, J = 5.0 Hz, 1H), 8.75 (d, J = 5.0 Hz, 1H), 8.56 (s, 1H), 8.37- 8.42 (m, 1H), 8.13 (m, 1H), 7.92 (d, J = 8.4 Hz, 1H), 7.58 (s, 1H), 7.36 (d, J = 8.3 Hz, 1H), 6.68 (s, 1H), 4.83 (t, J = 5.4 Hz, 1H), 4.36 (s, 1H), 3.51(m, 2H), 2.83 (d, J = 4.8 Hz, 3H), 2.41 (s, 3H), 1.22 (d, J = 6.6 Hz, 3H). | 567.2 |
| 33 | A1 K6 | 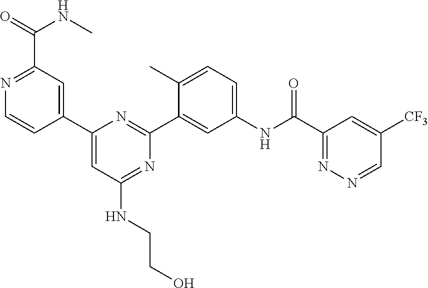 [SMILES: CNC(=O)c1cc(-c2cc(NCCO)nc(-c3cc(NC(=O)c4cc(C(F)(F)F)cnn4)ccc3C)n2)ccn1] | 41 | 11.37 (s, 1H), 9.96 (s, 1H), 8.89 (q, J = 4.8 Hz, 1H), 8.83 (d, J = 5.2 Hz, 1H), 8.58 (br m, 2H), 8.42 (s, 1H), 8.16 (br m, 1H), 7.97 (br d, J = 7.9 Hz, 1H), 7.37 (d, J = 8.3 Hz, 1H), 7.21 (s, 1H), 3.59-3.45 (br m, 2H), 3.50-3.59 (br s, 2H), 2.85 (d, J = 4.8 Hz, 3H), 2.54 (s, 3H), NH and OH are under solvents. | 553.2 |
| 34 | A1 K9 | 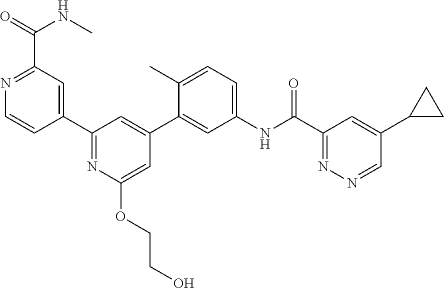 [SMILES: CNC(=O)c1cc(-c2cc(-c3cc(NC(=O)c4cc(C5CC5)cnn4)ccc3C)cc(OCCO)n2)ccn1] | 34 | 11.06 (s, 1H), 9.27 (d, J = 2.1 Hz, 1H), 8.84 (q, J = 4.8 Hz, 1H), 8.69- 8.76 (m, 2H), 8.31 (dd, J = 1.7, 5.1 Hz, 1H), 7.89-7.99 (m, 3H), 7.80 (s, 1H), 7.37 (d, J = 8.2 Hz, 1H), 6.94 (d, J = 1.2 Hz, 1H), 4.91 (s, 1H), 4.50 (t, J = 5.1 Hz, 2H), 3.83 (t, J = 5.2 Hz, 2H), 2.86 (d, J = 4.8 Hz, 3H), 2.31 (s, 3H), 2.15 (m, 1H), 1.18-1.27 (m, 2H), 1.08-1.00 (m, 2H). | 525.2 |
| 35 | A1 K16 TBAF | 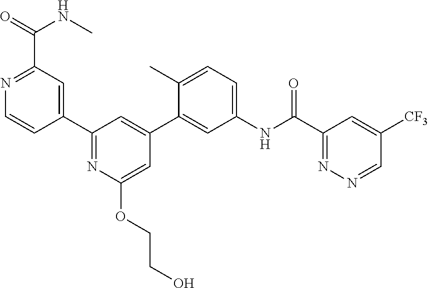 [SMILES: CNC(=O)c1cc(-c2cc(-c3cc(NC(=O)c4cc(C(F)(F)F)cnn4)ccc3C)cc(OCCO)n2)ccn1] | 58 | 11.32 (s, 1H), 9.96 (d, J = 2.2 Hz, 1H), 8.84 (q, J = 4.8 Hz, 1H), 8.68- 8.78 (m, 2H), 8.57 (m, 1H), 8.31 (dd, J = 1.8, 5.2 Hz, 1H), 7.97 (d, J = 7.3 Hz, 2H), 7.81 (d, J = 1.2 Hz, 1H), 7.39 (d, J = 8.3 Hz, 1H), 6.95 (d, J = 1.2 Hz, 1H), 4.92 (t, J = 5.5 Hz, 1H), 4.51 (t, J = 5.2 Hz, 2H), 3.83 (m, 2H), 2.86 (m, 3H), 2.32 (s, 3H). | 553.2 |
| 39 | A1 K12 | 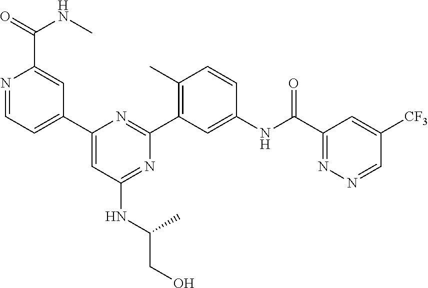 [SMILES: CNC(=O)c1cc(-c2cc(N[C@H](C)CO)nc(-c3cc(NC(=O)c4cc(C(F)(F)F)cnn4)ccc3C)n2)ccn1] | 31 | 11.31 (s, 1H), 9.94 (d, J = 2.3 Hz, 1H), 8.84 (q, J = 4.8 Hz, 1H), 8.78 (d, J = 5.1 Hz, 1H), 8.61 (br s, 1H), 8.59 (s, 1H), 8.43 (s, 1H), 8.19 (br s, 1H), 7.94 (br d, J = 8.3 Hz, 1H), 7.54 (br s, 1H), 7.33 (d, J = 8.3 Hz, 1H), 7.17 (s, 1H), 4.81 (t, J = 5.6 Hz, 1H), 4.25 (br s, 1H), 3.54 (m, 1H), 3.45 (m, 1H), 2.84 (d, J = 4.8 Hz, 3H), 2.57 (s, 3H), 1.20 (d, J = 6.6 Hz, 3H). | 567.2 |
| 40 | A1 K7 | 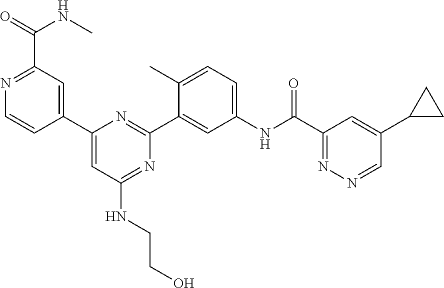 [SMILES: CNC(=O)c1cc(-c2cc(NCCO)nc(-c3cc(NC(=O)c4cc(C5CC5)cnn4)ccc3C)n2)ccn1] | 3 | 11.12 (s, 1H), 9.33 (d, J = 2.0 Hz, 1H), 8.93 (q, J = 4.8 Hz, 1H), 8.86 (d, J = 5.2 Hz, 1H), 8.66 (br s, 1H), 8.48 (s, 1H), 8.27 (br s, 1H), 7.96- 8.01 (m, 2H), 7.81 (br s, 1H), 7.37 (d, J = 8.4 Hz, 1H), 7.25 (s, 1H), 4.88 (t, J = 5.2 Hz, 1H), 3.64-3.70 (m, 2H), 3.58-3.63 (m, 2H), 2.92 (d, J = 4.8 Hz, 3H), 2.69 (s, 3H), 2.15 (m, 1H), 1.22-1.35 (m, 2H), 1.06-1.16 (m, 2H). | 525.1 |
| 41 | A1 K14 | 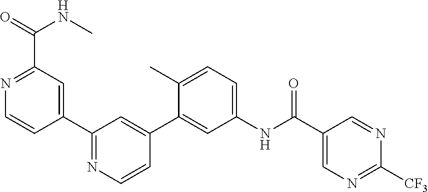 [SMILES: CNC(=O)c1cc(-c2cc(-c3cc(NC(=O)c4cnc(C(F)(F)F)nc4)ccc3C)ccn2)ccn1] | 23 | 10.86 (s, 1H), 9.51 (s, 2H), 8.87 (m, 2H), 8.73-8.81 (m, 2H), 8.35 (br d, J = 3.6 Hz, 1H), 8.20 (s, 1H), 7.73-7.84 (m, 2H), 7.57 (br d, J = 4.0 Hz, 1H), 7.44 (d, J = 8.4 Hz, 1H), 2.87 (d, J = 4.8 Hz, 3H), 2.32 (s, 3H). | 493.1 |
| 46 | B10 J7 | 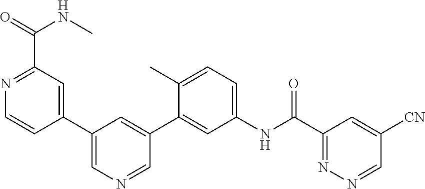 [SMILES: CNC(=O)c1cc(-c2cncc(-c3cc(NC(=O)c4cc(C#N)cnn4)ccc3C)c2)ccn1] | 16 | 11.27 (s, 1H), 9.87 (d, J = 1.6 Hz, 1H), 9.08 (d, J = 2.4 Hz, 1H), 8.86 (q, J = 4.8 Hz, 1H), 8.80 (d, J = 2.0 Hz, 1H), 8.73-8.76 (m, 2H), 8.40 (d, J = 1.6 Hz, 1H), 8.29 (t, J = 2.4 Hz, 1H), 8.08 (dd, J = 2.0, 5.2 Hz, 1H), 7.94-7.98 (m, 2H), 7.40 (d, J = 9.2 Hz, 1H), 2.85 (d, J = 4.8 Hz, 3H), 2.31 (s, 3H). | 450.0 |
| 47 | A1 K17 | 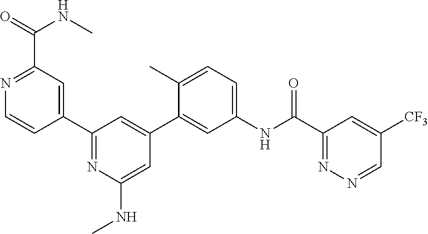 [SMILES: CNC(=O)c1cc(-c2cc(-c3cc(NC(=O)c4cc(C(F)(F)F)cnn4)ccc3C)cc(NC)n2)ccn1] | 28 | 11.32 (s, 1H), 9.97 (d, J = 1.8 Hz, 1H), 8.82 (q, J = 4.8 Hz, 1H), 8.70 (m, 2H), 8.58 (d, J = 1.4 Hz, 1H), 8.24 (d, J = 5.2 Hz, 1H), 7.91-7.96 (m, 2H), 7.38 (d, J = 9.0 Hz, 1H), 7.28 (s, 1H), 6.92 (d, J = 5.0 Hz, 1H), 6.56 (s, 1H), 2.95 (d, J = 4.8 Hz, 3H), 2.86 (d, J = 4.8 Hz, 3H), 2.31 (s, 3H). | 522.1 |
| 48 | B11 J1 cata CXium (R) A-Pd- G2 | 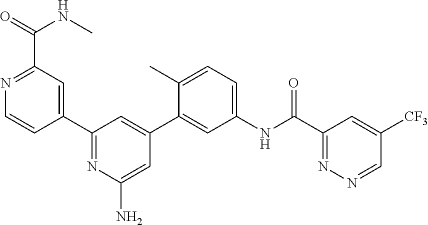 [SMILES: CNC(=O)c1cc(-c2cc(-c3cc(NC(=O)c4cc(C(F)(F)F)cnn4)ccc3C)cc(N)n2)ccn1] | 56 | 11.32 (s, 1H), 9.97 (s, 1H), 8.82 (q, J = 4.8 Hz, 1H), 8.65-8.74 (m, 2H), 8.59 (s, 1H), 8.19 (d, J = 4.0 Hz, 1H), 7.88-8.01 (m, 2H), 7.38 (d, J = 8.0 Hz, 1H), 7.29 (s, 1H), 6.57 (s, 1H), 6.40 (s, 2H), 2.86 (d, J = 4.6 Hz, 3H), 2.27 (s, 3H). | 508.0 |
| 49 | A1 K18 | 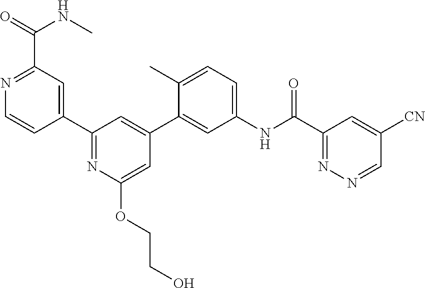 [SMILES: CNC(=O)c1cc(-c2cc(-c3cc(NC(=O)c4cc(C#N)cnn4)ccc3C)cc(OCCO)n2)ccn1] | 14 | 11.27 (s, 1H), 9.87 (d, J = 1.6 Hz, 1H), 8.84 (q, J = 4.4 Hz, 1H), 8.80 (d, J = 1.6 Hz, 1H), 8.74 (d, J = 5.2 Hz, 1H), 8.71 (s, 1H), 8.31 (d, J = 4.4 Hz, 1H), 7.93-7.98 (m, 2H), 7.81 (s, 1H), 7.39 (d, J = 8.4 Hz, 1H), 6.95 (s, 1H), 4.91 (t, J = 5.6 Hz, 1H), 4.50 (t, J = 4.8 Hz, 2H), 3.83 (q, J = 5.2 Hz, 2H), 2.85 (d, J = 4.4 Hz, 3H), 2.31 (s, 3H). | 510.2 |
| 50 | A1 K19 | 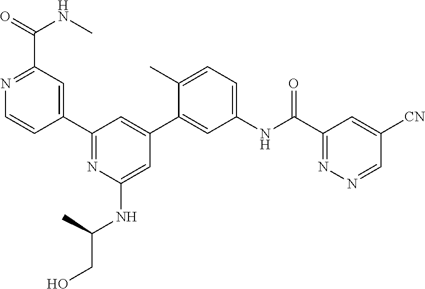 [SMILES: CNC(=O)c1cc(-c2cc(-c3cc(NC(=O)c4cc(C#N)cnn4)ccc3C)cc(N[C@H](C)CO)n2)ccn1] | 19 | 11.26 (s, 1H), 9.88 (d, J = 2.0 Hz, 1H), 8.78-8.85 (m, 2H), 8.69 (d, J = 5.2 Hz, 1H), 8.65 (s, 1H), 8.22 (dd, J = 1.6, 5.2 Hz, 1H), 7.88-7.93 (m, 2H), 7.37 (d, J = 8.0 Hz, 1H), 7.24 (s, 1H), 6.70 (d, J = 8.0 Hz, 1H), 6.61 (s, 1H), 4.77 (t, J = 5.6 Hz, 1H), 4.16 (m, 1H), 3.57 (m, 1H), 3.46 (m, 1H), 2.85 (d, J = 4.8 Hz, 3H), 2.31 (s, 3H), 1.23 (d, J = 6.8 Hz, 3H). | 523.2 |
| 51 | A1 K20 | 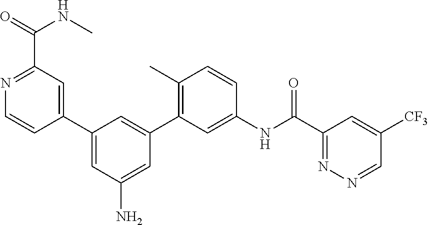 [SMILES: CNC(=O)c1cc(-c2cc(N)cc(-c3cc(NC(=O)c4cc(C(F)(F)F)cnn4)ccc3C)c2)ccn1] | 65 | 11.24 (s, 1H), 9.93 (d, J = 2.2 Hz, 1H), 8.81 (q, J = 4.8 Hz, 1H), 8.63 (d, J = 5.1 Hz, 1H), 8.55 (d, J = 1.8 Hz, 1H), 8.23 (d, J = 1.8 Hz, 1H), 7.89 (d, J = 2.2 Hz, 1H), 7.80-7.87 (m, 2H), 7.30 (d, J = 8.4 Hz, 1H), 7.03 (br s, 1H), 6.89 (br s, 1H), 6.68 (br s, 1H), 5.47 (s, 2H), 2.83 (d, J = 4.8 Hz, 3H), 2.27 (s, 3H). | 507.2 |
| 53 137 54 | A1 K21 | 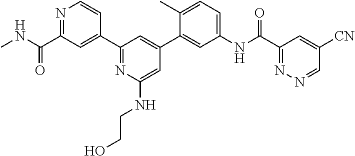 [SMILES: CNC(=O)c1cc(-c2cc(-c3cc(NC(=O)c4cc(C#N)cnn4)ccc3C)cc(NCCO)n2)ccn1] | 21 | 11.30 (s, 1H), 9.91 (s, 1H), 8.86 (q, J = 4.8 Hz, 1H), 8.84 (d, J = 2.0 Hz, 1H), 8.73 (d, J = 5.2 Hz, 1H), 8.69 (br s, 1H), 8.26 (dd, J = 2.0, 5.2 Hz, 1H), 7.92-7.97 (m, 2H), 7.40 (d, J = 8.4 Hz, 1H), 7.29 (s, 1H), 6.99 (t, J = 5.2 Hz, 1H), 6.67 (s, 1H), 4.84 (t, J = 5.4 Hz, 1H), 3.67-3.72 (m, 2H), 3.53-3.58 (m, 2H), 2.89 (d, J = 4.8 Hz, 3H), 2.34 | 509.2 |
| | | | | (s, 3H). | |

### Preparation of Example 2: N,6-dimethyl-4-(2-methyl-5-(5-(trifluoromethyl)pyridazine-3-carboxamido)phenyl)-[2,4′-bipyridine]-2′-carboxamide

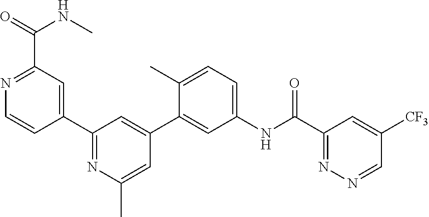 [SMILES: CNC(=O)c1cc(-c2cc(-c3cc(NC(=O)c4cc(C(F)(F)F)cnn4)ccc3C)cc(C)n2)ccn1]

A mixture of 5-(trifluoromethyl)pyridazine-3-carboxylic acid (0.14 g, 0.75 mmol), 4-(5-amino-2-methylphenyl)-N, 6-dimethyl-[2,4′-bipyridine]-2′-carboxamide (F3, 0.25 g, 0.75 mmol), pyridine (0.18 mL) and EDC (0.23 g, 1.50 mmol) in DMF (4 mL) was stirred at rt for 1 h. The reaction mixture was poured into water and the resulting solid was filtered and washed with water. The solid was purified by silica gel column chromatography (0 to 10000 EtOAc/hexanes) to obtain N,6-dimethyl-4-(2-methyl-5-(5-(trifluoromethyl)pyridazine-3-carboxamido)phenyl)-[2,4′-bipyridine]-2′-carboxamide (0.26 g, 670%) as a white solid. ¹H NMR (400 Uz, DMSO-d₆): δ 11.32 (s, 1H), 9.95 (d, J=2.2 Hz, 1H), 8.83 (q, J=4.8 Hz, 1H), 8.75 (d, J=1.8 Hz, 1H), 8.73 (d, J=5.1 Hz, 1H), 8.57 (d, J=2.3 Hz, 1H), 8.30 (dd, J=1.8, 5.2 Hz, 1H), 7.93-8.01 (i, 3H), 7.43 (s, 1H), 7.38 (m, 1H), 2.85 (d, J=4.8 Hz, 3H), 2.66 (s, 3H), 2.31 (s, 3H); MS m/z: 507.2 (M+H2).

The following compounds are prepared essentially by method of preparation Example 2.

| Ex | | | Yield |
|---|---|---|---|
| No | SM | Product | (%) |
| 6 | F10 I4 EDC | 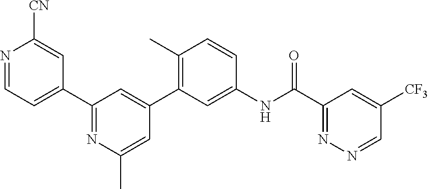 [SMILES: Cc1cc(-c2cc(NC(=O)c3cc(C(F)(F)F)cnn3)ccc2C)cc(-c2ccnc(C#N)c2)n1] | 46 |
| 9 | F1 I4 HATU | 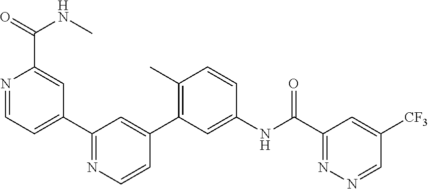 [SMILES: CNC(=O)c1cc(-c2cc(-c3cc(NC(=O)c4cc(C(F)(F)F)cnn4)ccc3C)ccn2)ccn1] | 75 |
| 10 | F4 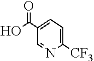 T3P | 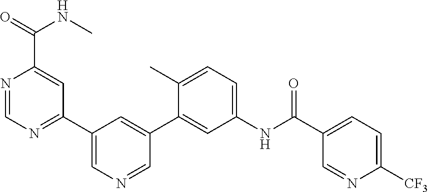 [SMILES: CNC(=O)c1cc(-c2cncc(-c3cc(NC(=O)c4ccc(C(F)(F)F)nc4)ccc3C)c2)ncn1] | 31 |
| 11 | F6 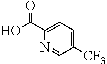 T3P | 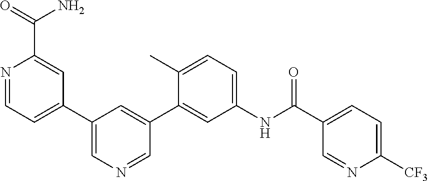 [SMILES: Cc1ccc(NC(=O)c2ccc(C(F)(F)F)nc2)cc1-c1cncc(-c2ccnc(C(N)=O)c2)c1] | 5 |
| 12 | F1 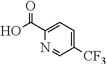 EDC | 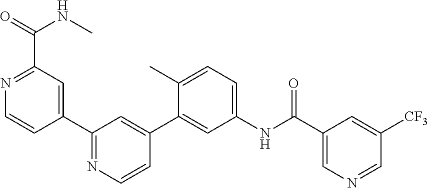 [SMILES: CNC(=O)c1cc(-c2cc(-c3cc(NC(=O)c4cncc(C(F)(F)F)c4)ccc3C)ccn2)ccn1] | 87 |
| 13 | F3 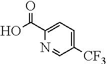 EDC | 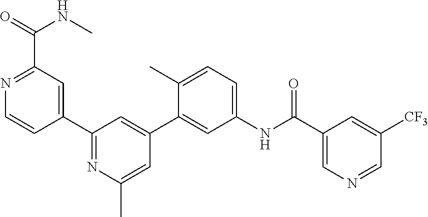 [SMILES: CNC(=O)c1cc(-c2cc(-c3cc(NC(=O)c4cncc(C(F)(F)F)c4)ccc3C)cc(C)n2)ccn1] | 51 |
| 14 | F1 I3 HATU | 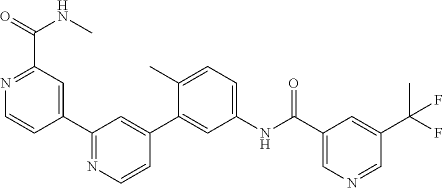 [SMILES: CNC(=O)c1cc(-c2cc(-c3cc(NC(=O)c4cncc(C(C)(F)F)c4)ccc3C)ccn2)ccn1] | 48 |
| 15 | F3 I3 EDC | 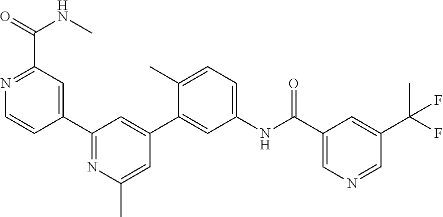 [SMILES: CNC(=O)c1cc(-c2cc(-c3cc(NC(=O)c4cncc(C(C)(F)F)c4)ccc3C)cc(C)n2)ccn1] | 58 |
| 16 | F2 I4 HATU | 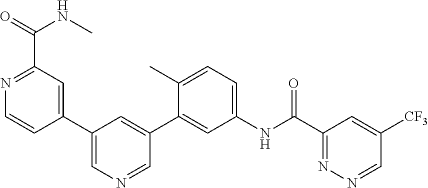 [SMILES: CNC(=O)c1cc(-c2cncc(-c3cc(NC(=O)c4cc(C(F)(F)F)cnn4)ccc3C)c2)ccn1] | 68 |
| 17 | F4 I4 HATU | 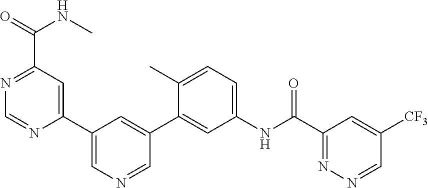 [SMILES: CNC(=O)c1cc(-c2cncc(-c3cc(NC(=O)c4cc(C(F)(F)F)cnn4)ccc3C)c2)ncn1] | 47 |
| 18 | F5 I4 HATU | 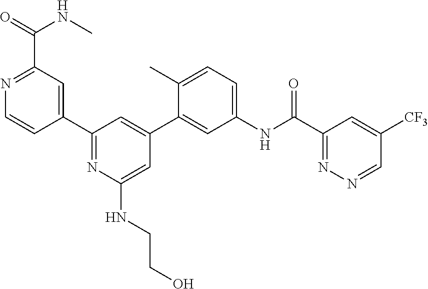 [SMILES: CNC(=O)c1cc(-c2cc(-c3cc(NC(=O)c4cc(C(F)(F)F)cnn4)ccc3C)cc(NCCO)n2)ccn1] | 51 |
| 20 | F1 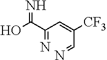 | 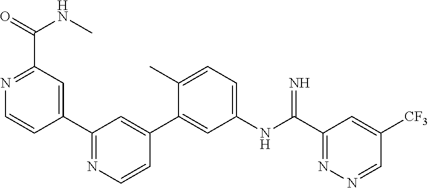 [SMILES: CNC(=O)c1cc(-c2cc(-c3cc(NC(=N)c4cc(C(F)(F)F)cnn4)ccc3C)ccn2)ccn1] | 31 |
| 21 | F1 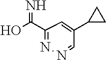 | 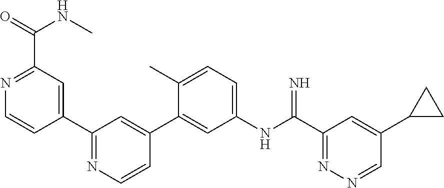 [SMILES: CNC(=O)c1cc(-c2cc(-c3cc(NC(=N)c4cc(C5CC5)cnn4)ccc3C)ccn2)ccn1] | 10 |
| 26 | F1 I1 HATU | 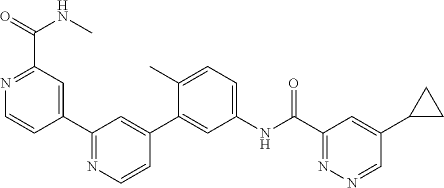 [SMILES: CNC(=O)c1cc(-c2cc(-c3cc(NC(=O)c4cc(C5CC5)cnn4)ccc3C)ccn2)ccn1] | 62 |
| 27 | F5 I1 HATU | 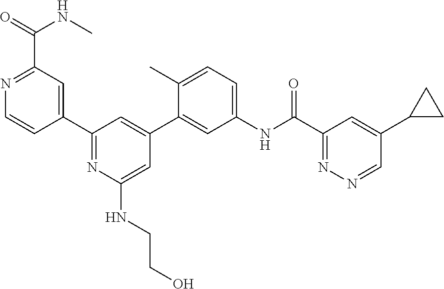 [SMILES: CNC(=O)c1cc(-c2cc(-c3cc(NC(=O)c4cc(C5CC5)cnn4)ccc3C)cc(NCCO)n2)ccn1] | 26 |
| 29 | F7 I4 HATU | 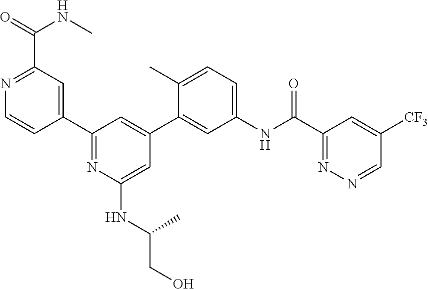 [SMILES: CNC(=O)c1cc(-c2cc(-c3cc(NC(=O)c4cc(C(F)(F)F)cnn4)ccc3C)cc(N[C@H](C)CO)n2)ccn1] | 13 |
| 30 | F1 I2 HATU | 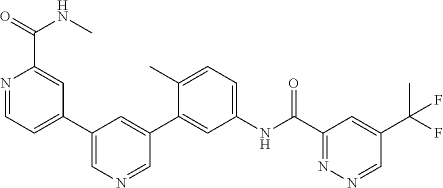 [SMILES: CNC(=O)c1cc(-c2cncc(-c3cc(NC(=O)c4cc(C(C)(F)F)cnn4)ccc3C)c2)ccn1] | 76 |
| 31 | F1 I2 HATU | 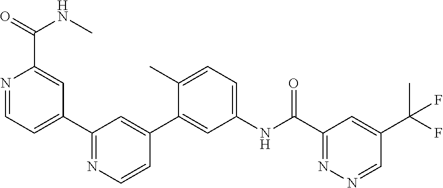 [SMILES: CNC(=O)c1cc(-c2cc(-c3cc(NC(=O)c4cc(C(C)(F)F)cnn4)ccc3C)ccn2)ccn1] | 96 |
| 32 | F7 I1 HATU | 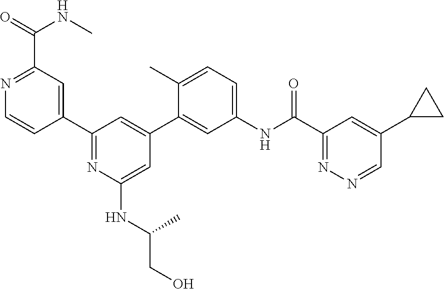 [SMILES: CNC(=O)c1cc(-c2cc(-c3cc(NC(=O)c4cc(C5CC5)cnn4)ccc3C)cc(N[C@H](C)CO)n2)ccn1] | 19 |
| 36 | F1 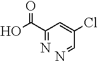 EDC | 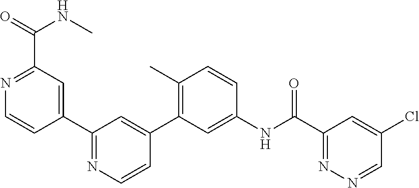 [SMILES: CNC(=O)c1cc(-c2cc(-c3cc(NC(=O)c4cc(Cl)cnn4)ccc3C)ccn2)ccn1] | 21 |
| 37 | F11 I4 HATU | 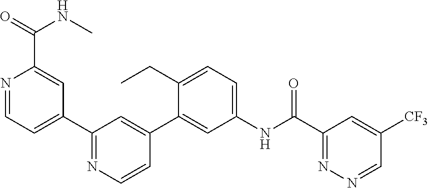 [SMILES: CCc1ccc(NC(=O)c2cc(C(F)(F)F)cnn2)cc1-c1ccnc(-c2ccnc(C(=O)NC)c2)c1] | 30 |
| 38 | F7 I2 HATU | 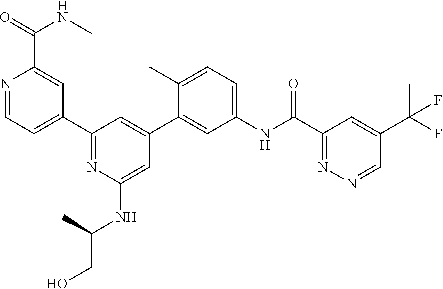 [SMILES: CNC(=O)c1cc(-c2cc(-c3cc(NC(=O)c4cc(C(C)(F)F)cnn4)ccc3C)cc(N[C@H](C)CO)n2)ccn1] | 59 |
| 42 | F5 I2 T3P | 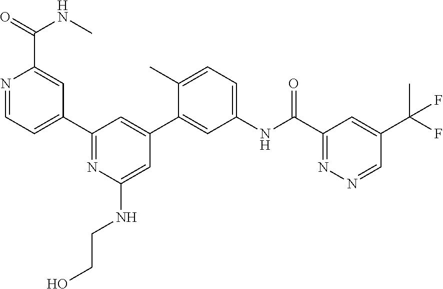 [SMILES: CNC(=O)c1cc(-c2cc(-c3cc(NC(=O)c4cc(C(C)(F)F)cnn4)ccc3C)cc(NCCO)n2)ccn1] | 35 |
| 43 | F12 I2 HATU | 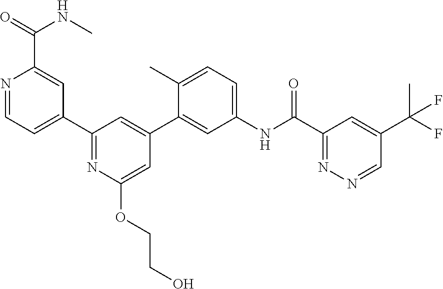 [SMILES: CNC(=O)c1cc(-c2cc(-c3cc(NC(=O)c4cc(C(C)(F)F)cnn4)ccc3C)cc(OCCO)n2)ccn1] | 13 |
| 44 | F7 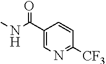 HATU | 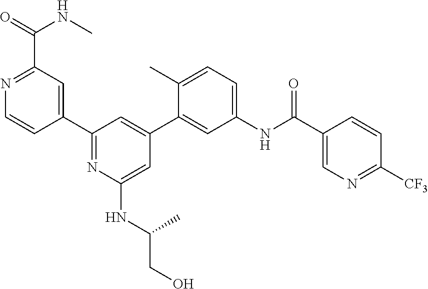 [SMILES: CNC(=O)c1cc(-c2cc(-c3cc(NC(=O)c4ccc(C(F)(F)F)nc4)ccc3C)cc(N[C@H](C)CO)n2)ccn1] | 32 |
| 45 | F7 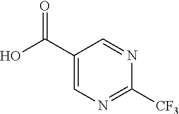 HATU | 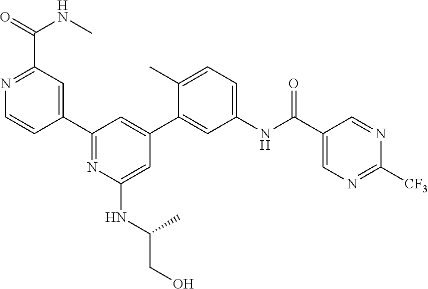 [SMILES: CNC(=O)c1cc(-c2cc(-c3cc(NC(=O)c4cnc(C(F)(F)F)nc4)ccc3C)cc(N[C@H](C)CO)n2)ccn1] | 64 |

| | | MS |
|---|---|---|
| | | (m/z: |
| Ex | ¹H NMR (400 MHz, | M + |
| No | DMSO-d₆): □ | H⁺) |
| 6 | No Data | 475.2 |
| 9 | 11.36 (s, 1H), 9.96 (d, J = 2.2 Hz, | 493.0 |
| | 1H), 8.82-8.88 (m, 2H), 8.78 (d, J = | |
| | 0.8 Hz, 1H), 8.76 (q, J = 5.1 | |
| | Hz, 1H), 8.58 (d, J = 0.8 Hz, 1H), | |
| | 8.34 (dd, J = 1.8, 5.2 Hz, 1H), | |
| | 8.21 (s, 1H), 7.97-8.01 (m, 2H), | |
| | 7.58 (dd, J = 1.5, 5.0 Hz, 1H), | |
| | 7.41 (d, J = 8.9 Hz, 1H), 2.86 (d, | |
| | J = 4.8 Hz, 3H), 2.32 (s, 3H). | |
| 10 | 10.73 (s, 1H), 9.45 (d, J = 2.1 Hz, | 493.2 |
| | 1H), 9.42 (s, 1H), 9.25 (s, 1H), | |
| | 9.07 (q, J = 4.8 Hz, 1H), 8.80 (d, | |
| | J = 2.0 Hz, 1H), 8.66 (s, 1H), 8.62 | |
| | (m, 1H), 8.57 (m, 1H), 8.10 (d, J = | |
| | 8.2 Hz, 1H), 7.78 (m, 2H), 7.40 | |
| | (d, J = 8.1 Hz, 1H), 2.86 (d, J = | |
| | 4.8 Hz, 3H), 2.28 (s, 3H). | |
| 11 | 10.70 (s, 1H), 9.25 (s, 1H), 9.08 | 478.0 |
| | (d, J = 2.1 Hz, 1H), 8.74 (q, J = | |
| | 5.1 Hz, 1H), 8.72 (d, J = 2.0 Hz, | |
| | 1H), 8.57 (d, J = 2.2 Hz, 1H), | |
| | 8.40 (s, 1H), 8.27 (m, 1H), 8.20 | |
| | (s, 1H), 8.10 (d, J = 8.2 Hz, 1H), | |
| | 8.08 (dd, J = 1.8, 5.1 Hz, 1H), | |
| | 7.72-7.82 (m, 3H), 7.39 (d, J = | |
| | 8.3 Hz, 1H), 2.29 (s, 3H). | |
| 12 | 10.69 (s, 1H), 9.38 (d, J = 2.1 Hz, | 492.0 |
| | 1H), 9.18 (s, 1H), 8.81-8.88 (m, | |
| | 2H), 8.77 (s, 1H), 8.75 (q, J = 5.1 | |
| | Hz, 1H), 8.69 (s, 1H), 8.33 (dd, J = | |
| | 1.9, 5.2 Hz, 1H), 8.19 (s, 1H), | |
| | 7.81 (dd, J = 2.2, 8.3 Hz, 1H), | |
| | 7.75 (d, J = 2.3 Hz, 1H), 7.56 (dd, | |
| | J = 1.6, 5.0 Hz, 1H), 7.40 (d, J = | |
| | 8.3 Hz, 1H), 2.85 (d, J = 4.8 Hz, | |
| | 3H), 2.29 (s, 3H). | |
| 13 | 10.67 (s, 1H), 9.37 (d, J = 2.0 Hz, | 506.2 |
| | 1H), 9.18 (d, J = 1.0 Hz, 1H), | |
| | 8.84 (q, J = 4.8 Hz, 1H), 8.75 (m, | |
| | 1H), 8.73 (d, J = 0.8 Hz, 1H), | |
| | 8.69 (s, 1H), 8.30 (dd, J = 1.8, 5.1 | |
| | Hz, 1H), 7.98 (s, 1H), 7.80 (dd, | |
| | J = 2.3, 8.1 Hz, 1H), 7.73 (d, J = | |
| | 2.4 Hz, 1H), 7.41 (s, 1H), 7.39 (d, | |
| | J = 8.4 Hz, 1H), 2.85 (d, J = 4.9 | |
| | Hz, 3H), 2.66 (s, 3H), 2.29 (s, | |
| | 3H). | |
| 14 | 10.63 (s, 1H), 9.22 (s, 1H), 8.98 | 488.2 |
| | (s, 1H), 8.84 (q, J = 5.0 Hz, 2H), | |
| | 8.74 (m, 2H), 8.48 (s, 1H), 8.33 | |
| | (dd, J = 1.8, 5.3 Hz, 1H), 8.19 (s, | |
| | 1H), 7.81 (dd, J = 2.1, 8.0 Hz, | |
| | 1H), 7.75 (d, J = 2.3 Hz, 1H), | |
| | 7.55 (d, J = 5.0 Hz, 1H), 7.39 (d, | |
| | J = 8.3 Hz, 1H), 2.84 (d, J = 4.8 | |
| | Hz, 3H), 2.29 (s, 3H), 2.08 (t, J = | |
| | 19.1 Hz, 3H). | |
| 15 | 10.63 (s, 1H), 9.22 (d, J = 2.1 Hz, | 502.2 |
| | 1H), 8.98 (s, 1H), 8.85 (q, J = 5.1 | |
| | Hz, 1H), 8.69-8.77 (m, 2H), 8.49 | |
| | (s, 1H), 8.30 (dd, J = 1.8, 5.2 Hz, | |
| | 1H), 7.99 (s, 1H), 7.80 (dd, J = | |
| | 2.3, 8.3 Hz, 1H), 7.74 (d, J = 2.3 | |
| | Hz, 1H), 7.34-7.43 (m, 2H), 2.84 | |
| | (d, J = 4.8 Hz, 3H), 2.66 (s, 3H), | |
| | 2.28 (s, 3H), 2.08 (t, J = 19.2 Hz, | |
| | 3H). | |
| 16 | 11.30 (s, 1H), 9.94 (d, J = 2.2 Hz, | 493.2 |
| | 1H), 9.07 (d, J = 2.1 Hz, 1H), | |
| | 8.84 (q, J = 5.0 Hz, 1H), 8.74 (d, | |
| | J = 5.2 Hz, 2H), 8.56 (d, J = 2.2 | |
| | Hz, 1H), 8.38 (d, J = 1.9 Hz, 1H), | |
| | 8.28 (t, J = 2.0 Hz, 1H), 8.06 (dd, | |
| | J = 1.9, 5.2 Hz, 1H), 7.96 (m, | |
| | 2H), 7.39 (d, J = 8.9 Hz, 1H), | |
| | 2.84 (dd, J = 1.4, 4.9 Hz, 3H), | |
| | 2.30 (s, 3H). | |
| 17 | 11.31 (s, 1H), 9.94 (s, 1H), 9.39- | 494.2 |
| | 9.45 (m, 2H), 9.05 (m, 1H), 8.81 | |
| | (s, 1H), 8.65 (s, 1H), 8.62 (s, 1H), | |
| | 8.56 (s, 1H), 7.92-7.99 (m, 2H), | |
| | 7.39 (d, J = 8.2 Hz, 1H), 2.85 (d, | |
| | J = 4.8 Hz, 3H), 2.29 (s, 3H). | |
| 18 | 11.29 (s, 1H), 9.94 (d, J = 2.1 Hz, | 552.2 |
| | 1H), 8.79 (q, J = 5.0 Hz, 1H), | |
| | 8.67 (d, J = 5.1 Hz, 1H), 8.64 (s, | |
| | 1H), 8.56 (s, 1H), 8.20 (dd, J = | |
| | 1.7, 5.2 Hz, 1H), 7.87-7.94 (m, | |
| | 2H), 7.35 (d, J = 8.3 Hz, 1H), | |
| | 7.24 (s, 1H), 6.91 (t, J = 5.6 Hz, | |
| | 1H), 6.61 (s, 1H), 4.74 (m, 1H), | |
| | 3.63 (m, 2H), 3.49 (m, 2H), 2.83 | |
| | (d, J = 4.8 Hz, 3H), 2.28 (s, 3H). | |
| 20 | 9.87 (d, J = 2.0 Hz, 1H), 8.87 (q, | 192.1 |
| | J = 4.8 Hz, 1H), 8.83 (q, J = 5.0 | |
| | Hz, 1H), 8.76 (m, 2H), 8.71 (d, | |
| | J = 1.3 Hz, 1H), 8.33 (dd, J = 1.7, | |
| | 5.1 Hz, 1H), 8.24 (s, 1H), 7.60 | |
| | (dd, J = 1.2, 5.0 Hz, 1H), 7.37 (d, | |
| | J = 8.1 Hz, 1H), 7.07-7.35 (br m, | |
| | 2H), 7.01 (m, 2H), 2.85 (d, J = | |
| | 4.8 Hz, 3H), 2.30 (s, 3H). | |
| 21 | 9.16 (d, J = 2.2 Hz, 1H), 8.83 (q, | 464.2 |
| | J = 4.8 Hz, 1H), 8.82 (q, J = 5.2 | |
| | Hz, 1H), 8.76 (m, 2H), 8.32 (dd, | |
| | J = 2.0, 5.2 Hz, 1H), 8.21 (s, 1H), | |
| | 8.09 (d, J = 2.2 Hz, 1H), 7.58 (dd, | |
| | J = 1.4, 5.0 Hz, 1H), 7.35 (d, J = | |
| | 8.0 Hz, 1H), 6.93-7.00 (m, 2H), | |
| | 6.88 (br s, 1H), 2.86 (d, J = 4.8 | |
| | Hz, 3H), 2.29 (s, 3H), 2.13 (m, | |
| | 1H), 1.14-1.24 (m, 2H), 0.94-1.03 | |
| | (m, 2H), one NH is under | |
| | solvents. | |
| 26 | 11.06 (s, 1H), 9.25 (d, J = 2.1 Hz, | 465.2 |
| | 1H), 8.83 (q, J = 5.1 Hz, 2H), | |
| | 8.73 (m, 2H), 8.32 (dd, J = 1.7, | |
| | 5.2 Hz, 1H), 8.18 (s, 1H), 7.94 | |
| | (m, 3H), 7.55 (dd, J = 1.5, 5.0 Hz, | |
| | 1H), 7.36 (d, J = 8.1 Hz, 1H), | |
| | 2.84 (d, J = 4.8 Hz, 3H), 2.29 (s, | |
| | 3H), 2.12 (m, 1H), 1.21 (m, 2H), | |
| | 1.02 (m, 2H). | |
| 27 | 11.02 (s, 1H), 9.25 (d, J = 2.3 Hz, | 524.2 |
| | 1H), 8.75-8.83 (m, 1H), 8.67 (q, | |
| | J = 5.1 Hz, 1H), 8.64 (s, 1H), 8.17- | |
| | 8.23 (m, 1H), 7.84-7.95 (m, 3H), | |
| | 7.32 (d, J = 8.2 Hz, 1H), 7.23 (s, | |
| | 1H), 6.89 (t, J = 5.7 Hz, 1H), 6.60 | |
| | (s, 1H), 4.75 (t, J = 5.3 Hz, 1H), | |
| | 3.63 (m, 2H), 3.49 (m, 2H), 2.83 | |
| | (d, J = 4.8 Hz, 3H), 2.27 (s, 3H), | |
| | 2.08-2.19 (m, 1H), 1.12-1.22 (m, | |
| | 2H), 0.98-1.06 (m, 2H). | |
| 29 | 11.29 (s, 1H), 9.94 (d, J = 2.1 Hz, | 566.2 |
| | 1H), 8.79 (q, J = 5.2 Hz, 1H), | |
| | 8.67 (d, J = 5.1 Hz, 1H), 8.63 (s, | |
| | 1H), 8.55 (s, 1H), 8.20 (m, 1H), | |
| | 7.87-7.95 (m, 2H), 7.35 (d, J = | |
| | 8.1 Hz, 1H), 7.22 (s, 1H), 6.68 (s, | |
| | 1H), 6.60 (s, 1H), 4.14 (s, 1H), | |
| | 3.55 (m, 1H), 3.43 (m, 1H), 2.83 | |
| | (d, J = 4.8 Hz, 3H), 2.29 (s, 3H), | |
| | 1.21 (d, J = 6.7 Hz, 3H), 0.84 (t, | |
| | J = 6.6 Hz, 1H). | |
| 30 | 11.24 (s, 1H), 9.72 (d, J = 2.2 Hz, | 489.2 |
| | 1H), 9.06 (d, J = 2.1 Hz, 1H), | |
| | 8.84 (q, J = 5.1 Hz, 1H), 8.73 (m, | |
| | 2H), 8.38 (m, 2H), 8.28 (m, 1H), | |
| | 8.06 (m, 1H), 7.96 (m, 2H), 7.38 | |
| | (d, J = 8.9 Hz, 1H), 2.84 (d, J = | |
| | 4.8 Hz, 3H), 2.29 (s, 3H), 2.12 (t, | |
| | J = 19.2 Hz, 3H). | |
| 31 | 11.26 (s, 1H), 9.72 (s, 1H), 8.85 | 489.2 |
| | (m, 2H), 8.77 (s, 1H), 8.73 (d, J = | |
| | 4.4 Hz, 1H), 8.37 (s, 1H), 8.33 | |
| | (m, 1H), 8.19 (s, 1H), 7.97 (m, | |
| | 2H), 7.56 (m, 1H), 7.39 (d, J = | |
| | 8.5 Hz, 1H), 2.84 (d, J = 4.8 Hz, | |
| | 3H), 2.30 (s, 3H), 2.10 (t, J = 19.5 | |
| | Hz, 3H). | |
| 32 | 11.02 (s, 1H), 9.25 (d, J = 2.2 Hz, | 538.2 |
| | 1H), 8.79 (q, J = 4.8 Hz, 1H), | |
| | 8.67 (d, J = 5.2 Hz, 1H), 8.63 (s, | |
| | 1H), 8.19 (d, J = 5.2 Hz, 1H), | |
| | 7.81-7.93 (m, 3H), 7.87 (m, 1H), | |
| | 7.32 (d, J = 8.3 Hz, 1H), 7.21 (s, | |
| | 1H), 6.66 (d, J = 7.6 Hz, 1H), | |
| | 6.58 (s, 1H), 4.74 (br t, J = 5.6 | |
| | Hz, 1H), 4.15 (m, 1H), 3.54 (m, | |
| | 1H), 3.43 (m, 1H), 2.83 (d, J = | |
| | 4.8 Hz, 3H), 2.28 (s, 3H), 2.13 | |
| | (m, 1H), 1.21 (m, 6H), 0.97-1.07 | |
| | (m, 2H). | |
| 36 | 11.19 (s, 1H), 9.63 (d, J = 1.4 Hz, | 458.1 |
| | 1H), 8.80-8.87 (m, 2H), 8.77 (s, | |
| | 1H), 8.73(m, 1H), 8.42 (m, 1H), | |
| | 8.29-8.37 (m, 1H), 8.18 (s, 1H), | |
| | 7.91-7.98 (m, 2H), 7.55 (d, J = | |
| | 1.5 Hz, 1H), 7.38 (d, J = 8.2 Hz, | |
| | 1H), 2.84 (d, J = 4.8 Hz, 3H), | |
| | 2.29 (s, 3H). | |
| 37 | 11.33 (s, 1H), 9.95 (s, 1H), 8.81- | 507.2 |
| | 8.89 (m, 2H), 8.78 (s, 1H), 8.76 | |
| | (d, J = 2.3 Hz, 1H), 8.57 (s, 1H), | |
| | 8.33 (dd, J = 1.8, 5.1 Hz, 1H), | |
| | 8.18 (s, 1H), 8.01 (dd, J = 2.3, 8.4 | |
| | Hz, 1H), 7.93 (d, J = 2.3 Hz, 1H), | |
| | 7.54 (d, J = 5.0 Hz, 1H), 7.46 (d, | |
| | J = 8.5 Hz, 1H), 2.86 (d, J = 4.8 | |
| | Hz, 3H), 2.62 (q, J = 7.7 Hz, 2H), | |
| | 1.07 (t, J = 7.5 Hz, 3H). | |
| 38 | 11.27 (s, 1H), 9.75 (d, J = 2.0 Hz, | 562.2 |
| | 1H), 8.83 (q, J = 4.5 Hz, 1H), | |
| | 8.70 (d, J = 5.2 Hz, 1H), 8.66 (s, | |
| | 1H), 8.40 (d, J = 1.6 Hz, 1H), | |
| | 8.23 (dd, J = 1.6, 5.2 Hz, 1H), | |
| | 7.92-7.95 (m, 2H), 7.37 (d, J = | |
| | 8.0 Hz, 1H), 7.25 (s, 1H), 6.72 (d, | |
| | J = 7.6 Hz, 1H), 6.62 (s, 1H), 4.79 | |
| | (t, J = 5.6 Hz, 1H), 4.17 (br m, | |
| | 1H), 3.57 (m, 1H), 3.45 (m, 1H), | |
| | 2.85 (d, J = 4.8 Hz, 3H), 2.31 (s, | |
| | 3H), 2.13 (t, J = 19.6 Hz, 3H), | |
| | 1.23 (d, J = 6.8 Hz, 3H). | |
| 42 | 11.25 (s, 1H), 9.74 (d, J = 2.0 Hz, | 548.3 |
| | 1H), 8.82 (q, J = 4.8 Hz, 1H), | |
| | 8.70 (d, J = 5.2 Hz, 1H), 8.66 (s, | |
| | 1H), 8.39 (s, 1H), 8.23 (dd, J = | |
| | 1.6, 5.2 Hz, 1H), 7.90-7.96 (m, | |
| | 2H), 7.37 (d, J = 9.0 Hz, 1H), | |
| | 7.26 (s, 1H), 6.93 (t, J = 5.6 Hz, | |
| | 1H), 6.64 (s, 1H), 4.77 (t, J = 5.4 | |
| | Hz, 1H), 3.66 (q, J = 5.6 Hz, 2H), | |
| | 3.52 (q, J = 5.6 Hz, 2H), 2.86 (d, | |
| | J = 4.8 Hz, 3H), 2.31 (s, 3H), 2.12 | |
| | (t, J = 19.6 Hz, 3H). | |
| 43 | 11.27 (s, 1H), 9.75 (d, J = 2.0 Hz, | 549.0 |
| | 1H), 8.85 (d, J = 4.9 Hz, 1H), | |
| | 8.75 (d, J = 5.2 Hz, 1H), 8.72 (s, | |
| | 1H), 8.40 (s, 1H), 8.32 (dd, J = | |
| | 1.7, 5.2 Hz, 1H), 7.97-8.01 (m, | |
| | 2H), 7.82 (s, 1H), 7.40 (d, J = 9.0 | |
| | Hz, 1H), 6.96 (s, 1H), 4.93 (t, J = | |
| | 5.4 Hz, 1H), 4.52 (t, J = 5.1 Hz, | |
| | 2H), 3.84 (q, J = 5.2 Hz, 2H), | |
| | 2.87 (d, J = 4.8 Hz, 3H), 2.33 (s, | |
| | 3H), 2.13 (t, J = 19.4 Hz, 3H). | |
| 44 | 10.68 (s, 1H), 9.26 (d, J = 1.6 Hz, | 565.2 |
| | 1H), 8.82 (q, J = 4.6 Hz, 1H), | |
| | 8.69 (d, J = 5.0 Hz, 1H), 8.65 (d, | |
| | J = 1.0 Hz, 1H), 8.58 (dd, J = 1.8, | |
| | 8.2 Hz, 1H), 8.22 (dd, J = 1.8, 5.0 | |
| | Hz, 1H), 8.12 (d, J = 8.2 Hz, 1H), | |
| | 7.72-7.79 (m, 2H), 7.36 (d, J = | |
| | 8.0 Hz, 1H), 7.23 (s, 1H), 6.71 (d, | |
| | J = 7.6 Hz, 1H), 6.60 (s, 1H), | |
| | 4.72-4.84 (t, J = 5.4 Hz, 1H), 4.16 | |
| | (m, 1H), 3.57 (m, 1H), 3.47 (m, | |
| | 1H), 2.85 (d, J = 4.8 Hz, 3H), | |
| | 2.30 (s, 3H), 1.23 (d, J = 6.6 Hz, | |
| | 3H). | |
| 45 | 10.82 (s, 1H), 9.51 (s, 2H), 8.83 | 566.2 |
| | (q, J = 4.4 Hz, 1H), 8.70 (d, J = | |
| | 4.8 Hz, 1H), 8.64 (d, J = 0.8 Hz, | |
| | 1H), 8.23 (dd, J = 1.6, 5.2 Hz, | |
| | 1H), 7.72-7.76 (m, 2H), 7.39 (d, | |
| | J = 8.0 Hz, 1H), 7.23 (s, 1H), 6.73 | |
| | (d, J = 7.6 Hz, 1H), 6.60 (s, 1H), | |
| | 4.79 (t, J = 5.4 Hz, 1H), 4.17 (m, | |
| | 1H), 3.57 (m, 1H), 3.45 (m, 1H), | |
| | 2.85 (d, J = 5.2 Hz, 3H), 2.31 (s, | |
| | 3H), 1.23 (d, J = 6.8 Hz, 3H). | |

### Preparation of Example 3: 4-(2-methyl-5-(5-(trifluoromethyl)pyridazine-3-carboxamido)phenyl)-[2,4′-bipyridine]-2′-carboxamide

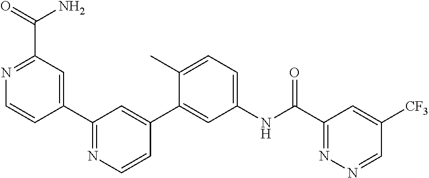 [SMILES: Cc1ccc(NC(=O)c2cc(C(F)(F)F)cnn2)cc1-c1ccnc(-c2ccnc(C(N)=O)c2)c1]

A mixture of N-(3-(2′-cyano-[2,4′-bipyridin]-4-yl)-4-methylphenyl)-5-(trifluoromethyl)pyridazine-3-carboxamide (1, 0.29 g, 0.64 mmol) and K₂CO₃ (0.13 g, 0.96 mmol) in DMSO (3 mL) was allowed to stir at rt. H₂O₂(1-2 drops, 30% in water) was added slowly and the reaction was stirred at rt for 2 h. The reaction was diluted with EtOAc (300 mL) and cooled to 0° C. under an ice bath. Sodium thiosulfate solution (1000 aq) was added slowly and the mixture was allowed to stir at 0° C. for 1 h. Resulting white solid was filtered, washed with water, and dried under high vacuum. The crude product was purified using silica gel column chromatography (0 to 100% EtOAc/hexanes). The product was contained impurities so the product was suspended in MeCN and sonicated. The solid was filtered, washed with MeCN, and dried. The product was indicated as 81% purity. The product was suspended in MeOH (5 mL) and sonicated. The solid was filtered, washed with MeOH, and dried to afford 4-(2-methyl-5-(5-(trifluoromethyl)pyridazine-3-carboxamido)phenyl)-[2,4′-bipyridine]-2′-carboxamide (3, 0.15 g, 49%) as white solid. ¹H NMR (400 MHz, DMSO-d₆): δ 11.35 (s, 1H), 9.95 (d, J=2.3 Hz, 1H), 8.84 (d, J=5.0 Hz, 1H), 8.78 (d, J=1.8 Hz, 1H), 8.75 (d, J=5.1 Hz, 1H), 8.57 (d, J=2.3 Hz, 1H), 8.33 (dd, J=1.9, 5.1 Hz, 1H), 8.20 (d, J=2.6 Hz, 2H), 7.99 (m, 2H), 7.74 (m, 1H), 7.57 (dd, J=1.6, 4.9 Hz, 1H), 7.40 (d, J=9.0 Hz, 1H), 2.31 (s, 3H); MS (ESI) m/z: 461.0 (M+H⁺).

The following compounds are prepared essentially by method of preparation Example 3.

| Ex | | | Yield | ¹H NMR (400 MHz, | MS (m/z: |
|---|---|---|---|---|---|
| No | SM | Product | (%) | DMSO-d₆):□ | M + H⁺) |
| 7 | 6 H₂O₂ | 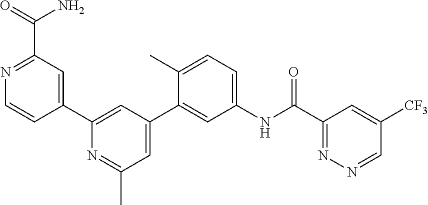 [SMILES: Cc1cc(-c2cc(NC(=O)c3cc(C(F)(F)F)cnn3)ccc2C)cc(-c2ccnc(C(N)=O)c2)n1] | 30 | 11.34 (s, 1H), 9.96 (d, J = 2.2 Hz, 1H), 8.78 (s, 1H), 8.73 (d, J = 5.1 Hz, 1H), 8.57 (s, 1H), 8.31 (m, 1H), 8.20 (s, 1H), 8.00 (s, 1H), 7.94-7.99 (m, 2H), 7.73 (s, 1H), 7.43 (s, 1H), 7.40 (d, J = 8.9 Hz, 1H), 2.66 (s, 3H), 2.31 (s, 3H). | 493.2 |

### Preparation of Example 52: N³-(3-(6-(2-hydroxyethoxy)-2′-(methylcarbamoyl)-[2,4′-bipyridin]-4-yl)-4-methylphenyl)pyridazine-3,5-dicarboxamide

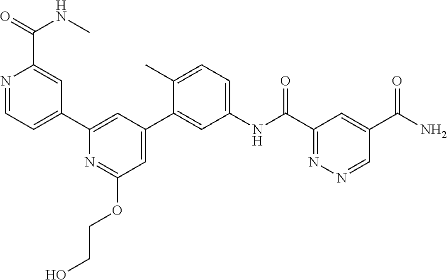 [SMILES: CNC(=O)c1cc(-c2cc(-c3cc(NC(=O)c4cc(C(N)=O)cnn4)ccc3C)cc(OCCO)n2)ccn1]

To a solution of 5-cyano-N-[3-[2-(2-hydroxyethoxy)-6-[2-(methylcarbamoyl)-4-pyridyl]-4-pyridyl]-4-methyl-phenyl]pyridazine-3-carboxamide (49, 180 mg, crude) in a mixture of THF (2 mL) and H₂O (0.4 mL) was treated with NaOH (22 mg, 3 eq). The mixture was stirred at rt ° C. for 4 h. The reaction mixture was diluted with water (3 mL) and extracted with EtOAc (3×). The combined organic layers were washed with brine, dried over anhydrous Na₂SO₄, filtered, and concentrated under reduced pressure. The crude was purified by prep-HPLC (20 to 50% H₂O (10 mM NH₄HCO₃)/CH₃CN) to obtain N³-[3-[2-(2-hydroxyethoxy)-6-[2-(methylcarbamoyl)-4-pyridyl]-4-pyridyl]-4-methyl-phenyl]pyridazine-3,5-dicarboxamide (16 mg, 16%) as a pale-yellow solid. ¹H NMR (400 MHz, DMSO-d₆): δ 11.23 (s, 1H), 9.75 (d, J=2.0 Hz, 1H), 8.84 (q, J=4.4 Hz, 1H), 8.74 (d, J=5.2 Hz, 1H), 8.71 (d, J=1.2 Hz, 1H), 8.67 (br s, 1H), 8.65 (d, J=2.4 Hz, 1H), 8.31 (dd, J=1.6, 5.2 Hz, 1H), 8.10 (s, 1H), 7.96-8.00 (m, 2H), 7.81 (d, J=0.8 Hz, 1H), 7.39 (m, 1H), 6.96 (s, 1H), 4.92 (t, J=5.6 Hz, 1H), 4.50 (t, J=4.8 Hz, 2H), 3.83 (q, J=5.4 Hz, 2H), 2.85 (d, J=4.8 Hz, 3H), 2.32 (s, 3H); MS m/z: 528.2 (M+H⁺).

### Biochemical Assay for B-Raf

Activity of B-Raf kinase (SEQ ID NO: 1) was determined spectroscopically using a coupled pyruvate kinase/lactate dehydrogenase assay that continuously monitors the ATP hydrolysis-dependent oxidation of NADH (e.g., Schindler et al., Science, 2000, 289, 1938-1942). Assays were conducted in 384-well plates (100 μL final volume) using 0.13 nM B-Raf (Sigma), 1.5 units pyruvate kinase, 2.1 units lactate dehydrogenase, 1 mM phosphoenol pyruvate, 0.28 mM NADH, 30.1 nM MEK (SignalChem), and 1 mM ATP in assay buffer (100 mM Tris, pH 7.5, 15 mM MgCl₂, 0.5 mM DTT, 0.1% octyl-glucoside, 0.002% (w/v) BSA, and 0.002% Triton X-100). Inhibition of B-Raf was measured by adding serial diluted test compound (final assay concentration of 1% DMSO). A decrease in absorption at 340 nm was monitored continuously for 6 h at 30° C. on a multi-mode microplate reader (BioTek). The reaction rate was calculated using the 4 to 5 h time frame. The reaction rate at each concentration of compound was converted to percent inhibition using controls (i.e., reaction with no test compound and reaction with a known inhibitor) and IC₅₀ values were calculated by fitting a four-parameter sigmoidal curve to the data using Prism (GraphPad software).

B-Raf protein sequence residues 416-766 with N-
terminal GST-tag
(SEQ ID NO: 1)
LQKSPGPQRERKSSSSSEDRNRMKTLGRRDSSDDWEIPDGQITVGQRIG
SGSFGTVYKGKWHGDVAVKMLNVTAPTPQQLQAFKNEVGVLRKTRHVNI
LLFMGYSTKPQLAIVTQWCEGSSLYHHLHIIETKFEMIKLIDIARQTAQ
GMDYLHAKSIIHRDLKSNNIFLHEDLTVKIGDFGLATVKSRWSGSHQFE
QLSGSILWMAPEVIRMQDKNPYSFQSDVYAFGIVLYELMTGQLPYSNIN
NRDQIIFMVGRGYLSPDLSKVRSNCPKAMKRLMAECLKKKRDERPLFPQ
ILASIELLARSLPKIHRSASEPSLNRAGFQTEDFSLYACASPKTPIQAG
GYGAFPVH

### Biochemical Assay for C-Raf

Activity of C-Raf kinase (SEQ ID NO: 2) was determined spectroscopically using a coupled pyruvate kinase/lactate dehydrogenase assay that continuously monitors the ATP hydrolysis-dependent oxidation of NADH (e.g., Schindler et al., Science, 2000, 289, 1938-1942). Assays were conducted in 384-well plates (100 μL final volume) using 0.43 nM C-Raf (Sigma), 1.5 units pyruvate kinase, 2.1 units lactate dehydrogenase, 1 mM phosphoenol pyruvate, 0.28 mM NADH, 30.1 nM MEK (SignalChem), and 1 mM ATP in assay buffer (100 mM Tris, pH 7.5, 15 mM MgCl₂, 0.5 mM DTT, 0.1% octyl-glucoside, 0.002% (w/v) BSA, and 0.002% Triton X-100). Inhibition of C-Raf was measured by adding serial diluted test compound (final assay concentration of 1% DMSO). A decrease in absorption at 340 nm was monitored continuously for 6 h at 30° C. on a multi-mode microplate reader (BioTek). The reaction rate was calculated using the 4 to 5 h time frame. The reaction rate at each concentration of compound was converted to percent inhibition using controls (i.e., reaction with no test compound and reaction with a known inhibitor) and IC₅₀ values were calculated by fitting a four-parameter sigmoidal curve to the data using Prism (GraphPad software).

C-Raf residues 306-end; Y340D, Y341D with N-
terminal GST-tag
(SEQ ID NO: 2)
QPKTPVPAQRERAPVSGTQEKNKIRPRGQRDSSDDWEIEASEVMLSTRI
GSGSFGTVYKGKWHGDVAVKILKVVDPTPEQFQAFRNEVAVLRKTRHVN
ILLFMGYMTKDNLAIVTQWCEGSSLYKHLHVQETKFQMFQLIDIARQTA
QGMDYLHAKNIIHRDMKSNNIFLHEGLTVKIGDFGLATVKSRWSGSQQV
EQPTGSVLWMAPEVIRMQDNNPFSFQSDVYSYGIVLYELMTGELPYSHI
NNRDQIIFMVGRGYASPDLSKLYKNCPKAMKRLVADCVKKVKEERPLFP
QILSSIELLQHSLPKINRSASEPSLHRAAHTEDINACTLTTSPRLPVF

TABLE 1
Inhibition of biochemical activity of BRAF and CRAF
kinases by exemplary compounds (“Ex No”).

| Ex No | BRAF | CRAF |
|---|---|---|
| 2 | +++ | ++++ |
| 3 | ++++ | ++++ |
| 4 | +++ | ++++ |
| 5 | + | +++ |
| 7 | +++ | ++++ |
| 8 | ++++ | ++++ |
| 9 | ++++ | ++++ |
| 10 | +++ | +++ |
| 11 | ++ | +++ |
| 12 | ++++ | ++++ |
| 13 | +++ | ++++ |
| 14 | ++++ | ++++ |
| 15 | +++ | ++++ |
| 16 | ++++ | ++++ |
| 17 | +++ | +++ |
| 18 | ++++ | ++++ |
| 19 | ++++ | ++++ |
| 20 | ++ | +++ |
| 21 | ++ | +++ |
| 22 | ++++ | ++++ |
| 23 | ++++ | ++++ |
| 24 | ++++ | ++++ |
| 25 | ++++ | ++++ |
| 26 | ++++ | ++++ |
| 27 | ++++ | ++++ |
| 28 | ++++ | ++++ |
| 29 | ++++ | ++++ |
| 30 | ++++ | ++++ |
| 31 | ++++ | ++++ |
| 32 | ++++ | ++++ |
| 33 | +++ | ++++ |
| 34 | ++++ | ++++ |
| 35 | ++++ | ++++ |
| 36 | +++ | ++++ |
| 37 | + | + |
| 38 | ++++ | ++++ |
| 39 | +++ | +++ |
| 41 | + | + |
| 42 | ++++ | ++++ |
| 43 | ++++ | ++++ |
| 44 | +++ | +++ |
| 45 | +++ | +++ |
| 46 | +++ | +++ |
| 47 | ++++ | ++++ |
| 48 | ++++ | ++++ |
| 49 | ++++ | ++++ |
| 50 | ++++ | ++++ |
| 51 | +++ | ++++ |
| 52 | +++ | ++++ |
| 53 | ++++ | ++++ |

For Table 1, “++++” refers to an IC₅₀ less than or equal to 100 nM; “+++” refers to an IC₅₀ greater than 100 nM and less than or equal to 500 nM; “++” refers to an IC₅₀ greater than 500 nM and less than or equal to 1000 nM; and “+” refers to an IC₅₀ greater than 1000 nM and less than or equal to 10000 nM.

### MiaPaca-2 Cell Proliferation Assay

MiaPaca-2 cells (catalog #CRL-1420) are obtained from the American Type Culture Collect (ATTC, Manassas, VA). Briefly, cells were grown in DMVEM supplemented with 1000 characterized fetal bovine serum (Invitrogen, Carlsbad, CA), 2.5% New Zealand sourced horse serum and 1% Penicillin/Streptomycin/L-Glutamine at 37° C., 500 CO₂, and 9500 humidity. Cells are expanded until reaching 7095 confluency at which point they are sub-cultured or harvested for assay use. For assays completed in 96-well plates, a serial dilution of test compound is dispensed into a 96-well black clear bottom plate in triplicate. Three thousand cells are added per well in 200 μL complete growth medium in the 96-well plate. Plates are incubated for 67 to 72 h at 37° C., 5% CO₂, and 95% humidity. At the end of the incubation, 40 μL of a 440 mM solution of resazurin (Sigma, St. Louis, MO) in PBS is added to each well of the plate and plates are incubated for an additional 5 to 6 h at 37° C., 5% CO₂, and 95% humidity. For 384-well assays, a threefold, 8-point serial dilution of compound was spotted column wise into a 384 well black, clear bottom, tissue culture treated plate using the Beckman Coulter Echo 650. Likewise, a threefold, 8-point serial dilution of cobimetinib was spotted row wise into the 384 well black, clear bottom plate containing compound. DMSO concentration was constant across all wells at 0.4%. Seven hundred fifty cells are added per well in 50 μL complete growth medium in each 384-well plate. Plates are incubated for 67 to 72 h at 37° C., 5% CO₂, and 95% humidity. At the end of the incubation, 10 μL of a 440 μM solution of resazurin (Sigma, St. Louis, MO) in PBS is added to each well of the plate and plates are incubated for an additional 4 to 6 h at 37° C., 5% CO₂, and 95% humidity. Plates are read on a Synergy2 or equivalent reader (Biotek, Winooski VT) using an excitation of 540 nm and an emission of 600 nm. Data is analyzed using Prism software (Graphpad, San Diego, CA) to calculate IC₅₀ values.

### HCT-116 Cell Proliferation Assay

HCT-116 cells (catalog #CCL-247) are obtained from the American Type Culture Collect (ATCC, Manassas, VA). Briefly, cells are grown in McCoy's 5A supplemented with 10% characterized fetal bovine serum (Invitrogen, Carlsbad, CA) and 1% Penicillin/Streptomycin/L-Glutamine at 37° C., 5% CO₂, and 95% humidity. Cells are expanded until reaching 70-95% confluency at which point they are sub-cultured or harvested for assay use. A serial dilution of test compound is dispensed into a 384-well black clear bottom plate in triplicate. 375 cells are added per well in 50 μL complete growth medium in the 384-well plate. Plates are incubated for 67 to 72 h at 37° C., 5% CO₂, and 95% humidity. At the end of the incubation, 40 μL of a 440 mM solution of resazurin (Sigma, St. Louis, MO) in PBS is added to each well of the plate and plates are incubated for an additional 4 to 5 h at 37° C., 5% CO₂, and 95% humidity. Plates are read on a Synergy2 or equivalent reader (Biotek, Winooski, VT) using an excitation of 540 nm and an emission of 600 nm. Data is analyzed using Prism software (Graphpad, San Diego, CA) to calculate IC₅₀ values.

### HPAF-II Cell Proliferation Assay

HPAF-II cells (catalog #CRL-1997) are obtained from the American Type Culture Collect (ATCC, Manassas, VA). Briefly, cells are grown in Minimum Essential Media supplemented with 10% characterized fetal bovine serum (Invitrogen, Carlsbad, CA) and 1% Penicillin/Streptomycin/L-Glutamine at 37° C., 5% CO₂, and 95% humidity. Cells are expanded until reaching 70-95% confluency at which point they are sub-cultured or harvested for assay use. A serial dilution of test compound is dispensed into a 384-well black clear bottom plate in triplicate. 750 cells are added per well in 50 μL complete growth medium in the 384-well plate. Plates are incubated for 115 to 120 h at 37° C., 5% CO₂, and 95% humidity. At the end of the incubation, 40 μL of a 440 mM solution of resazurin (Sigma, St. Louis, MO) in PBS is added to each well of the plate and plates are incubated for an additional 18 to 24 h at 37° C., 5% CO₂, and 95% humidity. Plates are read on a Synergy2 or equivalent reader (Biotek, Winooski, VT) using an excitation of 540 nm and an emission of 600 nm. Data is analyzed using Prism software (Graphpad, San Diego, CA) to calculate IC₅₀ values.

### Pa16c Cell Proliferation Assay

Pa16c cells are obtained from Dr. Channing Der, University of North Carolina at Chapel Hill. Briefly, cells are grown in Dulbecco's Modified Eagle Medium supplemented with 10% characterized fetal bovine serum (Invitrogen, Carlsbad, CA) and 1% Penicillin/Streptomycin/L-Glutamine at 37° C., 5% CO₂, and 95% humidity. Cells are expanded until reaching 70-95% confluency at which point they are sub-cultured or harvested for assay use. A serial dilution of test compound is dispensed into a 384-well black clear bottom plate in triplicate. 750 cells are added per well in 50 μL complete growth medium in the 384-well plate. Plates are incubated for 67 to 72 h at 37° C., 5% CO₂, and 95% humidity. At the end of the incubation, 40 μL of a 440 mM solution of resazurin (Sigma, St. Louis, MO) in PBS is added to each well of the plate and plates are incubated for an additional 18 to 24 h at 37° C., 5% CO₂, and 95% humidity. Plates are read on a Synergy2 or equivalent reader (Biotek, Winooski, VT) using an excitation of 540 nm and an emission of 600 nm. Data is analyzed using Prism software (Graphpad, San Diego, CA) to calculate IC₅₀ values.

TABLE 2
Inhibition of cell proliferation in MiaPaca-2,
HCT-116, HPAF-II and Pa16c by
exemplary compounds (“Ex No”).

| Ex No | MiaPaca-2 | HCT-116 | HPAF-II | Pa16c |
|---|---|---|---|---|
| 2 | +++ | ++ | ++ | ++ |
| 3 | ++++ | ++++ | +++ | ++++ |
| 4 | +++ | +++ | +++ | + |
| 5 | ++ | +++ | | |
| 7 | +++ | +++ | +++ | +++ |
| 8 | ++ | +++ | + | + |
| 9 | +++ | +++ | +++ | +++ |
| 10 | + | | | |
| 11 | + | | | |
| 12 | + | + | + | + |
| 13 | + | + | + | + |
| 14 | + | ++ | + | |
| 15 | + | | | |
| 16 | ++ | ++ | + | + |
| 17 | + | + | + | + |
| 18 | ++++ | ++++ | +++ | ++++ |
| 19 | +++ | +++ | +++ | ++ |
| 20 | + | + | + | + |
| 21 | + | + | + | + |
| 22 | + | + | + | + |
| 23 | + | ++ | + | + |
| 24 | + | + | + | + |
| 25 | +++ | +++ | +++ | + |
| 26 | +++ | +++ | +++ | +++ |
| 27 | +++ | ++++ | +++ | ++++ |
| 28 | + | + | + | + |
| 29 | +++ | ++++ | +++ | +++ |
| 30 | + | ++ | + | + |
| 31 | +++ | +++ | +++ | +++ |
| 32 | +++ | +++ | +++ | +++ |
| 33 | + | + | + | + |
| 34 | ++ | +++ | ++ | +++ |
| 35 | +++ | +++ | +++ | +++ |
| 36 | ++ | ++ | + | + |
| 37 | + | + | + | + |
| 38 | +++ | +++ | +++ | +++ |
| 39 | + | + | + | + |
| 41 | ++ | +++ | +++ | +++ |
| 42 | ++++ | ++++ | +++ | +++ |
| 43 | ++ | +++ | +++ | +++ |
| 44 | + | + | + | + |
| 45 | + | + | + | + |
| 46 | + | ++ | + | + |
| 47 | ++++ | ++++ | ++++ | ++++ |
| 48 | ++ | +++ | +++ | + |
| 49 | ++ | ++ | ++ | ++++ |
| 50 | +++ | +++ | ++ | |
| 51 | ++ | ++ | + | |
| 52 | + | + | + | |
| 53 | +++ | +++ | +++ | |

For Table 2, “++++” refers to an IC₅₀ less than or equal to 100 nM; “+++” refers to an IC₅₀ greater than 100 nM and less than or equal to 500 nM; “++” refers to an IC₅₀ greater than 500 nM and less than or equal to 1000 nM; and “+” refers to an IC₅₀ greater than 1000 nM and less than or equal to 10000 nM.

### Biochemical Tubulin Polymerization Assay

Porcine Brain Tubulin (T240) and Tubulin Polymerization Assay Kit (BK011P) are purchased from Cytoskeleton, Inc (Denver, CO). Briefly, a serial dilution of test compound is dispensed into a 384-well black plate in triplicate. 25 μL of an assay mixture containing Buffer, Glycerol, GTP, and Porcine Brain Tubulin from the assay kit are added to each well of the 384-well plate. Plates are briefly centrifuged, then read on a Synergy Neo2 or equivalent reader (BioteK, Winooski, VT) using an excitation of 335 nm and an emission of 450 nm every 2 min for 1 h at 37° C. to generate kinetic data and maximum velocity of polymerization from 0 to 1 h. Data is analyzed using Prism software (Graphpad, San Diego, CA) to calculate IC₅₀values.

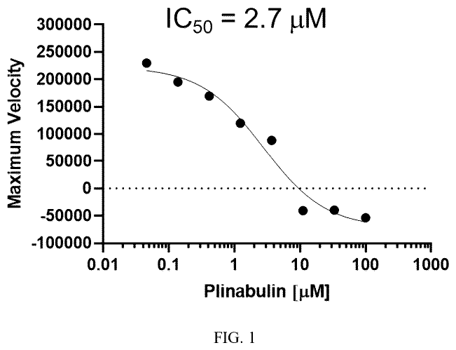

FIG. 1 represents the maximum velocity of tubulin polymerization in the presence of increasing concentrations of a tubulin depolymerizer plinabulin. In this recombinant biochemical tubulin assay, plinabulin inhibits tubulin polymerization with an IC₅₀ of 2.7 mM.

### Cellular Tubulin Polymerization Assay

HCT-116 cells (catalog #CCL-247) are obtained from the American Type Culture Collect (ATCC, Manassas, VA). Briefly, cells are grown in McCoy's 5A supplemented with 10% characterized fetal bovine serum (Invitrogen, Carlsbad, CA) and 1% Penicillin/Streptomycin/L-Glutamine at 37° C., 5% CO₂, and 95% humidity. Cells are expanded until reaching 70-95% confluency at which point they are sub-cultured or harvested for assay use. 450000 cells are added per well in 1500 μL complete growth medium in a tissue culture treated, 12-well plate. Plates are incubated for 18 to 24 h at 37° C., 5% CO₂, and 95% humidity. At the end of the incubation, 2000 μL of base culture media is added to each well, followed by a serial dilution of compound. Plates are incubated for an additional 1 h at 37° C., 5% CO₂, and 95% humidity. Lysates are generated by the addition of 100 μL Lysis and Microtubule Stabilization Buffer 1 (LMS01, Cytoskeleton Inc, Denver, CO) supplemented with GTP (BST06, Cytoskeleton Inc), ATP (BSA04, Cytoskeleton Inc), and Protease Inhibitor Cocktail Stock (PIC02, Cytoskeleton Inc). Cells are scraped off wells using a rubber cell scraper and collected in a clean 96-well plate. Lysates are centrifuged at 1000×g, 37° C. for 10 min and supernatant fraction is placed into an additional 96-well plate. The remaining pellet fraction is reconstituted in 100 μL LMS01 buffer and sonicated for 10 min. Pellet and Supernatant fractions are probed for a-tubulin via western blot analysis on the Jess System or equivalent (Bio-techne, Minneapolis, MN). Data is analyzed using Prism software (Graphpad, San Diego, CA) to calculate IC₅₀ values.

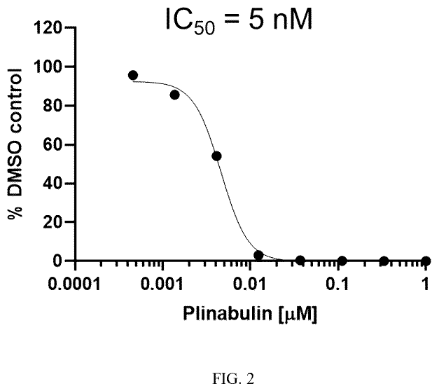

FIG. 2 represents the ratio of pellet (polymerized tubulin) to supernatant (tubulin dimers) compared to the DMSO control for increasing concentrations of a tubulin depolymerizer plinabulin. In this cellular tubulin assay, plinabulin inhibits tubulin polymerization with an IC₅₀ of 5 nM.

The compounds disclosed herein unexpectedly exhibit a dual mechanism of action by 1) inhibiting BRAF and CRAF MAP kinases, and 2) inhibiting tubulin polymerization. While inhibition of such dual mechanism may be achieved by combining several anti-cancer agents, the compounds disclosed herein provide such dual inhibition within the same pharmacophore. This unexpected dual mechanism of action enables single agent potent inhibition of mutant RAS cancer cell lines, not achievable with previously reported BRAF/CRAF inhibitors.

Representative examples are illustrated in Table 3. These examples inhibit BRAF and CRAF with biochemical IC₅₀ values as shown in Table 3. These examples also inhibit tubulin polymerization in the Tubulin Biochemical assay. In cellular assays, these examples exhibit potent single agent anti-proliferative activity in the MiaPaca-2 mutant KRAS pancreatic cancer cell line, potent single agent anti-proliferative activity in the HCT-116 mutant KRAS colorectal cancer cell line, and potent single agent anti-proliferative activity in the HPAF-II mutant KRAS pancreatic cancer cell line. In comparison, Table 4 contains representative compounds of the compounds disclosed in PCT/US2022/081242.

Other chemical classes of reported BRAF and/or CRAF inhibitors are shown in Table 5. While these compounds inhibit BRAF and/or CRAF, none of them potently inhibits tubulin polymerization. The compounds of Table 5 exhibit weaker anti-proliferative activity in the MiaPaca-2 mutant KRAS pancreatic cancer cell line, weaker anti-proliferative activity in the HCT-116 mutant KRAS colorectal cancer cell line, and weaker anti-proliferative activity in the HPAF-II mutant KRAS pancreatic cancer cell line.

TABLE 3
Representative Compounds

| Ex | | | | Tubulin | Tubulin | MiaPaca- | HCT- | HPAF- |
|---|---|---|---|---|---|---|---|---|
| No | Structure | BRAF | CRAF | Biochem | Cellular | 2 | 116 | II |
| 2 | 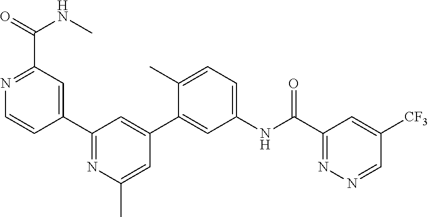 [SMILES: CNC(=O)c1cc(-c2cc(-c3cc(NC(=O)c4cc(C(F)(F)F)cnn4)ccc3C)cc(C)n2)ccn1] | +++ | ++++ | **** | +++ | +++ | ++ | ++ |
| 4 | 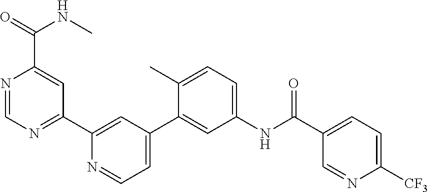 [SMILES: CNC(=O)c1cc(-c2cc(-c3cc(NC(=O)c4ccc(C(F)(F)F)nc4)ccc3C)ccn2)ncn1] | +++ | ++++ | *** | | +++ | +++ | +++ |
| 9 | 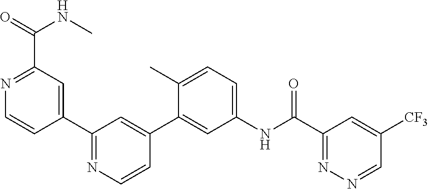 [SMILES: CNC(=O)c1cc(-c2cc(-c3cc(NC(=O)c4cc(C(F)(F)F)cnn4)ccc3C)ccn2)ccn1] | ++++ | ++++ | *** | +++ | +++ | +++ | +++ |
| 18 | 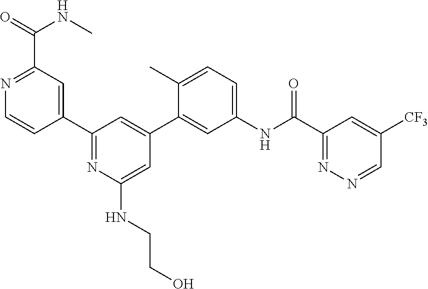 [SMILES: CNC(=O)c1cc(-c2cc(-c3cc(NC(=O)c4cc(C(F)(F)F)cnn4)ccc3C)cc(NCCO)n2)ccn1] | ++++ | ++++ | *** | ++++ | ++++ | ++++ | +++ |
| 26 | 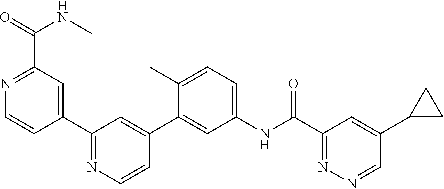 [SMILES: CNC(=O)c1cc(-c2cc(-c3cc(NC(=O)c4cc(C5CC5)cnn4)ccc3C)ccn2)ccn1] | ++++ | ++++ | **** | +++ | +++ | +++ | +++ |
| 27 | 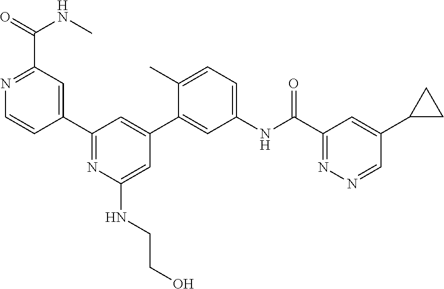 [SMILES: CNC(=O)c1cc(-c2cc(-c3cc(NC(=O)c4cc(C5CC5)cnn4)ccc3C)cc(NCCO)n2)ccn1] | ++++ | ++++ | *** | +++ | +++ | ++++ | +++ |

For Table 3, for BRAF, CRAF, MiaPaca-2. HCT-116, HPAF-JJ, and Tubulin cellular assay, “++++” refers to an IC₅₀ less than or equal to 100 nM; “+++” refers to an IC₅₀ greater than 100 nM and less than or equal to 500 nM; “C++” refers to an IC₅₀greater than 500 nM and less than or equal to 1000 nM; and “+” refers to an IC₅₀ greater than 1000 nM and less than or equal to 10000 nM; for Tubulin Biochemical assay, “****” refers to an IC₅₀ less than or equal to 3 mM; “***” refers to an IC₅₀ greater than 3 mM and less than or equal to 20 mM; “**” refers to an IC₅₀ greater than 20 mM and less than or equal to 100 mM; and “*” refers to an IC₅₀ greater than 100 mM.

TABLE 4
Representative Compounds from PCT/US2022/081242

| | | | Tubulin | Tubulin | | HCT- | HPAF- |
|---|---|---|---|---|---|---|---|
| | BRAF | CRAF | Biochem | Cellular | MiaPaca-2 | 116 | II |
| structure | IC₅₀ | IC₅₀ | IC₅₀ | IC₅₀ | IC₅₀ | IC₅₀ | IC₅₀ |
| 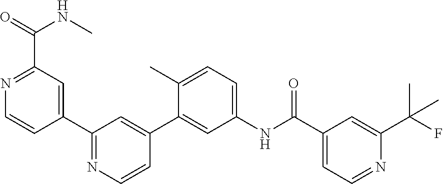 [SMILES: CNC(=O)c1cc(-c2cc(-c3cc(NC(=O)c4ccnc(C(C)(C)F)c4)ccc3C)ccn2)ccn1] | ++++ | ++++ | * | + | + | ++ | + |
| 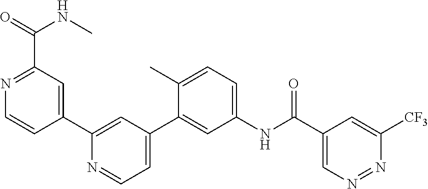 [SMILES: CNC(=O)c1cc(-c2cc(-c3cc(NC(=O)c4cnnc(C(F)(F)F)c4)ccc3C)ccn2)ccn1] | ++++ | ++++ | * | | + | + | + |
| 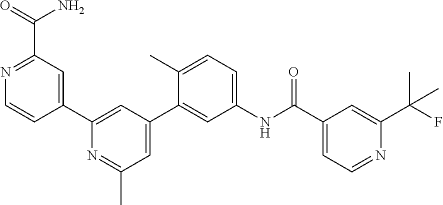 [SMILES: Cc1cc(-c2cc(NC(=O)c3ccnc(C(C)(C)F)c3)ccc2C)cc(-c2ccnc(C(N)=O)c2)n1] | ++++ | ++++ | * | | + | + | + |
| 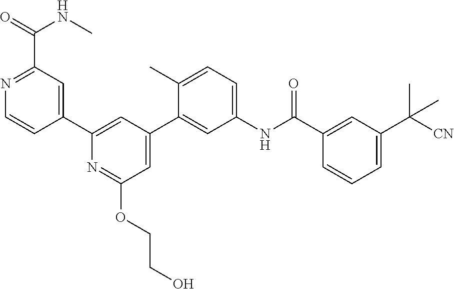 [SMILES: CNC(=O)c1cc(-c2cc(-c3cc(NC(=O)c4cccc(C(C)(C)C#N)c4)ccc3C)cc(OCCO)n2)ccn1] | ++++ | ++++ | | | + | +++ | ++ |
| 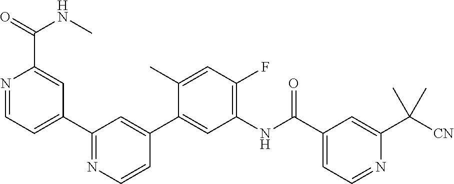 [SMILES: CNC(=O)c1cc(-c2cc(-c3cc(NC(=O)c4ccnc(C(C)(C)C#N)c4)c(F)cc3C)ccn2)ccn1] | +++ | ++++ | * | | + | ++ | + |

For Table 4, for BRAF, CRAF, MiaPaca-2. HCT-116, HPAF-JJ, and Tubulin cellular assay, “++++” refers to an IC₅₀ less than or equal to 100 nM; “+++” refers to an IC₅₀ greater than 100 nM and less than or equal to 500 nM; “C++” refers to an IC₅₀ greater than 500 nM and less than or equal to 1000 nM; and “+” refers to an IC₅₀ greater than 1000 nM and less than or equal to 10000 nM; for Tubulin Biochemical assay, “****” refers to an IC₅₀ less than or equal to 3 mM; “***” refers to an IC₅₀ greater than 3 mM and less than or equal to 20 mM; “**” refers to an IC₅₀ greater than 20 mM and less than or equal to 100 mM; and “*” refers to an IC₅₀ greater than 100 mM.

TABLE 5
Previously disclosed BRAF/CRAF inhibitors

| | | | Tubulin | Tubulin | MiaPaca- | HCT- | HPAF- |
|---|---|---|---|---|---|---|---|
| compound | BRAF | CRAF | Biochem | Cellular | 2 | 116 | II |
| 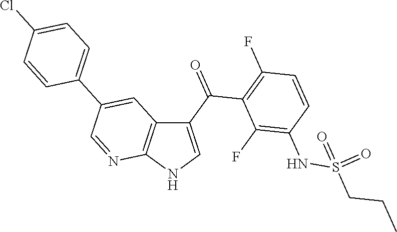 [SMILES: CCCS(=O)(=O)Nc1ccc(F)c(C(=O)c2c[nH]c3ncc(-c4ccc(Cl)cc4)cc23)c1F] | ++ | ++ | * | + | + | + | + |
| Vemurafenib | | | | | | | |
| 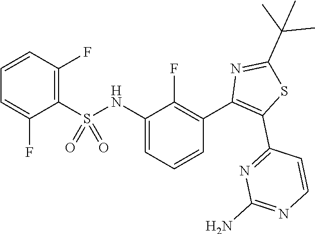 [SMILES: CC(C)(C)c1nc(-c2cccc(NS(=O)(=O)c3c(F)cccc3F)c2F)c(-c2ccnc(N)n2)s1] | ++++ | +++ | * | + | + | + | + |
| Dabrafenib | | | | | | | |
| 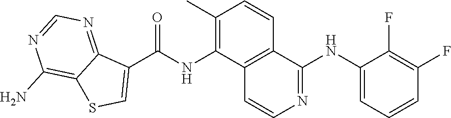 [SMILES: Cc1ccc2c(Nc3cccc(F)c3F)nccc2c1NC(=O)c1csc2c(N)ncnc12] | ++++ | ++++ | * | + | + | ++ | + |
| Belvarafenib | | | | | | | |
| 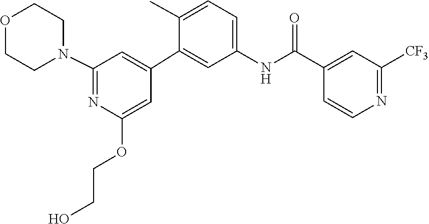 [SMILES: Cc1ccc(NC(=O)c2ccnc(C(F)(F)F)c2)cc1-c1cc(OCCO)nc(N2CCOCC2)c1] | ++++ | ++++ | * | + | + | + | + |
| Naporafenib | | | | | | | |
| 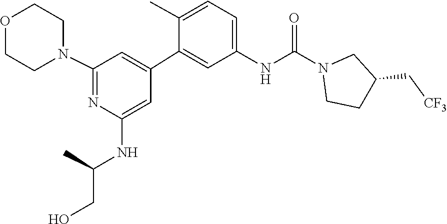 [SMILES: Cc1ccc(NC(=O)N2CC[C@@H](CC(F)(F)F)C2)cc1-c1cc(N[C@H](C)CO)nc(N2CCOCC2)c1] | +++ | ++++ | ** | + | + | ++ | + |
| Exarafenib | | | | | | | |
| 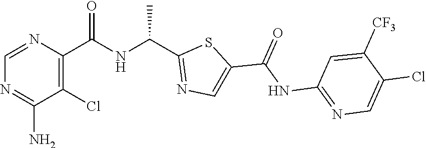 [SMILES: C[C@@H](NC(=O)c1ncnc(N)c1Cl)c1ncc(C(=O)Nc2cc(C(F)(F)F)c(Cl)cn2)s1] | ++ | +++ | * | + | + | + | + |
| Tovorafenib | | | | | | | |

For Table 5, for BRAF, CRAF, MiaPaca-2. HCT-116, HPAF-II, and Tubulin cellular assay, “++++” refers to an IC₅₀ less than or equal to 100 nM; “+++” refers to an IC₅₀ greater than 100 nM and less than or equal to 500 nM; “++” refers to an IC₅₀ greater than 500 nM and less than or equal to 1000 nM; and “+” refers to an IC₅₀ greater than 1000 nM and less than or equal to 10000 nM; for Tubulin Biochemical assay, “****” refers to an IC₅₀ less than or equal to 3 mM; “***” refers to an IC₅₀ greater than 3 mM and less than or equal to 20 mM; “**” refers to an IC₅₀ greater than 20 mM and less than or equal to 100 mM; and “*” refers to an IC₅₀ greater than 100 mM.

### EQUIVALENTS

While specific embodiments have been discussed, the above specification is illustrative and not restrictive. Many variations of the embodiments will become apparent to those skilled in the art upon review of this specification. The full scope of what is disclosed should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained.

What is claimed is:

1. A compound represented by Formula I-A: 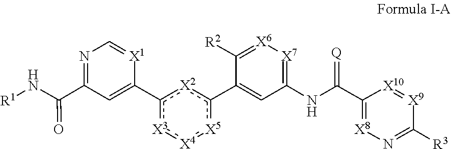
or a pharmaceutically acceptable salt, enantiomer, stereoisomer, or tautomer thereof, wherein:
Q is selected from the group consisting of O and NH;
X¹, X², and X⁵ are each independently selected from the group consisting of CH and N;
X³ and X⁴ are each independently selected from the group consisting of N, CH, C═O, C—O-L-E, C-L-E, C—N(R⁴)-L-E, and N-L-E;
X⁶ is selected from the group consisting of CH and N;
X⁷ is selected from the group consisting of CH, CF, and N;
X⁸ and X¹⁰ are each independently selected from the group consisting of CH, CF, and N;
X⁹ is selected from the group consisting of CR⁵ and N;
provided that not more than two of X², X³, X⁴, and X⁵ is N;
provided that not more than one of X⁶ and X⁷ is N;
provided that not more than one of X⁸, X⁹, and X¹⁰ is N;
provided that when X³ is N, X⁴ is C—O-L-E, C-L-E, C—N(R⁴)-L-E, N, or CH;
provided that when X⁴ is N, X³ is N, CH, C—O-L-E, C-L-E, or C—N(R⁴)-L-E;
provided that when X³ is C═O, X⁴ is N-L-E;
provided that when X⁴ is C═O, X³ is N-L-E;
R¹ is selected from the group consisting of H, alkyl, cycloalkyl, cycloalkylalkyl, alkoxyalkyl, hydroxyalkyl, heterocyclyl, heteroaryl, and haloalkyl,
wherein the alkyl substituent is independently optionally substituted, at each occurrence, with a substituent selected from the group consisting of amine, halogen, cyano, cycloalkyl, and heterocyclyl;
wherein the cycloalkyl of cycloalkyl or cycloalkylalkyl substituent is independently optionally substituted, at each occurrence, with a substituent selected from the group consisting of halogen and alkyl;
wherein the heterocyclyl substituent is independently optionally substituted, at each occurrence, with a substituent selected from the group consisting of halogen, and alkyl;
wherein the heteroaryl substituent is independently optionally substituted, at each occurrence, with a substituent selected from the group consisting of halogen, and alkyl;
R² is selected from the group consisting of alkyl, H, halogen, and alkoxy;
R³ is selected from the group consisting of H, haloalkyl, alkyl, cycloalkyl, and amine;
R⁴ is selected from the group consisting of H and alkyl;
R⁵ is selected from the group consisting of haloalkyl, cycloalkyl, cyano, H, alkyl, alkoxy, amine, amide, halogen, phosphine oxide, haloalkoxy, and cyanoalkyl, or wherein R⁵ taken together with R³ and the carbon atoms to which they are each attached form a cycloalkyl or a heterocyclyl ring having from 4 to 6 atoms in the ring structure;
L is selected from the group consisting of a direct bond and optionally substituted C₁-C₆alkyl; and
E is selected from the group consisting of H, alkyl, hydroxy, cycloalkyl, haloalkyl, alkoxy, haloalkoxy, alkoxylalkyl, amine, and optionally substituted heterocyclyl wherein the optionally substituted substituent, at each occurrence, is independently selected from the group consisting of alkyl, halogen, amine, hydroxy, oxo, and cyano.

2. A compound represented by Formula I-B: 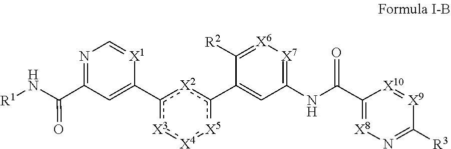
or a pharmaceutically acceptable salt, enantiomer, stereoisomer, or tautomer thereof, wherein:
X¹, X², and X⁵ are each independently selected from the group consisting of CH and N;
X³ and X⁴ are each independently selected from the group consisting of N, CH, C═O, C—O-L-E, C-L-E, C—N(R⁴)-L-E, and N-L-E;
X⁶ is selected from the group consisting of CH and N;
X⁷ is selected from the group consisting of CH, CF and N;
X⁸ and X¹⁰ are each independently selected from the group consisting of CH, CF, and N;
X⁹ is selected from the group consisting of CR⁵ and N;
provided that not more than two of X², X³, X⁴, and X⁵ is N;
provided that not more than one of X⁶ and X⁷ is N;
provided that not more than one of X⁸, X⁹, and X¹⁰ is N;
provided that when X³ is N, X⁴ is C—O-L-E, C-L-E, C—N(R⁴)-L-E, N, or CH;
provided that when X⁴ is N, X³ is N, CH, C—O-L-E, C-L-E, or C—N(R⁴)-L-E;
provided that when X³ is C═O, X⁴ is N-L-E;
provided that when X⁴ is C═O, X³ is N-L-E;
R¹ is selected from the group consisting of H, alkyl, cycloalkyl, alkoxyalkyl, hydroxyalkyl, heterocyclyl, heteroaryl, and haloalkyl,
wherein the alkyl substituent is independently optionally substituted, at each occurrence, with a substituent selected from the group consisting of amine, halogen, cyano, cycloalkyl, and heterocyclyl;
wherein the heterocyclyl substituent is independently optionally substituted, at each occurrence, with a substituent selected from the group consisting of halogen, and alkyl;
wherein the heteroaryl substituent is independently optionally substituted, at each occurrence, with a substituent selected from the group consisting of halogen, and alkyl;
R² is selected from the group consisting of alkyl, H, halogen, and alkoxy;
R³ is selected from the group consisting of H, haloalkyl, alkyl, cycloalkyl, amine;
R⁴ is selected from the group consisting of H and alkyl;
R⁵ is selected from the group consisting of haloalkyl, cycloalkyl, cyano, H, alkyl, alkoxy, amine, amide, halogen, phosphine oxide, haloalkoxy, and cyanoalkyl, or wherein R⁵ taken together with R³ and the carbon atoms to which they are each attached form a cycloalkyl or a heterocyclyl ring having from 4 to 6 atoms in the ring structure;
L is selected from the group consisting of a direct bond and optionally substituted C₁-C₆alkyl; and
E is selected from the group consisting of H, alkyl, hydroxy, cycloalkyl, haloalkyl, alkoxy, haloalkoxy, alkoxylalkyl, amine, and optionally substituted heterocyclyl wherein the optionally substituted substituent, at each occurrence, is independently selected from the group consisting of alkyl, halogen, amine, hydroxy, oxo, and cyano.

3. A compound represented by Formula I-C: 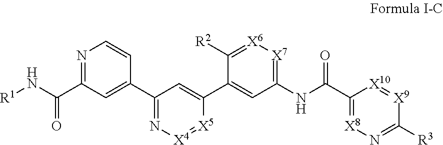
or a pharmaceutically acceptable salt, enantiomer, stereoisomer, or tautomer thereof, wherein:
X⁴ is selected from the group consisting of N, CH, C—O-L-E, C-L-E, and C—N(R⁴)-L-E;
X⁵ is selected from the group consisting of CH and N;
X⁶ is selected from the group consisting of CH and N;
X⁷ is selected from the group consisting of CH, CF and N;
X⁸ and X¹⁰ are each independently selected from the group consisting of CH, CF, and N;
X⁹ is selected from the group consisting of CR⁵ and N;
provided that not more than one of X⁴ and X⁵ is N;
provided that not more than one of X⁶ and X⁷ is N;
provided that not more than one of X⁸, X⁹, and X¹⁰ is N;
R¹ is selected from the group consisting of H, alkyl, cycloalkyl, alkoxyalkyl, hydroxyalkyl, heterocyclyl, heteroaryl, and haloalkyl,
wherein the alkyl substituent is independently optionally substituted, at each occurrence, with a substituent selected from the group consisting of amine, halogen, cyano, cycloalkyl, and heterocyclyl;
wherein the heterocyclyl substituent is independently optionally substituted, at each occurrence, with a substituent selected from the group consisting of halogen, and alkyl;
wherein the heteroaryl substituent is independently optionally substituted, at each occurrence, with a substituent selected from the group consisting of halogen, and alkyl;
R² is selected from the group consisting of alkyl, H, halogen, and alkoxy;
R³ is selected from the group consisting of H, haloalkyl, alkyl, cycloalkyl, amine;
R⁴ is selected from the group consisting of H and alkyl;
R⁵ is selected from the group consisting of haloalkyl, cycloalkyl, cyano, H, alkyl, alkoxy, amine, amide, halogen, phosphine oxide, haloalkoxy, and cyanoalkyl, or wherein R⁵ taken together with R³ and the carbon atoms to which they are each attached form a cycloalkyl or a heterocyclyl ring having from 4 to 6 atoms in the ring structure;
L is selected from the group consisting of a direct bond and optionally substituted C₁-C₆alkyl; and
E is selected from the group consisting of H, alkyl, hydroxy, cycloalkyl, haloalkyl, alkoxy, haloalkoxy, alkoxylalkyl, amine, and optionally substituted heterocyclyl wherein the optionally substituted substituent, at each occurrence, is independently selected from the group consisting of alkyl, halogen, amine, hydroxy, oxo, and cyano.

4. A compound represented by Formula I-D: 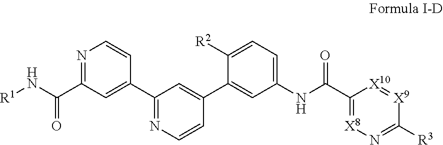
or a pharmaceutically acceptable salt, enantiomer, stereoisomer, or tautomer thereof, wherein:
X⁸ and X¹⁰ are each independently selected from the group consisting of CH, CF, and N;
X⁹ is selected from the group consisting of CR⁵ and N;
provided that not more than one of X⁸, X⁹, and X¹⁰ is N;
R¹ is selected from the group consisting of H, alkyl, cycloalkyl, alkoxyalkyl, hydroxyalkyl, heterocyclyl, heteroaryl, and haloalkyl,
wherein the alkyl substituent is independently optionally substituted, at each occurrence, with a substituent selected from the group consisting of amine, halogen, cyano, cycloalkyl, and heterocyclyl;
wherein the heterocyclyl substituent is independently optionally substituted, at each occurrence, with a substituent selected from the group consisting of halogen, and alkyl;
wherein the heteroaryl substituent is independently optionally substituted, at each occurrence, with a substituent selected from the group consisting of halogen, and alkyl;
R² is selected from the group consisting of alkyl, H, halogen, and alkoxy;
R³ is selected from the group consisting of H, haloalkyl, alkyl, cycloalkyl, amine;
R⁵ is selected from the group consisting of haloalkyl, cycloalkyl, cyano, H, alkyl, alkoxy, amine, amide, halogen, phosphine oxide, haloalkoxy, and cyanoalkyl, or wherein R⁵ taken together with R³ and the carbon atoms to which they are each attached form a cycloalkyl or a heterocyclyl ring having from 4 to 6 atoms in the ring structure.

5. The compound of claim 4, wherein R³ is H.

6. The compound of claim 4, wherein R⁵ is haloalkyl, cycloalkyl, or cyano.

7. A compound represented by Formula I-E: 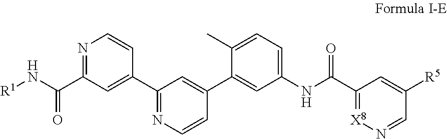
or a pharmaceutically acceptable salt, enantiomer, stereoisomer, or tautomer thereof, wherein:
X⁸ is selected from the group consisting of CH, CF, and N;
R¹ is selected from the group consisting of H, alkyl, cycloalkyl, alkoxyalkyl, hydroxyalkyl, heterocyclyl, heteroaryl, and haloalkyl,
wherein the alkyl substituent is independently optionally substituted, at each occurrence, with a substituent selected from the group consisting of amine, halogen, cyano, cycloalkyl, and heterocyclyl;
wherein the heterocyclyl substituent is independently optionally substituted, at each occurrence, with a substituent selected from the group consisting of halogen, and alkyl;
wherein the heteroaryl substituent is independently optionally substituted, at each occurrence, with a substituent selected from the group consisting of halogen, and alkyl;
and
R⁵ is selected from the group consisting of haloalkyl, cycloalkyl, cyano, H, alkyl, alkoxy, amine, amide, halogen, phosphine oxide, haloalkoxy, and cyanoalkyl.

8. A compound represented by Formula I-F: 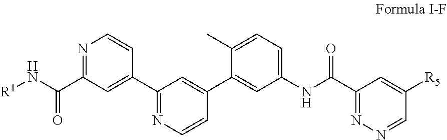
or a pharmaceutically acceptable salt, enantiomer, stereoisomer, or tautomer thereof, wherein:
R¹ is selected from the group consisting of H, alkyl, cycloalkyl, alkoxyalkyl, hydroxyalkyl, heterocyclyl, heteroaryl, and haloalkyl,
wherein the alkyl substituent is independently optionally substituted, at each occurrence, with a substituent selected from the group consisting of amine, halogen, cyano, cycloalkyl, and heterocyclyl;
wherein the heterocyclyl substituent is independently optionally substituted, at each occurrence, with a substituent selected from the group consisting of halogen, and alkyl;
wherein the heteroaryl substituent is independently optionally substituted, at each occurrence, with a substituent selected from the group consisting of halogen, and alkyl;
and
R⁵ is selected from the group consisting of haloalkyl, cycloalkyl, cyano, H, alkyl, alkoxy, amine, amide, halogen, phosphine oxide, haloalkoxy, and cyanoalkyl.

9. A compound of claim 8, wherein R¹ is alkyl, cycloalkyl, or H.

10. A compound of claim 8, wherein R⁵ is haloalkyl, cycloalkyl, or cyano.

11. A compound represented by Formula I-G: 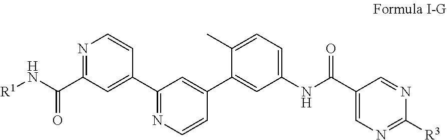
or a pharmaceutically acceptable salt, enantiomer, stereoisomer, or tautomer thereof, wherein:
R¹ is selected from the group consisting of H, alkyl, cycloalkyl, alkoxyalkyl, hydroxyalkyl, heterocyclyl, heteroaryl, and haloalkyl,
wherein the alkyl substituent is independently optionally substituted, at each occurrence, with a substituent selected from the group consisting of amine, halogen, cyano, cycloalkyl, and heterocyclyl;
wherein the heterocyclyl substituent is independently optionally substituted, at each occurrence, with a substituent selected from the group consisting of halogen, and alkyl;
wherein the heteroaryl substituent is independently optionally substituted, at each occurrence, with a substituent selected from the group consisting of halogen, and alkyl;
and
R³ is selected from the group consisting of H, haloalkyl, alkyl, cycloalkyl, and amine.

12. A compound of claim 11, wherein R³ is haloalkyl or cycloalkyl.

13. A compound represented by Formula I-H: 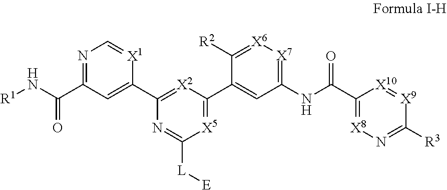
or a pharmaceutically acceptable salt, enantiomer, stereoisomer, or tautomer thereof, wherein:
X¹, X², and X⁵ are each independently selected from the group consisting of CH and N;
X⁶ is selected from the group consisting of CH and N;
X⁷ is selected from the group consisting of CH, CF and N;
X⁸ and X¹⁰ are each independently selected from the group consisting of CH, CF, and N;
X⁹ is selected from the group consisting of CR⁵ and N;
provided that not more than one of X² and X⁵ is N;
provided that not more than one of X⁶ and X⁷ is N;
provided that not more than one of X⁸, X⁹, and X¹⁰ is N;
R¹ is selected from the group consisting of H, alkyl, cycloalkyl, alkoxyalkyl, hydroxyalkyl, heterocyclyl, heteroaryl, and haloalkyl,
wherein the alkyl substituent is independently optionally substituted, at each occurrence, with a substituent selected from the group consisting of amine, halogen, cyano, cycloalkyl, and heterocyclyl;
wherein the heterocyclyl substituent is independently optionally substituted, at each occurrence, with a substituent selected from the group consisting of halogen, and alkyl;
wherein the heteroaryl substituent is independently optionally substituted, at each occurrence, with a substituent selected from the group consisting of halogen, and alkyl;
R² is selected from the group consisting of alkyl, H, halogen, and alkoxy;
R³ is selected from the group consisting of H, haloalkyl, alkyl, cycloalkyl, and amine;
R⁴ is selected from the group consisting of H and alkyl;
R⁵ is selected from the group consisting of haloalkyl, cycloalkyl, cyano, H, alkyl, alkoxy, amine, amide, halogen, phosphine oxide, haloalkoxy, and cyanoalkyl, or wherein R⁵ taken together with R³ and the carbon atoms to which they are each attached form a cycloalkyl or a heterocyclyl ring having from 4 to 6 atoms in the ring structure;
L is selected from the group consisting of a direct bond and optionally substituted C₁-C₆alkyl; and
E is selected from the group consisting of H, alkyl, hydroxy, cycloalkyl, haloalkyl, alkoxy, haloalkoxy, alkoxylalkyl, amine, and optionally substituted heterocyclyl wherein the optionally substituted substituent, at each occurrence, is independently selected from the group consisting of alkyl, halogen, amine, hydroxy, oxo, and cyano.

14. A compound represented by Formula I-J: 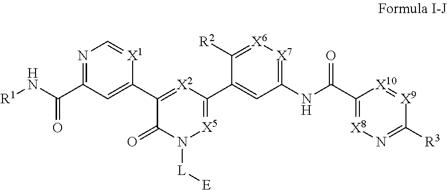
or a pharmaceutically acceptable salt, enantiomer, stereoisomer, or tautomer thereof, wherein:
X¹, X², and X⁵ are each independently selected from the group consisting of CH and N;
X⁶ is selected from the group consisting of CH and N;
X⁷ is selected from the group consisting of CH, CF and N;
X⁸ and X¹⁰ are each independently selected from the group consisting of CH, CF, and N;
X⁹ is selected from the group consisting of CR⁵ and N;
provided that not more than one of X² and X⁵ is N;
provided that not more than one of X⁶ and X⁷ is N;
provided that not more than one of X⁸, X⁹, and X¹⁰ is N;
R¹ is selected from the group consisting of H, alkyl, cycloalkyl, alkoxyalkyl, hydroxyalkyl, heterocyclyl, heteroaryl, and haloalkyl,
wherein the alkyl substituent is independently optionally substituted, at each occurrence, with a substituent selected from the group consisting of amine, halogen, cyano, cycloalkyl, and heterocyclyl;
wherein the heterocyclyl substituent is independently optionally substituted, at each occurrence, with a substituent selected from the group consisting of halogen, and alkyl;
wherein the heteroaryl substituent is independently optionally substituted, at each occurrence, with a substituent selected from the group consisting of halogen, and alkyl;
R² is selected from the group consisting of alkyl, H, halogen, and alkoxy;
R³ is selected from the group consisting of H, haloalkyl, alkyl, cycloalkyl, and amine;
R⁵ is selected from the group consisting of haloalkyl, cycloalkyl, cyano, H, alkyl, alkoxy, amine, amide, halogen, phosphine oxide, haloalkoxy, and cyanoalkyl, or wherein R⁵ taken together with R³ and the carbon atoms to which they are each attached form a cycloalkyl or a heterocyclyl ring having from 4 to 6 atoms in the ring structure;
L is selected from the group consisting of a direct bond and optionally substituted C₁-C₆alkyl; and
E is selected from the group consisting of H, alkyl, hydroxy, cycloalkyl, haloalkyl, alkoxy, haloalkoxy, alkoxylalkyl, amine, and optionally substituted heterocyclyl wherein the optionally substituted substituent, at each occurrence, is independently selected from the group consisting of alkyl, halogen, amine, hydroxy, oxo, and cyano.

15. The compound of any one of claims 1-2 and 13-14, wherein X¹ is CH.

16. The compound of any one of claims 1-2 and 13-15, wherein X² is CH.

17. The compound of any one of claims 1-2 and 15-16, wherein X³ is N.

18. The compound of any one of claims 1-3 and 15-17, wherein X⁴ is selected from the group consisting of N, CH, C—O-L-E, and C—N(R⁴)-L-E.

19. The compound of any one of claims 1-3 and 15-18, wherein X⁴ is selected from the group consisting of N and CH.

20. The compound of any one of claims 1-3 and 15-18, wherein X⁴ is selected from the group consisting of CO—CH(R⁴)—CH₂—OH and C—NH—CH(R⁴)—CH₂—OH.

21. The compound of any one of claims 1-3 and 13-20, wherein X⁵ is CH.

22. The compound of claim 1 or 2, wherein the ring containing X², X³, X⁴, and X⁵ is selected from the group consisting of: 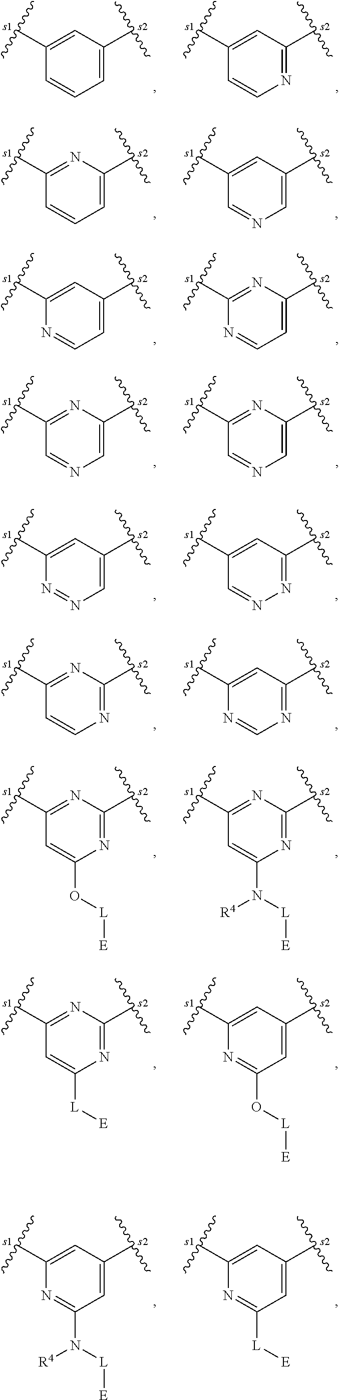 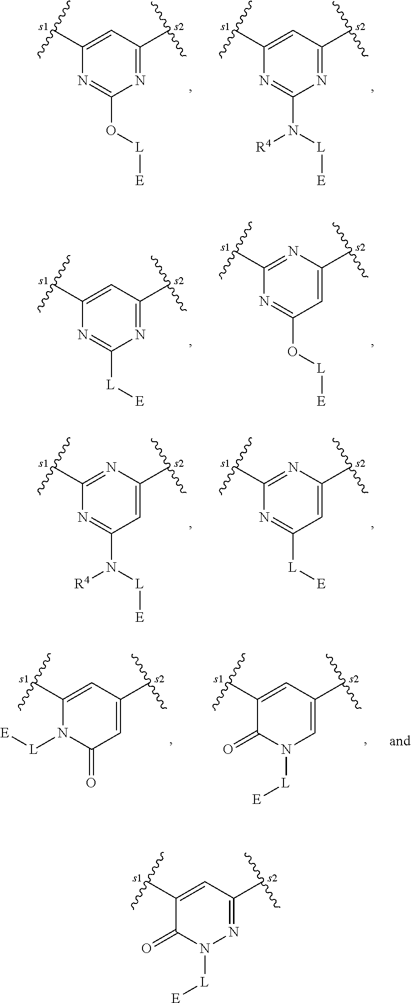
wherein s1 indicates attachment to ring comprising N and substituted with —C(O)NHR¹ and s2 indicates attachment to ring comprising X⁶ and X⁷.

23. The compound of any one of claims 1-3 and 13-22, wherein X⁶ is CH.

24. The compound of any one of claims 1-3 and 13-23, wherein X⁷ is CH.

25. The compound of any one of claims 1-7 and 13-24, wherein X⁸ is N.

26. The compound of any one of claims 1-6 and 13-25, wherein X⁹ is CRS.

27. The compound of any one of claims 1-10 and 13-26, wherein R⁵ is selected from the group consisting of alkyl, cycloalkyl, and haloalkyl.

28. The compound of any one of claims 1-6 and 13-27, wherein X¹⁰ is CH.

29. The compound of any one of claims 1-28, wherein R¹ is selected from the group consisting of H, alkyl, (C₃-C₈)cycloalkyl, alkoxyalkyl, hydroxyalkyl, heterocyclyl, heteroaryl, and haloalkyl, wherein the alkyl substituent is independently optionally substituted, at each occurrence, with a substituent selected from the group consisting of amine, halogen, cyano, (C₃-C₈)cycloalkyl, and heterocyclyl.

30. The compound of any one of claims 1-29, wherein R¹ is selected from the group consisting of H, alkyl, (C₃-C₈)cycloalkyl, alkoxyalkyl, heterocyclyl, and haloalkyl, wherein the alkyl substituent is independently optionally substituted, at each occurrence, with a substituent selected from the group consisting of amine, halogen, and (C₃-C₈)cycloalkyl.

31. The compound of any one of claims 1-30, wherein R¹ is selected from the group consisting of methyl and

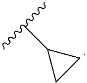 [SMILES: *C1CC1]

32. The compound of any one of claims 1-6 and 13-31, wherein R² is selected from the group consisting of alkyl and halogen.

33. The compound of any one of claims 1-6 and 13-32, wherein R² is selected from the group consisting of methyl and ethyl.

34. The compound of any one of claims 1-6 and 11-33, wherein R³ is selected from the group consisting of H, alkyl, alkoxy, and haloalkyl.

35. The compound of any one of claims 1--6 and 11-34, wherein R³ is selected from the group consisting of trifluoromethyl and H.

36. The compound of any one of claims 1-3 and 15-35, wherein R⁴ is H.

37. The compound of any one of claims 1-10 and 13-36, wherein R⁵ is selected from the group consisting of H, alkyl, alkoxy, amine, amide, haloalkyl, cycloalkyl, phosphine oxide, halogen, haloalkoxy, cyano, and cyanoalkyl.

38. The compound of any one of claims 1-10 and 13-37, wherein R⁵ is selected from the group consisting of H, trifluoromethyl, isopropyl, cyclopropyl, chloro,

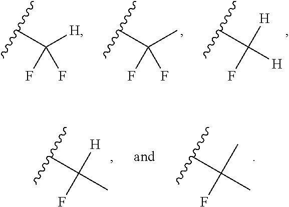

39. The compound of any one of claims 1-3 and 13-38, wherein L is optionally substituted C₁-C₆alkyl.

40. The compound of any one of claims 1-3 and 13-39, wherein L is selected from the group consisting of 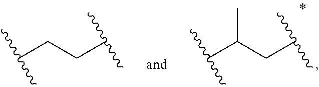
wherein E is attached to the carbon identified with *.

41. The compound of any one of claims 1-3 and 13-40, wherein L is selected from the group consisting of 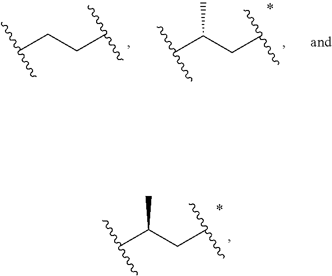
wherein E is attached to the carbon identified with *.

42. The compound of any one of claims 1-3 and 13-41, wherein E selected from the group consisting of H, methyl, and hydroxy.

43. A compound selected from the group consisting of: 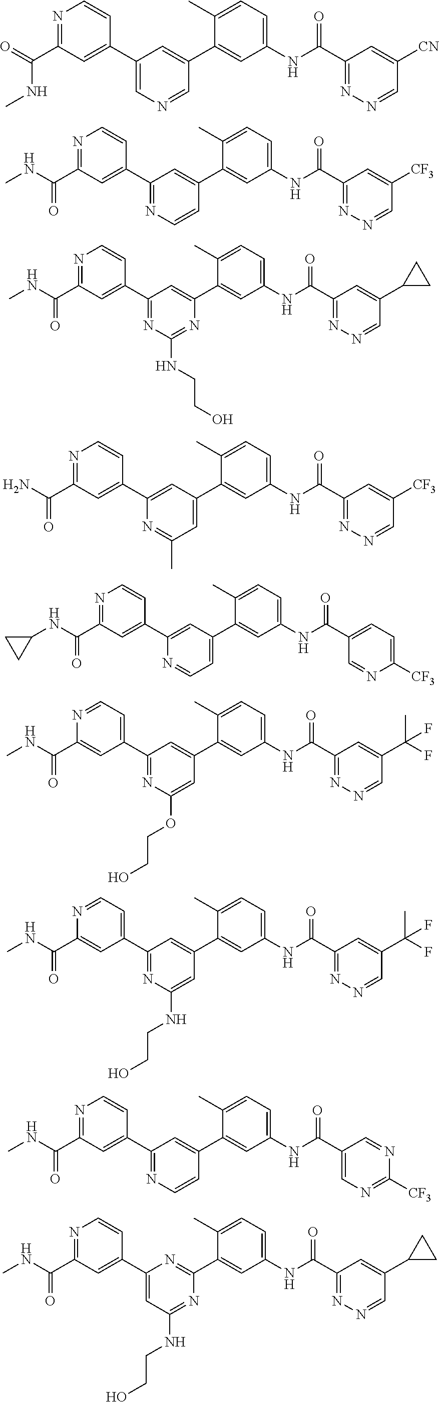 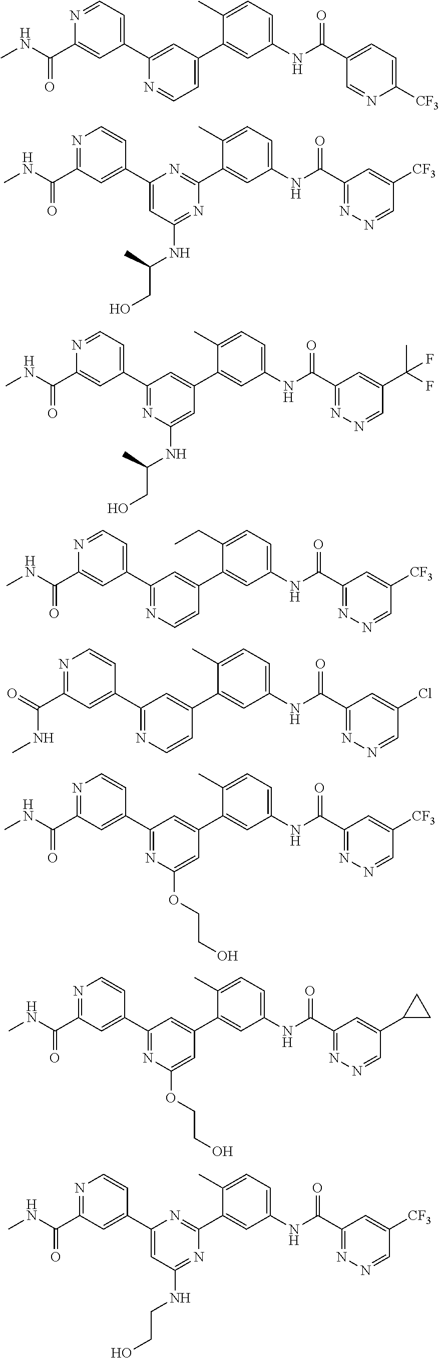 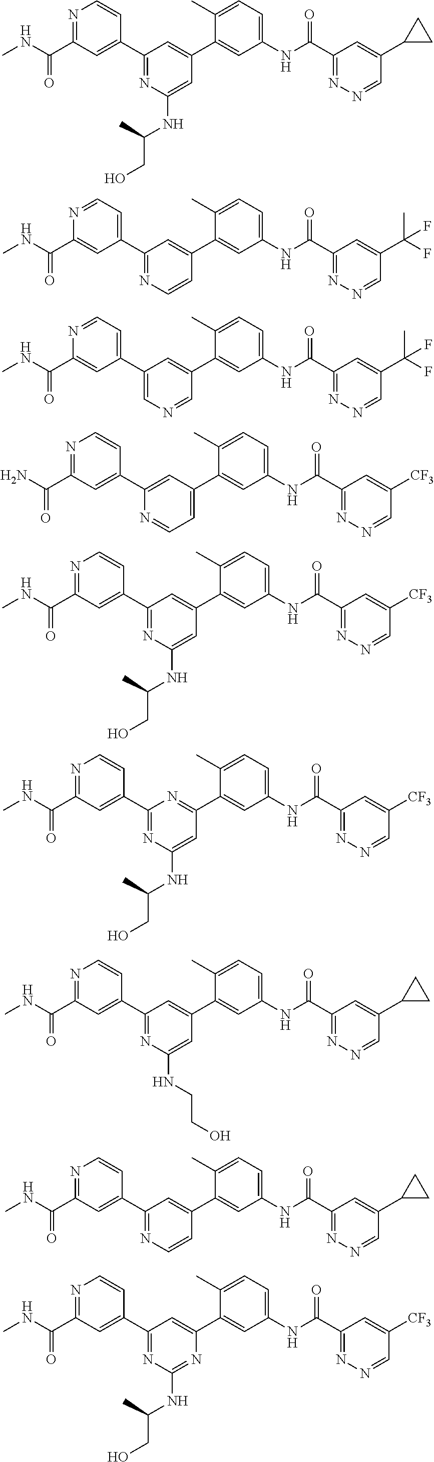 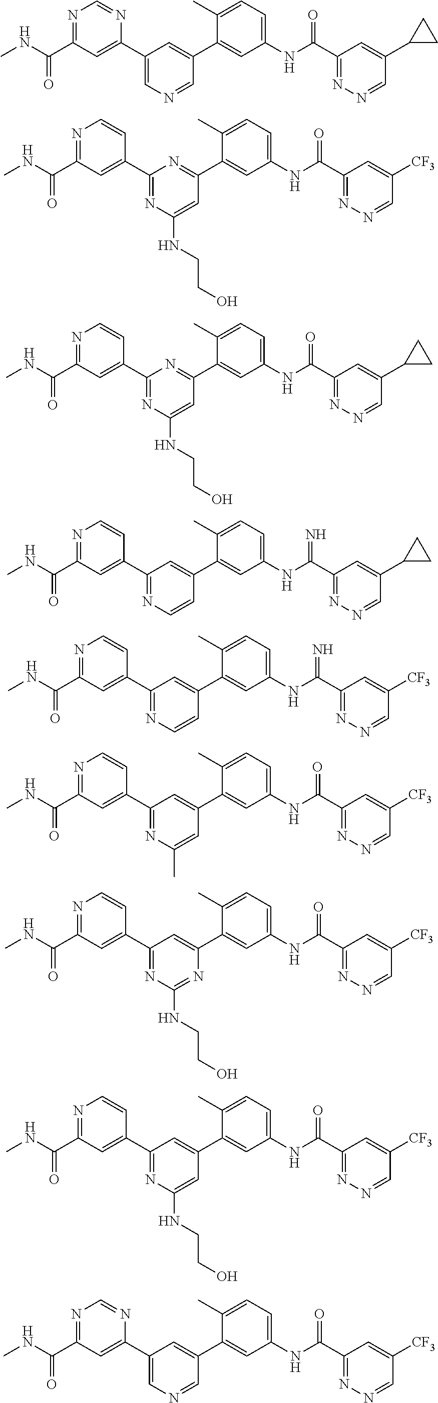 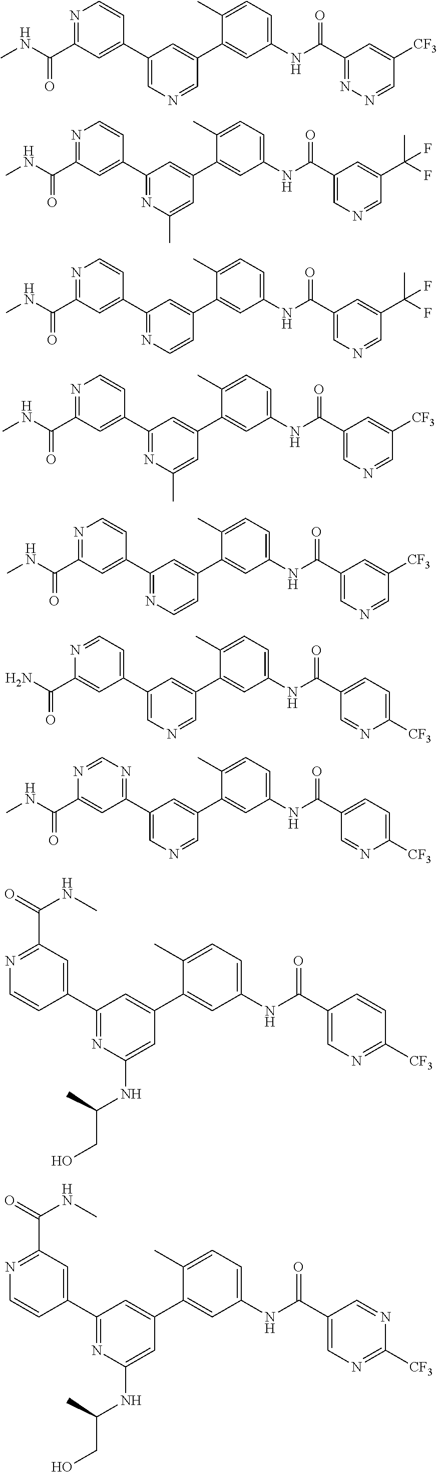 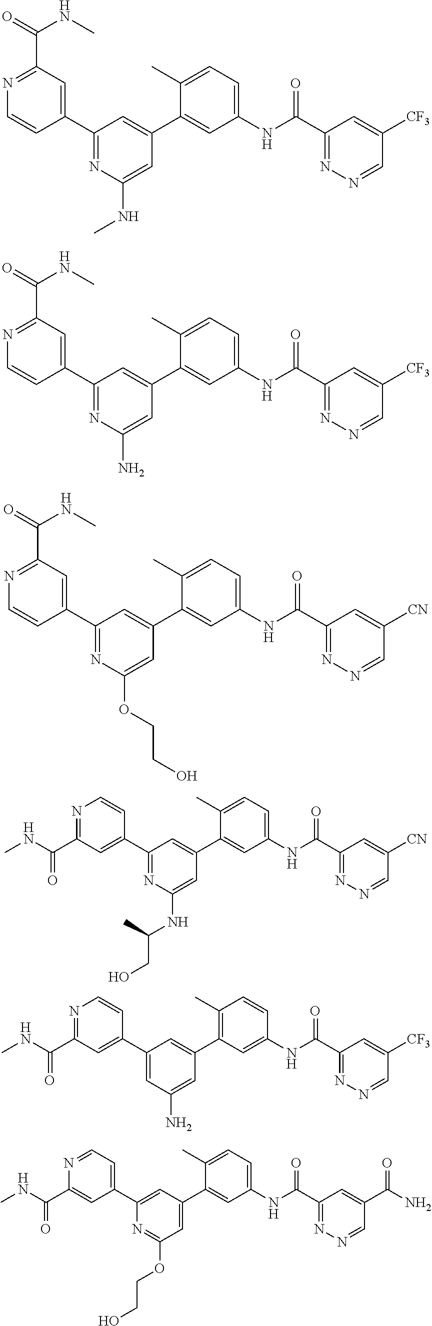 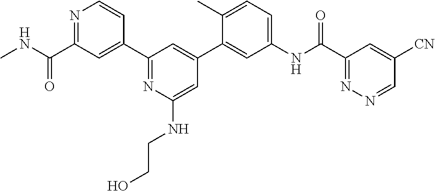 [SMILES: CNC(=O)c1cc(-c2cc(-c3cc(NC(=O)c4cc(C#N)cnn4)ccc3C)cc(NCCO)n2)ccn1]
and pharmaceutically acceptable salts, enantiomers, stereoisomers, and tautomers thereof.

44. A pharmaceutical composition comprising a compound according to any one of claims 1-43, or a pharmaceutically acceptable salt, enantiomer, stereoisomer, or tautomer thereof, and a pharmaceutically acceptable carrier or excipient.

45. A method of treating a cancer in a patient in need thereof, comprising administering to the patient a therapeutically effective amount of the compound of any one of claims 1-43, or a pharmaceutically acceptable salt, enantiomer, stereoisomer, or tautomer thereof, or of the pharmaceutical composition of claim 43.

46. The method of claim 45, wherein the cancer is selected from the group consisting of melanoma, multiple myeloma, thyroid cancer, ovarian cancer, colon cancer, pancreatic cancer, lung cancer, bladder cancer, gastrointestinal stromal tumors, solid tumors, brain cancers, gliomas, glioblastomas, astrocytomas, blood-borne cancers, acute myelogenous leukemia (AML), and other cancers caused by activation of the RAS->RAF->MEK->ERK signaling pathway.

47. The method of claim 45 or 46, wherein the cancer has a BRAF oncogenic mutation.

48. The method of any one of claims 45-47, wherein the cancer has a RAS oncogenic mutation.

49. The method of any one of claims 45-48, wherein the cancer has a NRAS oncogenic mutation.

50. The method of claim 49, wherein the NRAS oncogenic mutation is NRAS Q61R or NRAS Q61K mutation.

51. The method of any one of claims 45-50, wherein the cancer has a KRAS oncogenic mutation.

52. The method of claim 51, wherein the KRAS oncogenic mutation is KRAS G12D, KRAS G12V, KRAS G12C, KRAS G12R, or KRAS G13D.

53. The method of any one of claims 45-52, wherein the cancer has a NF1 oncogenic mutation.

54. A method of treating a disorder selected from the group consisting of melanoma, multiple myeloma, thyroid cancer, ovarian cancer, colon cancer, pancreatic cancer, lung cancer, bladder cancer, gastrointestinal stromal tumors, solid tumors, brain cancers, gliomas, glioblastomas, astrocytomas, blood-borne cancers, acute myelogenous leukemia (AML), and other cancers caused by activation of the RAS->RAF->MEK->ERK signaling pathway in a patient in need thereof, comprising administering to the patient a therapeutically effective amount of the compound of any one of claims 1-43, or a pharmaceutically acceptable salt, enantiomer, stereoisomer, or tautomer thereof, or of the pharmaceutical composition of claim 44.
